(19) United States
(12) Patent Application Publication
(10) Pub. No.: US 2023/0340486 A1
(43) Pub. Date: Oct. 26, 2023

(54) IN UTERO AND POSTNATAL GENE EDITING AND THERAPY FOR TREATMENT OF MONOGENIC DISEASES, INCLUDING MUCOPOLYSACCHARIDOSIS TYPE 1H AND OTHER DISORDERS

(71) Applicant: THE CHILDREN’S HOSPITAL OF PHILADELPHIA, Philadelphia, PA (US)

(72) Inventors: William H. Peranteau, Philadelphia, PA (US); Sourav Bose, Brookline, MA (US); Brandon White, Philadelphia, PA (US)

(73) Assignee: THE CHILDREN’S HOSPITAL OF PHILADELPHIA, Philadelphia, PA (US)

(21) Appl. No.: 18/006,975

(22) PCT Filed: Jul. 27, 2021
(86) PCT No.: PCT/US2021/071011

§ 371 Date: Jan. 26, 2023

Related U.S. Application Data
(60) Provisional application No. 63/056,954, filed on Jul. 27, 2020.

(51) Int. Cl. C12N 15/113; C12N 9/78; C12N 15/86

(52) U.S. Cl. CPC C12N 15/1137; C12N 9/78; C12N 15/86; C12Y 302/01076; C12N 2320/30; C12N 2750/14141

(57) ABSTRACT

A method for in utero and postnatal genome editing of a lysosomal storage disease gene, the method comprising administering to a subject an ABE or CBE complex, wherein the subject is an embryo, a fetus, a neonate, a child or an adult, ABE or CBE complex comprising CRISPR-mediated base editor and a guide RNA (gRNA), the gRNA targeting a mutation in a therapeutic gene; and introducing a modified codon in the therapeutic gene by base editing the therapeutic gene without inducing double strand DNA breaks, wherein the base editing is performed by the adenoviral vector, an adeno-associated viral vector, nucleoprotein complex or an mRNA in a lipid based nanoparticle.

### CROSS REFERENCE TO RELATED APPLICATION

This application claims priority to U.S. Provisional Application No. 63/056,954 filed Jul. 27, 2020, the entire disclosure being incorporated herein by reference as though set forth in full.

### GRANT STATEMENT

This invention was made with government support under grant number DP2HL152427 awarded by the National Institutes of Health. The government has certain rights in the invention.

### INCORPORATION-BY-REFERENCE OF MATERIAL SUBMITTED IN ELECTRONIC FORM

Incorporated herein by reference in its entirety is the Sequence Listing submitted via EFS-Web as a text file named SEQLIST.txt, created on Jul. 27, 2021 and having a size of 19,295 bytes.

### FIELD OF THE INVENTION

This invention relates to the fields of genetic disease and gene editing technology. More specifically, the invention provides compositions and methods for correcting gene sequences in an embryo, a fetus, a neonate, a child or an adult, thereby ameliorating symptoms of monogenic disorders, including mucopolysaccharidosis type I storage disease and other lysosomal storage diseases (LSD), before or after birth.

### BACKGROUND OF THE INVENTION

Several publications and patent documents are cited throughout the specification in order to describe the state of the art to which this invention pertains. Each of these citations is incorporated by reference herein as though set forth in full.

A monogenic disease or a monogenic disorder is a condition determined by the interaction of a pair of alleles of single genes. This is in contrast to a polygenic condition wherein several genes (polygene) are involved. In humans, the monogenic disease is less frequent than the polygenic disease. It is also less complicated than the latter and may follow a pattern based on Mendelian inheritance. Since monogenic disease involves a single pair of genes the dysfunctional or mutated gene may be identified more easily than polygenic disease. Such diseases are particularly amenable to ABE and CBE approaches.

LSDs are often mongenic and can affect multiple organs, have limited treatments, and have pathology that often begins before birth^{1,2}. In MPS-IH for example, IDUA gene mutations cause α-L-iduronidase (IDUA) deficiency and lysosomal accumulation of glycosaminoglycans (GAGs). One of the most common mutations (G→A; tryptophan→stop; W402X) results in undetectable IDUA in the homozygous state and has a strong genotype-phenotype correlation^{3,4}. Children present by 6 months of age with hepatosplenomegaly, abdominal wall hernias, musculoskeletal abnormalities, retinal and neurocognitive degeneration, and cardiac disease^{5.} Current treatments include costly, lifelong, immunogenic enzyme replacement therapy and hematopoietic stem cell transplantation (HSCT), which is limited by donor availability, graft failure, graft-versus-host disease, and complications of myeloablation/immunosuppression^{5.} Neither treatment resolves musculoskeletal and cardiac pathologies^{5,6}, which significantly contribute to MPS-IH clinical grade⁷. Without treatment, patients die of cardiorespiratory complications by 5-10 years-of-age^{5,6,} ⁸⁻¹⁰

Gene therapy and editing may treat MPS-IH by augmenting IDUA expression in diseased organs or by enhancing liver IDUA secretion for systemic uptake. Postnatal systemic gene therapy and editing studies in the Idua^{-l-} mouse model are encouraging. Intravascular AAV and retroviral delivery of the Idua transgene mitigated the skeletal, metabolic, neurologic, cardiac, ear, and eye disease phenotypes¹¹_{′}¹², but this approach is limited by potential loss of an episomal transgene and insertional mutagenesis. Similarly, AAV-mediated zinc-finger nuclease editing to express Idua in the hepatocyte Albumin locus of adult mice caused enhanced IDUA secretion, decreased tissue GAGs, and improved neurobehaviour¹³. Finally, neonatal hydrodynamic intravascular liposomal delivery of CRISPR-Cas9 targeting the hepatocyte Rosa26 locus for homology-directed repair (HDR) with Idua integration partially improved GAGs, serum IDUA, and skeletal and cardiac disease¹⁴. Although encouraging, postnatal CRISPR-HDR is inefficient and requires double-stranded DNA breaks (DSBs) that are associated with unwanted mutagenesis, large deletions, and complex rearrangements at on- and off-target sites¹⁵⁻¹⁷

Base editing is a CRISPR editing approach that can convert adenine to guanine in a site-specific fashion without the need for DSBs or HDR templates. The adenine base editor (ABE) comprises a catalytically-impaired Streptococcus pyogenes Cas9 (SpCas9) and a modified tRNA adenine deaminase¹⁸_{′}¹⁹. The SpCas9 guide RNA (gRNA) tethers the ABE to the target site, and the adenine deaminase converts a nearby adenine to hypoxanthine and, ultimately, guanine. Unlike HDR, adenine base editing does not require cells to be proliferating to efficiently introduce mutations and has infrequent unwanted on- and off-target mutagenesis²⁰⁻²³, offering a potentially safer, more efficient correction of the MPS-IH G→A mutation.

The presentation of MPS-IH symptoms by 6 months of age argues for the prenatal onset of pathology². Mid-gestation MPS-IH fetuses have demonstrated metabolic and histopathologic evidence of disease in multiple organs including the liver, heart, and brain²⁴⁻²⁶. Furthermore, prenatal cardiac dysfunction leads to myocardial hypertrophy and early postnatal death in MPS-IH²⁷. Prenatal onset of pathology, early progressive postnatal morbidity, and feasibility of prenatal diagnosis suggest a potential benefit to MPS-IH prenatal therapy. It is an object of the invention to provide such a therapy.

### SUMMARY OF THE INVENTION

In accordance with the present invention, an adenine base editor (ABE) complex for programming conversion of adenine to guanine in a patient in need thereof is provided wherein the patient has a target DNA molecule harboring a mutation associated with a monogenic disease. In certain embodiments, the ABE complex comprises a modified TadA enzyme, a catalytically impaired Cas 9 protein and a guide RNA (sgRNA) which directs said ABE complex to the mutated target DNA molecule, which upon contact converts adenine in said mutation to inosine, thereby catalyzing an A-T to G-C transition following DNA replication. Notably, no double strand breaks occur in the targeted DNA and homologous DNA recombination is not required. The ABE portion of the complex can include without limitation ABEmax, ABE6.3, ABE6.4, ABE7.8, ABE7.9, ABE7.10, ABE7.10-m, ABE7.10-d, ABE8.8-m, ABE8.8-d, ABE8.13-m, ABE8.13-d, ABE8.17-m, ABE8.17-d, ABE8.20-m and ABE8.20-d. The catalytically impaired Cas9 protein can be, but is not limited to NRRH, NRTH, NRCH, xCas9, SpCas9-NG, SpCas9, SpG, SpRY, SauriCas9, SaCas9, Nme2Cas9, VRER-SpCas9, and VQR-SpCas9. While treatment of Hurler syndrome is exemplified herein, these compositions and methods disclosed herein can be expanded to treat any disorder where a single base change corrects the mutation and restores the wild-type phenotype.

Using Hurler syndrome as an example, suitable protospacer and PAM sequences can be selected from SEQ ID NOS: 1-8 shown below.
i) spCas9.ABEmax and GCTCTAGGCCGAAGTGTCGC AGG;
ii) spCas9.ABEmax and TAGGCCGAAGTGTCGCAGGC and CGG;
iii) Nme2Cas9.ABEmax and GAGCAGCTCTAGGCCGAAGTGTCG and CAGGCC;
iv) Nme2Cas9.ABEmax and CTCTAGGCCGAAGTGTCGCAGGCC and GGGACC;
v) SpG.ABEmax CTCTAGGCCGAAGTGTCGCA and GGCC;
vi) SpRY.ABEmax CTCTAGGCCGAAGTGTCGCA and GGCC;
vii) SauriCas9.ABEmax AGCTCTAGGCCGAACTCTCG and CAGG; and
viii) NRCH.ABEmax GCAGCTCTAGGCCGAAGTGT and CGCA.

The aforementioned ABE complex is useful for correcting a target DNA sequence which comprises a W402X mutation in the Idua gene and said G→A conversion restores Idua activity.

In a particularly preferred embodiment, the ABE portion of the complex is ABE7.10 and the guide strand comprises a protospacer and PAM sequence of 5′GCTCTAGGCCGAAGTGTCGC AGG3′ (SEQ ID NO: 1), which is effective to restore Idua activity and ameliorate symptoms of Hurler syndrome.

Several different methods of administration may be employed to deliver the gene editing complexes to the subject, depending on the nature of the disorder and the tissue to be treated. These include delivery of plasmid and viral vectors, as well as nucleoprotein complexes or mRNA encoding the editing complex in particles or liposomes or in lipid based nanoparticles. In certain embodiments for example when AAV9 is used, the binding complex is delivered in two vectors given size constraints for inserts into AAV9. Upon entry into the cells, the editing complex reforms to edit the site targeted by the guide strand. Nanoparticle (NP) technology including, but not limited to, lipid nanoparticles, are used to deliver the base editor and guide RNA targeting the therapeutic gene including the MPS-IH mutation. While use of an ABE is exemplified herein, a cytosine base editor (CBE) can also be employed. CBEs mediate a C to T change (or a G to A change on the opposite strand) in the human genome when appropriate. This approach involves encapsulating the adenine base editor (ABE) or cytosine base editor (CBE) mRNA in one delivery vehicle, (e.g., a viral vector, plasmid, nanoparticle (NP), liposome, and lipid nanoparticle) and encapsulating the guide RNA in a second delivery vehicle such as one or more of those listed above. Both delivery vehicles are subsequently delivered to the recipient, either fetal or postnatal recipient, via a route most suitable for the particular disorder to be treated. When Hurler’s syndrome is to be treated, intravascular delivery or intra-cranial delivery routes are preferred. As an alternative, both the base editor mRNA and the guide RNA are encapsulated in a single delivery vehicle which is then delivered intravascularly to the prenatal or postnatal recipient. In certain approaches, the ABE or CBE ribonucleoprotein (RNP) is encapsulated in one NP and the guide RNA is encapsulated in a second NP. Both NPs are subsequently delivered to the recipient, either fetal or postnatal recipient, via a route most suitable for the particular disorder to be treated including intravascular and intracranial delivery for Hurler’s syndrome. In certain embodiments, the nanoparticle is complexed with a lipid, forming a lipid nanoparticle. In another embodiment, the ABE or CBE RNP and the guide RNA are encapsulated in a single NP or lipid nanoparticle, which is then delivered to the prenatal or postnatal recipient via a route most suitable for treatment of the particular disorder to be treated including intravascular and intracranial delivery for Hurler’s syndrome.

Also provided is a method for genome editing of a mutated gene sequence associated with a monogenic disorder, including without limitation, lysosomal storage diseases (LSD) in a patient in need thereof. An exemplary method comprises administering to the patient an ABE complex as described herein which introduces a modified codon into the targeted gene sequence and corrects the LSD mutation in this exemplary embodiment, wherein the base editing does not induce double strand breaks in the target nucleic acid and said correction of the mutation ameliorates symptoms of said lysosomal storage disease. The patient can be a fetus, a neonate, a child or an adult. In some embodiments, the targeted gene sequence is edited in an embryo. In certain embodiments, the base editing occurs prior to Hurler syndrome onset. In other embodiments, the base editing decreases risk of developing a disease.

The method of the invention can entail use of first and second AAV9 vectors as delivery vehicles wherein amplified N- and C-termini of the ABEmax are ligated at SpCas9 Glu573 and Cys574 to codon optimized N- and C-termini of the Npu intein, respectively. Codons for Leu564 and Lys565 in the Npu intein being altered to an AflII site (CTT|AAG) to form plasmids which comprise a CBh promoter and WPRE3-bGH polyadenylation signal sequences which are inserted between AAV ITRs in said first and second vectors.

Also provided is a cytosine base editor (CBE) complex for programming conversion of cytosine into a thymine in a patient in need thereof said patient have a target DNA molecule harboring a mutation associated with a lysosomal storage disease comprising, a cytosine deaminase domain, a catalytically impaired Cas 9 protein and a single guide RNA (sgRNA) which directs said CBE complex to said mutated target DNA molecule, which upon contact converts cytosine in said mutation to inosine, thereby catalyzing an C→T transition following DNA replication. In certain embodiments, the catalytically impaired Cas9 protein in the CBE complex is selected from NRRH, NRTH, NRCH, xCas9, SpCas9-NG, SpCas9, SpG, SpRY, SauriCas9, SaCas9, Nme2Cas9, VRER-SpCas9, and VQR-SpCas9. In some embodiments, the CBE portion of the complex is selected from APOBEC1, E63A, CDA, AID, A3A, A3B, A3G, YE1, YE2, YEE, EE, R33A, eA3A, FERNY, BE3, BE4, and BE4max.

Another aspect of the invention includes a method for treating a lysosomal storage disease in a fetal, neonate, child or adult subject, the method comprising identifying in vitro a target codon for base editing; providing an ABE or CBE complex, and administering said complex to the subject; thereby introducing a modified codon in the identified mutated gene sequence and ameliorating symptoms of said lysosomal storage disease in said subject.

Monogenic disorders that can be treated using the compositions and methods of the invention include any that would benefit from an ABE or CBE gene editing event.

The invention also provides kits comprising components and reagents for practicing the methods disclosed above.

### BRIEF DESCRIPTION OF THE DRAWINGS

FIG. 1: Flow cytometry plots used for sorting CD45⁻LGR5⁺ liver cells, CD45⁻CD31⁻CD90.2⁺ cardiac fibroblasts, and CD45⁻CD31⁺CD90.2⁻ cardiac endothelial cells.

FIGS. 2A-2Z: In utero CRISPR-mediated nonhomologous end joining (NHEJ) following split-intein AAV9 delivery in the R26m^{TmG/+} mouse model. E15.5 R26m^{TmG/+}fetuses were injected with split-intein AAV9s containing the SpCas9 transgene and gRNA targeting the loxP sites flanking the mT cassette (AAV. SpCas9.mTmG). Successful excision of the mT cassette and repair via NHEJ results in expression of green fluorescence. FIGS. 2A-2P The hearts and livers of prenatally injected mice and uninjected R26m^{TmG/+} controls were analysed at 1 week-of-age by stereomicroscopy (experimental heart, a-d; control heart, e-h; experimental liver, i-l; control liver, m-p) for GFP expression. (A-P) Scale bar = 1 mm. FIGS. 2Q-2R Hearts and livers of day-of-life 1 and 1 week old R26m^{TmG/+} mice prenatally injected with AAV.SpCas9.mTmG were assessed by flow cytometry for GFP expression. FIGS. 2S-2Z Whole mount IHC of heart with staining for troponin (yellow, S-V) and the liver with staining for LGR5 (white, W-Z) in 1 week old R26m^{TmG/+} mice prenatally injected with AAV.SpCas9.mTmG (T,V,X,Z) and uninjected R26m^{TmG/+} mice (S,U,W,Y). (S-V) Scale bar = 50 µm. Red-filled arrowheads identify cardiomyocytes. (W-Z) Scale bar = 25 µm. White arrowheads identify LGR5+ cells. EGFP, green; TdTomato, red; cTnT, cardiac troponin, yellow; LGR5, leucine-rich repeat-containing G-protein receptor 5, white.

FIGS. 3A-3M: In utero base editing in the Idua-W392X MPS-IH mouse model. FIG. 3A Sanger sequencing of liver genomic DNA for E15.5 AAV.ABE.Idua injected (bottom panel) and uninjected (top panel) Idua-W392X fetuses at 1 month of age. PAM and protospacer in red and blue respectively. On-target A→G edit noted. SEQ ID NO: 9 is shown. FIG. 3B Scheme for long-term genetic, biochemical, and phenotypic studies. FIG. 3C NGS, genomic DNA from indicated organs at 6 months of age. FIG. 3D NGS, genomic DNA from whole liver and sorted CD45⁻LGR5⁺ liver cells at 6 months of age. FIG. 3E NGS, genomic DNA from whole heart and isolated cardiac myocytes, endothelial cells, and fibroblasts at 6 months of age. FIG. 3F NGS of the Idua on-target site and the top 10 predicted off-target sites in liver genomic DNA at 6 months of age from 2 AAV.ABE.Idua recipients and 1 control. SEQ ID NOS: 10-20 are shown in descending order. FIG. 3G Frequencies of base-edited and indel-bearing alleles assessed at 6 months of age via NGS of liver genomic DNA of in utero AAV.ABE.Idua recipients (N=10). Underlined bases indicate the target codon. FIGS. 3H-3M Representative IHC of brain, heart, and liver of uninjected (H-J) and prenatally AAV.ABE.Idua (K-M) injected 6-month-old Idua-W392X mice with antibody specific for the C-terminus of IDUA (red). Scale bar = 25 mm. (C-F) Control = uninjected 6-month-old Idua-W392X mice. (C-E) Experimental N (red marks) = 10 except for cardiomyocyte, endothelial, and cardiac fibroblast in which experimental N = 6; Control N = 7. AAV.ABE, represents mice injected with AAV.ABE.Idua.

FIGS. 4A-4F: Durable improvement in biochemical parameters in Idua-W392X mice following in utero base editing. FIG. 4A Urine GAGs were measured monthly in B6 mice (N=14), Idua-W392X mice prenatally injected with AAV.ABE.Idua (N=10), and uninjected Idua-W392X mice (N=14). FIGS. 4B-4C Tissue GAGs were measured at 6 months of age in the heart and liver (B) and other indicated organs (C) in B6 mice (N=14), prenatally injected mice (N=10), and uninjected mice (N=14, except eye, N=13). FIG. 4D Serum IDUA activity was measured at 6 months of age in B6 mice (N=10), prenatally injected mice (N=10), and uninjected mice (N=10). FIGS. 4E-4F Tissue IDUA activity was measured at 6 months of age in the heart and liver (E) and other indicated organs (F) in B6 (N=14), prenatally injected mice (N=10), and uninjected mice (N=14). ^, p<0.0001; #, p<0.001; *, p<0.05. Wilcoxon test for multiple comparisons used to assess urine GAG months 1-3, liver GAG, 6-month serum IDUA activity, and heart and liver IDUA activity. All remaining statistical analyses used Student’s t-test with α=0.05. GAG, glycosaminoglycans; IDUA, α-L-iduronidase.

FIGS. 5A-5S: In utero base editing diminishes aberrant tissue GAG deposition in brain, heart, and liver. FIGS. 5A-5S IHC with Alcian blue staining, which highlights accumulated GAGs, was performed in the brain, heart, and liver (three primary MPS-IH affected organs) in B6 mice (N=14), Idua-W392X mice prenatally injected with AAV.ABE.Idua (N=10), and uninjected Idua-W392X mice (N=14). Representative sections: 40x brain sections featuring axonal tracts and periaxonal cells near cerebellar peduncles (A, G, M); 63x brain sections featuring axonal tracts and periaxonal cells near cerebellar peduncles (B, H, N); 40x left ventricles (C, I, O); 63x left ventricles (D, J, P); 40x liver (E, K, Q); and 63x liver (F, L, R). (S) Alcian blue staining was quantified and normalized to number of visualized nuclei per hpf. Arrowheads denote Alcian blue staining of GAGs in vacuolated cells in the brain, the interstitial space in the heart, and in hepatocytes. (A, G, M, C, I, O, E, K, Q) Scale bar = 50um. (B, H, N, D, J, P, F, L, R) Scale bar = 10 µm. GAG, glycosaminoglycans. AAV.ABE indicates AAV.ABE.Idua.

FIGS. 6A-6T: In utero base editing improves cardiac function and survival in Idua-W392X mice. FIGS. 6A-6D Echocardiographic parameters were measured in 4-month-old B6 mice (N=10), Idua-W392X mice prenatally injected with AAV.ABE.Idua (N=10), and uninjected Idua-W392X mice (N=10). Mice that died prior to 6 months of age are denoted with a green X. FIGS. 6E-6H Echocardiographic parameters were measured in 6-month-old B6 mice (N=10), prenatally injected mice (N=10), and surviving uninjected mice (N=6). FIGS. 6I-6L Ascending aorta diameter and representative ultrasounds from 4-month-old B6 mice (N=10), prenatally injected mice (N=10), and uninjected mice (N=10). Mice that died prior to 6 months of age are denoted with a green X. Scale bar = 1 mm. FIGS. 6M-6P Ascending aorta diameter and representative ascending aorta ultrasounds from 6-month-old B6 mice (N=10), prenatally injected mice (N=10), and surviving uninjected mice (N=6). Scale bar = 1 mm. FIGS. 6Q-6S Representative Masson’s trichrome stain highlighting collagen deposition in aortic outlets including the aortic valve (*) and ascending aorta (black and white arrow) in 6-month-old B6 mice, prenatally injected mice, and uninjected mice. Red stains muscle, pink stains cytoplasm, black stains nuclei, and blue stains collagen. Images constructed with 40x tile stitch. Scale bar = 200 µm. FIG. 6T Six-month survival analysis comparing B6 mice (N=10), prenatally injected mice (N=10), and uninjected mice (N=10). Wilcoxon test for multiple comparisons used to assess ascending aorta diameter; Mantel-Cox method used to assess survival; Student’s t-test used for all other comparisons; α=0.05. LV left ventricle. Ao, aorta. AAV.ABE indicates AAV.ABE.Idua.

FIGS. 7A-7O: In utero base editing improves the musculoskeletal and neurobehavioural phenotype in Idua-W392X mice. FIGS. 7A-7C CT face parameters were measured in 6-month-old B6 mice (N=10), 6-month-old Idua-W392X mice prenatally injected with AAV.ABE.Idua (N=10), and surviving uninjected Idua-W392X mice (N=6). FIGS. 7D-7F 3D CT reconstructions of representative skulls from 6-month-old B6 mice, prenatally injected mice, and surviving uninjected mice with snout angle and zygomatic arch measurements indicated. Scale bar = 5 mm. FIGS. 7G-7I CT femur parameters were measured in 6-month-old B6 mice (N=10), prenatally injected mice (N=10), and surviving uninjected mice (N=6). FIGS. 7J-7L 3D CT reconstructions of representative femurs from 6-month-old B6 mice, prenatally injected mice, and uninjected mice demonstrating cortical thickness measurements and surface contour. Scale bar = 1 mm. FIG. 7M Grip strength was measured in 6-month-old B6 mice (N=10), prenatally injected mice (N=10), and surviving uninjected mice (N=6). FIGS. 7N-7O Open field test measurements of habituation between trials of testing in 6-month-old B6 mice (N=10), prenatally injected mice (N=10), and surviving uninjected mice (N=6). Wilcoxon test for multiple comparisons was used to assess femur cortical area and thickness; Student’s t-test used for all other comparisons; α=0.05. CT, computed tomography scan. AAV.ABE indicates AAV.ABE.Idua.

FIGS. 8A-8I: In utero base editing ameliorates the facial and skeletal abnormalities in Idua-W392X mice. FIGS. 8A-8I Representative photographs of 6-month-old female B6 mice (A, D, G), Idua-W392X mice prenatally treated with AAV.ABE.Idua (B, E, H), and untreated Idua-W392X mice (C, F, I). Scale bar = 1 cm.

FIGS. 9A-9I: Base editing in adult Idua-W392X mice partially corrects biochemical and cardiac abnormalities. 10-week-old Idua-W392X mice were intravascularly injected with AAV.ABE.Idua. FIG. 9A A liver biopsy was performed at 4 months of age, and genomic DNA was assessed by NGS for on-target Idua editing (N=5). W392X = uninjected controls (N=7). FIG. 9B Prior to adult injection, urine was collected for baseline urine GAG levels (2.5 month values) and urine GAGs were subsequently measured in B6 mice (N=14), adult Idua-W392X injected mice (N=5), prenatally injected Idua-W392X mice (N=10), and uninjected Idua-W392X mice (N=14) at 3, 4, and 5 months of age. FIGS. 9C-9D Liver GAGs (C) and IDUA activity (D) were measured from the liver biopsy specimen in adult Idua-W392X injected mice and compared to levels from B6 mice (N=14), prenatally injected Idua-W392X mice (N=10), and uninjected Idua-W392X mice (N=14). FIGS. 9E-9I Adult injected Idua-W392X mice (N=5) underwent echocardiography at 4 months of age, and the ascending aorta diameter (E), aortic valve diameter (F), left ventricle diameter (G), LV ejection fraction (H), and LV fractional shortening (I) were compared to those in 4-month-old B6 mice (N=10), prenatally injected Idua-W392X mice (N=10), and uninjected Idua-W392Xmice (N=10). ^, p<0.0001; #, p<0.001; *, p<0.05. Wilcoxon test for multiple comparisons used to assess 3-month urine GAGs, liver IDUA activity, and anti-SpCas9 antibody. All remaining statistical analyses used Student’s t-test with α=0.05. GAG, glycosaminoglycans; IDUA, a-L-iduronidase. AAV.ABE indicates AAV.ABE.Idua.

FIG. 10: Base editing in adult Idua-W392X mice is associated with anti-SpCas9 specific antibodies. 10-week-old Idua-W392X mice were intravascularly injected with AAV.ABE.Idua. Sera from Idua-W392X mice prenatally (N=10) or postnatally (N=5) treated with AAV.ABE.Idua were harvested at one month post-injection and assessed for anti-SpCas9 antibodies. W392X = uninjected control serum (N=10). ^, p<0.0001; #, p<0.001; *, p<0.05. Wilcoxon test for multiple comparisons used to assess 3-month urine GAGs, liver IDUA activity, and anti-SpCas9 antibody. All remaining statistical analyses used Student’s t-test with α=0.05. GAG, glycosaminoglycans; IDUA, α-L-iduronidase. AAV.ABE indicates AAV.ABE.Idua.

FIG. 11: Schematic diagram of canine study.

FIG. 12: A blot showing that restriction digest provides an accurate approach for same day assessment of effective editing.

FIG. 13: Scheme for genetic, biochemical, and phenotypic studies in MPS I-H primary patient derived fibroblasts and 10 week-old Idua-W392X mice.

FIGS. 14A-14F: ABE.IDUA efficiently and specifically corrects the genomic disease pattern in MPS-IH primary patient derived fibroblasts. FIG. 14A Primary fibroblasts were transfected with ABE7.10 mRNA and gRNA targeting the W402X mutation. On Sanger sequencing a precise base correction was noted on target in ABE.IDUA W402X treated fibroblasts compared to W402X untreated fibroblasts. (SEQ ID NO: 97 is shown) FIG. 14B On-target DNA editing in ABE.IDUA W402X treated fibroblasts was ~60% with ~0% insertions and deletions. FIG. 14C The most common substitutions noted within the ABE editing window in treated ABE.IDUA W402X fibroblasts. FIG. 14D On-target mRNA correction associated with DNA editing was ~45% in ABE.IDUA W402X treated fibroblasts. FIG. 14E Relative gene expression for a clinical biomarker panel for MPS-IH in ABE.IDUA W402X treated fibroblasts compared to untreated W402X fibroblasts demonstrates a restoration of gene expression in multiple cellular pathways. FIG. 14F Gene ontology profile of the top 100 genes expressed in ABE.IDUA W402X treated fibroblasts compared to untreated W402X fibroblasts.

FIGS. 15A-15D: Genomic correction results in restoration of biochemical function and immune state. FIGS. 15A-15B Media and cell IDUA activity over 4 weeks post transfection (ng/h/mL media or log ng/h/mg protein respectively); dotted lines demarcate zone of transition from hurler (severe) to scheie (mild) phenotype in humans (Hopwood, J. J., & Muller, V. (1979). Biochemical discrimination of Hurler and Scheie syndromes. Clinical Science, 57(3), 265-272). FIG. 15C Intracellular and membrane gag levels measured at 4 weeks after transfection. FIG. 15D Expression of inflammatory genes measured by quantitative reverse transcriptase PCR; exact p values in supplementary table; normalized to gapdh. Blue squares represent IDUA activity in ABE.IDUA W402X treated fibroblasts, black circles represent IDUA activity in normal human dermal (NHD) fibroblasts, and red triangles represent IDUA activity in W402X fibroblasts. Statistical comparisons with Tukey Kramer HSD with α=0.05.

FIGS. 16A-16B: ABE.IDUA diminishes the lysosomal burden in MPS-1H. FIG. 16A LAMP-1 expression as measured by immunoblot in treated ABE.IDUA W402X, NHD, and W402X fibroblasts 4 weeks after transfection. FIG. 16B Lysosomal staining was evaluated by fluorescence activated cell sorting — treated fibroblasts had a statistically significantly reduced signal suggesting fewer and smaller lysosomes. FIGS. 16C-16E Demonstration of reduced lysosomal burden in ABE.IDUA W402X treated fibroblasts. Immunocytochemistry using lysotracker DND-26 staining. Statistical comparisons with Tukey Kramer HSD with α=0.05.

FIGS. 17A- 17B: ABE.IDUA results in no appreciable DNA off-targeting as measured by multiple targeted and whole genome assessments. FIG. 17A Karyotype with 45 sites of potential off-target activity. FIG. 17B Base edits and indels at 45 off-target loci were not detected in ABE.IDUA W402X treated fibroblasts.

FIGS. 18A- 18B: Unbiased Induce-Seq and Dig-Seq assessment of DNA off-targeting in human W402X fibroblasts with the human MPSIH IDUA gRNA. W402X primary human fibroblasts were transfected with Cas9-gRNA ribonucleoprotein targeting the W402X mutation. FIG. 18A Guide RNA directed double stranded breaks and consequent insertions and deletions in treated W402X fibroblasts subject to whole genome sequencing for identification of off-targets for Dig-Seq and Induce-Seq. FIG. 18B Mismatch count at loci identified to have Cas9-gRNA ribonucleoprotein activity as measured by Induce-Seq. (SEQ ID NO: 1 is shown).

FIGS. 19A – 19D: ABE.IDUA results in low level transcriptome RNA off-target activities with minimal predicted deleterious effects. FIG. 19A Transcriptome wide A to I variation in whole transcriptome RNA sequencing of NHD, W402X, nickase Cas9 treated, and ABE.IDUA treated W402X fibroblasts. FIG. 19B Classification of unique A to I editing sites in transcriptome wide sequencing. FIG. 19C Nature of variation of A to I RNA editing sites in coding regions. FIG. 19d Predicted consequence of A to I editing in nonsynonymous coding regions based on SIFT scores.

FIGS. 20A – 20E: Three Lipid nanoparticles can effectively package and deliver functional ABE in vitro and in vivo. FIG. 20A Three LNP amine cores were evaluated for ability to target human HUH7 liver cells. LNP C12-200, A3, and B3 encapsulating eGFP mRNA were delivered to 70% confluent 24-well plated cells at a dose of 400 ng. At 24 hours after transfection, GFP was quantified by fluorescence activated cell sorting. FIG. 20B Optimization of LNP A3-mediated editing was assessed by modulating ABE7.10 mRNA to gRNA ratio. A total 400 ng of mRNA/gRNA was applied to 150000 human W402X fibroblasts. Editing was assessed at 24 h hours by Sanger Sequencing. FIG. 20C Human W402X fibroblasts were treated with 1 ug LNP A3 encapsulating ABE7.10 mRNA and gRNA targeting the W402X mutation at a 1:1 ratio and assessed at 60 hours after transfection using Sanger Sequencing. (SEQ ID NO: 21 is shown) FIG. 20D Four 10 week old W392X mice were intravascularly injected with LNP A3 encapsulating ABE7.10 and gRNA targeting the W392X mutation every other day for 3 doses of 1.5 mg/kg each. Editing was assessed 1 week after the final dose in brains, hearts, and livers of treated mice and measured by NGS. FIG. 20E Brains, hearts, and livers of LNPA3 treated mice were assessed for IDUA activity 1 week after the final dose. Dotted lines mark the transition zone from severe to mild disease based on enzyme activity.

### DETAILED DESCRIPTION OF THE INVENTION

The developing fetus has many properties that make it ideal for in vivo base editing. Small fetal size allows delivery of high-dose gene-editing technology per weight; somatic and progenitor cells of multiple organs are accessible for efficient viral transduction²⁸⁻³⁰; the nascent and permissive blood-brain barrier (BBB) facilitates systemic access to the central nervous system (CNS)^{31′32}; and the immune system is tolerant. Multiple studies have demonstrated the lack of an immune response to the viral vector and transgene product, including SpCas9, following in utero gene therapy/editing in contrast to postnatal treatment³³⁻³⁹. Furthermore, Cas9-specific immunity has been demonstrated in humans after birth⁴⁰ and has eliminated edited hepatocytes following AAV-mediated Cas9 delivery in mouse models⁴¹. Finally, in utero base editing offers the potential to treat MPS-IH prior to the onset of irreversible pathology.

We previously demonstrated that adenovirus-mediated in utero base editing efficiently introduces a nonsense mutation in the Hpd gene in hepatocytes and rescues the lethal phenotype in the hereditary tyrosinemia type 1 mouse model³⁷. There is a tremendous survival advantage of corrected hepatocytes in that model. While adenovirus delivery has proven effective, the skilled person is well aware of other suitable delivery systems, including viral and non-viral systems.

Indeed, adenoviral vectors are known to incite a significant inflammatory response and thus are not optimal clinical vehicles for gene therapy/editing. In the present study, we evaluate AAV9-mediated in utero and postnatal intravascular delivery of an ABE to correct the G→A mutation and rescue the multi-organ disease phenotype in the Idua-W392X MPS-IH mouse model which recapitulates W402X MPS-IH disease in humans⁴².

### Definitions

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

The phrase “base editing” refers to a form of genome editing that enables direct, irreversible conversion of one base pair to another at a target genome locus without requiring double strand breaks (DSBs), homology directed repair (HDR) processes or donor DNA templates. Compared with standard genome editing methods to introduce point mutations, base editing can proceed more efficiently, and with far fewer undesired products, such as stochastic insertions or deletions (indels) or translocations.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

A monogenic disease or a monogenic disorder is a condition determined by the interaction of a single pair of genes. This is in contrast to a polygenic condition wherein several genes are involved. In humans, monogenic diseases occur less frequently than the polygenic disease. It is also less complicated than the latter and may follow a pattern based on Mendelian inheritance. Monogenetic disorders can adversely impact a number of biological systems. For example, monogenic hematopoietic stem cell disorders include, Sickle cell disease, Alpha Thalassemia, Beta Thalassemia and Fanconi Anemia. Lung disorders include Cystic Fibrosis, Surfactant Protein deficiencies and Alpha-1 anti-trypsin deficiency. In preferred embodiments of the invention, Lysosomal Storage Diseases are treated. These include Pompe Disease, Gaucher disease, Fabry Disease, Nieman Pick Disease and Batten Disease. Ornithine Transcarbamylase Deficiency, Krabbe disease, Spinal muscular atrophy, Fragile X disease, Angelman’s syndrome, Genetic epilepsies, and Tyrosinemia may also be treated using the compositions and methods disclosed herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non nucleotide components. A polynucleotide may be further modified after polymerization, such as by conjugation with a labeling component.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%. 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

The phrase “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, Molecular Cloning: A Laboratory Manual, 2nd edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)).

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, Gene Expression Technology: Methods in Enzymology 185, Academic Press. San Diego, Calif. (1990). Alternatively, the recombinant expression vector can be transcribed and translated in vitro, for example using T7 promoter regulatory sequences and T7 polymerase.

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector’s control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. Particularly preferred modified versions include, without limitation, NRRH, NRTH, NRCH, xCas9, SpCas9-NG, SpCas9, SpG, SpRY, SauriCas9, SaCas9, Nme2Cas9, VRER-SpCas9, and VQR-SpCas9. These enzymes are known to those of skill in this art area. For example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, an enzyme coding sequence encoding a CRISPR enzyme is codon optimized for expression in particular cells, such as eukaryotic human cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In an aspect of the invention, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector. In a preferred embodiment of the invention the gene product is luciferase. In a further embodiment of the invention the expression of the gene product is decreased.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). Other lipid nanoparticle formulations are disclosed in 11,066,355; 11,059,807; U.S. Pat. publications 2021/0106538 and 2021/0113466.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Several different AAV serotypes have been used to advantage for transduction of mammalian cells, these include, for example AAV1, AAV2, AAV4, AAV5, AAV6, AAV7, AAV8, and AAV9 that have different tropisms for cell types of interest. Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). In certain preferred embodiments, the viral vector is a split AAV9 vector.

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be reintroduced into the human or non-human animal.

In one aspect, the invention provides for methods of modifying a target polynucleotide in a eukaryotic cell. In some embodiments, the method comprises allowing a ABE or CBECRISPR complex to bind to the target polynucleotide to effect correction of a mutation in said target polynucleotide thereby modifying the target polynucleotide, wherein the CRISPR complex comprises the ABE or CBE CRISPR enzyme complexed with a guide sequence hybridized to a target sequence within said target polynucleotide.

In one aspect, the invention provides kits containing any one or more of the elements disclosed in the above methods and compositions. In some embodiments, the kit comprises a vector system or components for an alternative delivery system such as those described above and instructions for using the kit. In some embodiments, the vector or delivery system comprises and ABE or CBE Crispr complexed with a guide strand for base editing a target nucleic acid. The kit can contain (a) a first regulatory element optionally operably linked to a tracr mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) a second regulatory element operably linked to an enzyme-coding sequence encoding said CRISPR enzyme comprising a nuclear localization sequence. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types in methods of gene therapy. An exemplary CRISPR complex comprises a CRISPR enzyme complexed with a guide sequence hybridized to a target sequence within the target polynucleotide. The guide sequence is linked to a tracr mate sequence, which in turn hybridizes to a tracr sequence.

As used herein, the term a “metabolic gene” is defined as an inherited single gene anomaly, i.e., a single gene coding for an enzyme is defective, and that defect causes an enzyme deficiency. The enzyme deficiency produces an inherited metabolic disease or disorder, of which a subtype is an inborn error of metabolism. Most single gene anomalies are autosomal recessive, i.e., two copies of the defective gene must be present for the disease or trait to develop. Non-limiting examples of metabolic disorders include glucose metabolism disorders, lipid metabolism disorders, malabsorption syndromes, metabolic brain diseases, calcium metabolism disorders, DNA repair-deficiency disorders, hyperlactemia, iron metabolism disorders, metabolic syndrome X, inborn error of metabolism, phosphorus metabolism disorders, and acid-base imbalance. Inherited metabolic diseases previously were classified as disorders of carbohydrate metabolism, amino acid metabolism, organic acid metabolism, or lysosomal storage diseases, however new inherited disorders of metabolism have been discovered and the categories have multiplied. Certain major classes of congenital metabolic diseases include disorders of carbohydrate metabolism, e.g., glycogen storage disease, glucose-6-phosphate dehydrogenase (G6PD) deficiency (resulting from a mutation in the G6PD gene); disorders of amino acid metabolism, e.g., phenylketonuria, maple syrup urine disease, glutaric acidemia type 1; urea cycle disorder (Urea Cycle Defects), e.g., carbamoyl phosphate synthetase I deficiency; disorders of organic acid metabolism (organic acidurias), e.g., alcaptonuria, 2-hydroxyglutaric acidurias; disorders of fatty acid oxidation and mitochondrial metabolism; e.g., medium-chain acyl-coenzyme A dehydrogenase deficiency (often called “MCADD”) (caused by mutations in the ACADM gene, which results in medium-chain fatty acids not being metabolized properly and leads to lethargy and hypoglycemia); disorders of porphyrin metabolism, e.g., acute intermittent porphyria; disorders of purine or pyrimidine metabolism, e.g., Lesch-Nyhan syndrome (caused by mutations in the hypoxanthine phosphoribosyltransferase 1 (HPRT1) gene and is inherited in an X-linked recessive manner; disorders of steroid metabolism, e.g., lipoid congenital adrenal hyperplasia, congenital adrenal hyperplasia; disorders of mitochondrial function, e.g., Kearns-Sayre syndrome; disorders of peroxisomal function, e.g., Zellweger syndrome (caused by mutations in genes encoding peroxins, e.g., PEX1, PEX2, PEX3, PEX5, PEX6, PEX10, PEX12, PEX13, PEX14, PEX16, PEX19, or PEX26 genes); lysosomal storage disorders, e.g., Gaucher’s disease (of which there are three subtypes, all of which are autosomal recessive) and Niemann-Pick disease (has an autosomal recessive inheritance pattern; Niemann-Pick types A and B are caused by a mutation in the Sphingomyelin phosphodiesterase 1 (SMPD1) gene; mutations in NPC1 gene or NPC2 gene cause Niemann-Pick disease, type C (NPC), which affects a protein used to transport lipids; Niemann-Pick type D shares a specific mutation in the NPC1 gene, patients having type D shared a common Nova Scotian ancestry).

In certain aspects, an adenine base editor (ABE) complex for programming conversion of adenine to guanine in a patient in need thereof is provided where the patient has a target DNA molecule harboring a mutation associated with a lysosomal storage disease. An exemplary ABE complex includes a modified TadA enzyme, a catalytically impaired Cas 9 protein and a single guide RNA (sgRNA) which directs said ABE complex to said mutated target DNA molecule, which upon contact converts adenine in said mutation to inosine, thereby catalyzing an A-T to G-C transition following DNA replication.

When the ABE complex is directed to the correction of a W402X mutation described above associated with Hurler syndrome, the target base position to be edited is italicized and the complex and protospacer and PAM sequences can be selected from SEQ ID NOS: 1-8 shown below.
i) spCas9.ABEmax and GCTCTAGGCCGAAGTGTCGC AGG;
ii) spCas9.ABEmax and TAGGCCGAAGTGTCGCAGGC and CGG;
iii) Nme2Cas9.ABEmax and GAGCAGCTCTAGGCCGAAGTGTCG and CAGGCC;
iv) Nme2Cas9.ABEmax and CTCTAGGCCGAAGTGTCGCAGGCC and GGGACC;
v) SpG.ABEmax CTCTAGGCCGAAGTGTCGCA and GGCC;
vi) SpRY.ABEmax CTCTAGGCCGAAGTGTCGCA and GGCC;
vii) SauriCas9.ABEmax AGCTCTAGGCCGAACTCTCG and CAGG; and
viii) NRCH.ABEmax GCAGCTCTAGGCCGAAGTGT and CGCA. In certain embodiments, the ABE complex can be ABE7.10 where the guide strand comprises a protospacer and PAM sequence of 5′GCTCTAGGCCGAAGTGTCGCAGG3′ (SEQ ID NO: 1). The correct codon has been corrected, restoration of Idua activity occurs and ameliorates symptoms of Hurler syndrome.

The invention provides methods for in utero genome editing. In one approach the method can comprise administering to a patient an ABE complex as described above which introduces a modified codon into said gene sequence and corrects said mutation, wherein the base editing does not induce double strand breaks in the target nucleic acid and said correction of said mutation ameliorates symptoms of said metabolic disorder, e.g., lysosomal storage disease. In another approach a CBE complex can be administered to a subject in an adenoviral vector for example, the adenoviral vector comprising CRISPR-mediated base editor 3 (BE3) and a guide RNA (gRNA), the gRNA targeting a mutation in a therapeutic gene; and introducing a modified codon in the therapeutic gene by base editing the therapeutic gene, wherein the base editing is performed by the adenoviral vector. Base editing addresses the disadvantage of the need to create double strand breaks (DSBs) to instigate NHEJ or HDR. The base editor can comprise a catalytically impaired Streptococcus pyogenes Cas9 (SpCas9) protein, unable to make DSBs, fused to either a cytosine deaminase domain from a nucleic acid-editing protein (CBE) or a modified tRNA adenosine deaminase (ABE). The SpCas9 and gRNA tether the base editor at the genome target site, and the cytosine deaminase converts a nearby cytosine into uracil and, ultimately, thymine (resulting in either C→T or G→A changes in the coding sequence of a gene, depending on which strand is targeted). The cytosine deaminase can introduce nonsense mutations in a site-specific fashion. Alternatively, the adenine deaminase converts a nearby adenine into inosine and, ultimately, guanine and can correct a disease-causing G→A mutation. Unlike HDR, base editing does not require proliferating cells to efficiently introduce mutations.

In various embodiments, the herein provided genome editing is performed in utero when the fetus is inside a uterus of its carrier and the uterus is inside the body of a living carrier. In embodiments, the carrier may be a mammal. In certain embodiments, the mammal may be an animal. In additional embodiments, the mammal may be human. The living carrier may be the mother of the fetus or a surrogate.

In further embodiments, genome editing is performed in utero when the fetus is inside a uterus, and the uterus is outside the body of a carrier, e.g., not within the body of any living carrier, for example the uterus is in vitro or in an alternate embodiment, the uterus is ex vivo.

A method for treating a genetic disease in a fetal subject using a CBE is also disclosed. An exemplary method can comprise: identifying in vitro a target codon for base editing; generating the adenoviral vector by cloning BE3-encoding gene, a synthetic polyadenylation sequence from pCMV-BE3, CAG reporter from pCas9_GFP, and U6 promoter-driven gRNA cassette with a protospacer sequence into a dual-expression vector; administering to the fetal subject an adenoviral vector, the adenoviral vector comprising CRISPR-mediated base editor 3 (BE3) and a guide RNA (gRNA), and the gRNA targeting a mutation in a therapeutic gene; and introducing a modified codon in the therapeutic gene by base editing the therapeutic gene, wherein the base editing is performed by the adenoviral vector.

In alternate embodiments, the CRISPR-mediated base editor is base editor 4 (BE4) instead of BE3. In certain embodiments, the target codon is screened for a glutamine residue and a tryptophan residue, wherein the glutamine and tryptophan residues are within a base editing window of a protospacer adjacent motif (PAM) of the BE3, wherein the window is flanked by four proximal and four distal bases, wherein the proximal and distal bases match reference sequences.

In certain embodiments, the base editing occurs prior to disease onset, wherein the disease is a phenotype resulting from the mutation in the therapeutic gene. The base editing may decrease a risk of developing a disease.

In some embodiments, the herein provided method for treating a genetic disease in a fetal subject via genome editing is performed in utero wherein the fetus is inside a uterus of its carrier and the uterus is inside the body of a living carrier. In other embodiments, the carrier may be a mammal. In certain embodiments, the mammal may be an animal. In additional embodiments, the mammal may be human. The living carrier may be the mother of the fetus or a surrogate.

In a preferred embodiment, the therapeutic gene is an Idua gene. In these embodiments, the introduction of a modified codon in the Idua gene, ameliorates symptoms of Hurler syndrome.

In some embodiments, the therapeutic gene is base edited in an embryo, wherein prior to implantation of the embryo in a subject, wherein the embryo is in vitro fertilized.

The following materials and methods are provided to facilitate the practice of the present invention.

### METHODS

### Selection of Guide RNAs

The gRNA targeting the loxP sites flanking the mT gene in R26m^{TmG/+} mice was selected based on our previous publication³⁷ and its predicted high on-target efficiency and low off-target effects as determined by the online tool CRISPOR⁵³. The loxP targeting protospacer and PAM was S′-ATTATACGAAGTTATATTAA|GGG-3′ (SEQ ID NO: 22). The gRNA for the Idua gene was selected following visual inspection of the sequence at the site of the G→A W392X mutation which identified an AGG PAM and protospacer in which the target adenine is at position 5. Specifically, the Idua targeting protospacer and PAM was 5′-ACTCTAGGCAGAGGTCTCAAIAGG-3′ (SEQ ID NO: 10).

### Generation of AAV Vectors

AAV9 serotype vectors containing SpCas9 and the loxP targeting gRNA (for mTmG studies) or ABEmax and the Idua targeting gRNA (for MPS-IH studies) were generated by Vector Biolabs (Malvern, PA). Due to the size of the ABE and SpCas9 and the limited packaging capacity of AAV, a split AAV intein-mediated approach was used such that half of the ABE or SpCas9, the gRNA, and intein transgenes were delivered in one AAV and a second AAV was used to deliver the corresponding intein and other half of the ABE or SpCas9 transgenes, as previously described^{43,54}.

Sequences for the framework of the split-intein SpCas9 were a kind gift as described in Truong et al.⁵⁴. Briefly, the CAG promoter driving the SpCas9 to Glu573 fused to the N-terminal Nostoc punctiforme (Npu) DnaE intein was replaced with the CMV chimeric intron promoter from pCI (Promega, Madison, WI) using standard molecular cloning techniques. Following the polyadenylation signal, the gRNA cassette—a kind gift from Kiran Musunuru (Addgene Plasmid #64711)-containing the loxp targeting protospacer sequence (5′-ATTATACGAAGTTATATTAA-3′ (SEQ ID NO: 23) was inserted with the plasmid designated as pAAV-CMV-SpCas9-N-sgloxp. The corresponding C-terminal Npu intein fused to the remaining SpCas9 sequences under the CAG promoter was inserted in the pZac backbone—a kind gift from the Gene Therapy Program, University of Pennsylvania—and was designated pAAV-CAG-SpCas9-C.

The pCMV _ABEmax plasmid (Addgene plasmid #112095), a kind gift from David Liu, served as the template for the split-intein ABE. Using Q5 High Fidelity DNA Polymerase (NEB, Ipswich, MA) amplified N- and C-termini of the ABEmax were ligated at the SpCas9 Glu573 and Cys574 to the codon optimized N- and C-termini of the Npu intein, respectively. To simplify ligation, codons for Leu564 and Lys565 were altered to an AflII site (CTT|AAG). Those sequences were inserted between AAV ITRs in a plasmid backbone containing the CBh promoter⁵⁵ and WPRE3-bGH polyadenylation signal⁵⁶ and designated pAAV-CBh-ABEmax-N and pAAV-CBh-ABEmax-C. The U6 cassette containing the mouse IDUA protospacer sequence (5′-ACTCTAGGCAGAGGTCTCAA-3′; SEQ ID NO: 24) was inserted with the plasmid now designated pAAV-CBh-ABEmax-C-sgmIDUA.

Prior to submission of transfer plasmids to Vector Biolabs for production and purification of high titre serotype 9 AAVs, all plasmids were sequenced by the Children’s Hospital of Philadelphia Nucleic Acid Core or University of Pennsylvania Sequencing Facility.

### Animals

Balb/c (stock #000651), C57BL/6J (called B6; stock #000664), B6.129(Cg)-Gt(ROSA)26Sor^{tm4}(^{ACTB-tdTomato,-EGFP})^{Luo}/J (called R26^{mTmG/+}; stock #007676), and B6.126S-Idua^{tm1.1Kmke}/J (called Idua-W392X, stock #017681) mice were purchased from The Jackson Laboratory (Bar Harbor, ME). Animals were housed in the Laboratory Animal Facility of the Colket Translational Research Building at The Children’s Hospital of Philadelphia (CHOP). The experimental protocols were approved by the Institutional Animal Care and Use Committee at CHOP and followed guidelines set forth in the National Institutes of Health’s Guide for the Care and Use of Laboratory Animals.

### Genotyping

Idua-W392X mice were genotyped to confirm homozygosity for the G→A (W392X) mutation in the Idua gene and homozygotes were subsequently bred for colony maintenance/expansion and time dated experiments for in utero injections. 2-mm tail snips were digested using Lucigen Quick Extract DNA Solution (Lucigen, Middleton, WI) as per kit instructions. Extracted DNA was amplified using primers Idua-F (5′-TGCTAGGTATGAGAGAGCCA-3′;SEQ ID NO: 25) and Idua-R (5′-AGTGTAGATGAGGACTGTGGT-3′;SEQ ID NO: 26) and the PCR product was analyzed by Sanger sequencing to confirm homozygosity at the mutation site.

### In Utero and Postnatal Mouse Injections

Intravenous in utero injections were performed as previously described³⁷. Fetuses of time-dated R26^{mTmG/mTmG} × Balb/c (to generate R26m^{TmG/+} fetuses) and Idua-W392X mice were injected on gestational day (E) 15.5. Under isoflurane anaesthesia and after providing local anaesthetic (0.25% bupivacaine subcutaneously), a midline laparotomy was performed, and the uterine horn was exposed. The vitelline vein—which runs along the uterine wall and enters the portal circulation resulting in first-pass effect to liver and systemic delivery via the ductus venosus-was identified under a dissecting microscope and 15 µL of total virus (7.5 µL of each split-intein AAV vector) was injected per fetus using a 100-µm beveled glass micropipette. Based on the viral titres (Table 1), this resulted in the injection of 6.5x10¹⁰ total genome copies for Idua-W392X fetuses injected with AAV.ABE.Idua and 2x10⁹ genome copies for R26m^{TmG/+} fetuses injected with AAV. SpCas9.mTmG. A successful injection was confirmed by temporary clearance of blood from the vein and absence of injectate extravasation. The uterus was returned to the abdominal cavity and the laparotomy incision was closed in two layers with 4-0 Vicryl suture.

TABLE 1

| | |
|---|---|
| Split AAV | Titre (GC/mL) |
| AAV.ABE W392X C-terminus | 2.8×10¹² |
| AAV.ABE W392X N-terminus | 5.8×10¹² |
| mTmG C-terminus | 1.5×10¹¹ |
| mTmG N-terminus | 1.2×10¹¹ |

Viral injections into adult Idua-W392X mice were performed at 10 weeks-of-age via the retroorbital vein under isoflurane anaesthesia. A total volume of 300 µL of virus (150 µL of each split-intein AAV vector) was injected such that ~1.3x10¹² genome copies were injected per mouse. Based on the average weight of 10 week-old Idua-W392X mice (20 grams) and E15.5 fetuses (1 gram), both adult and fetal Idua-W392X mice received ~6.5x10¹⁰ total genome copies/gram.

### R26m^{TmG/+} Studies

R26m^{TmG/+} fetuses were injected via the vitelline vein at E15.5 with AAV.SpCas9.mTmG. Injected mice were sacrificed at DOL1 and 7 at which time organs, including the heart and liver were assessed for GFP expression indicative of on-target editing by flow cytometry and immunohistochemistry. Uninjected age-matched R26m^{TmG/+} served as controls.

### Idua-W392X Studies

Prenatal experiments: Idua-W392X homozygous fetuses were injected via the vitelline vein at E15.5 with AAV.ABE.Idua. In an initial screening experiment, two injected fetuses were sacrificed at 1 month-of-age at which time DNA from brain, heart, lung, liver, kidney, spleen and gonads was assessed by Sanger sequencing and NGS for on-target editing to correct the G→A (W392X) mutation. A separate cohort of mice was set up for long-term genetic, metabolic and phenotypic studies. Specifically, E15.5 Idua-W392Xhomozygous fetuses were injected at E15.5 with AAV.ABE.Idua and maintained until 6 months-of-age, the designated study endpoint. Uninjected age- and sex-matched Idua-W392X mice and wild-type B6 mice served as positive and negative controls, respectively. Urine was collected monthly starting at 1 month-of-age for GAG analysis. Serum was collected at 1 month-of-age for immunologic studies (see below) and at 6 months-of-age for IDUA activity analysis. Echocardiography was performed at 4 months-of-age and just prior to sacrifice (6 months-of-age). Additionally, at 6 months-of-age, a microCT (µCT) of the entire mouse body was performed and mice were subjected to a repetitive open field test and a grip strength test. Following sacrifice at 6 months-of-age, DNA from brain, heart, lung, liver, kidney, spleen and gonads was assessed by Sanger sequencing and NGS for off-target editing and on-target editing to correct the G→A (W392X) mutation. Immunohistochemistry was also performed. Finally, organs were assessed for GAG content and IDUA activity.

Postnatal experiments: For adult Idua-W392X studies, mice were injected via the retroorbital vein at 10 weeks-of-age with AAV.ABE.Idua. Urine was collected for GAG analysis prior to injection and at 4 and 5 months-of-age. Serum was collected for immunologic studies at 1-month post-injection. Echocardiography was performed at 4 months-of-age as were 10 mm² liver biopsies from which genomic DNA was assessed by NGS for on-target editing to correct the G→A (W392X) mutation and from which liver IDUA activity and GAGs were evaluated.

### On-target and Off-target Sequence Analysis

On-target editing of the Idua gene was assessed by Sanger sequencing and NGS. Genomic DNA was extracted from the indicated tissue using the Qiagen DNEasy Blood and Tissue Kit according to the manufacturer’s instructions (Qiagen, Hilden, Germany). Q5 polymerase was used to amplify the genomic region encompassing the W392X mutation with an annealing temperature of 66° C. PCR products were assessed using a 1% agarose gel and then purified using the Qiagen PCR Purification Kit according to the manufacturer’s recommendations. The top ten off-target sites were predicted using CRISPOR (http://crispor.tefor.net), ranked using the Cutting Frequency Determination (CFD) off-target score, amplified using Platinum SuperFi II Hi-Fidelity DNA Polymerase (Thermo Fisher Scientific, Waltham, MA) with a universal annealing temperature of 60° C., and similarly purified and evaluated by NGS for the target genomic regions. Sanger and NGS were conducted by GeneWiz, Plainfield, NJ and data from NGS was analysed using CRISPResso2⁵⁷. For NGS studies, at least 50,000 paired-end reads for each PCR amplicon at each target site for each sample was obtained. Table 2 lists the PCR primers used for amplification and Table 3 lists the predicted Idua off-target sites.

TABLE 2

Primers used for Sanger sequencing and NGS in on- and off-target analysis

| Target | Forward Primer | Reverse Primer |
|---|---|---|
| On-target | | |
| Idua | TGCTAGGTATGAGAGAGCCA (SEQ ID NO: 25) | AGTGTAGATGAGGACTGTGGT (SEQ ID NO: 26) |
| Off-target | | |
| Intron:1700010114Rik | GGGATTGCTCTGCTCTGTCT (SEQ ID NO: 27) | TGTGTAAGAGTGGGCCATGT (SEQ ID NO: 28) |
| Intron:Wnt11 | CAGGCTTGAACACACACACA (SEQ ID NO: 29) | AAAATCCCGTTGAGACCCCA (SEQ ID NO: 30) |
| Intergenic: Papl-Fbxo27 | CAACATTTGGAAGTCTGAGGC (SEQ ID NO: 31) | TGCTGGGGTTACAAGGGTG (SEQ ID NO: 32) |
| Intergenic:Fgf9-Gm25614 | ACTGCAGGAATGGAAAACTCC (SEQ ID NO: 33) | CTCTAGAGACCCTGTGCTGG (SEQ ID NO: 34) |
| Intergenic:Gm12106-Stc2 | AGGCCTTCGATCAGACATCA (SEQ ID NO: 35) | CAACAACATGGCTGCTCAGG (SEQ ID NO: 36) |
| Intergenic:Gm26190-B3gat1 | CCTTCACTCTCTTGGGCCTT (SEQ ID NO: 37) | CAGTGTCAGCAAAGGGAAGC (SEQ ID NO: 38) |
| Intergenic:Ccdc85c-Hhipl1 | ACAAGGAGGGGTGTGTGTAC (SEQ ID NO: 39) | CTGCTGAGAGGTCCTGGAG (SEQ ID NO: 40) |
| Intron:Osbpl1a | GCCCACTTAATAACCCTGTGT (SEQ ID NO: 41) | GCAGGAGGGGTCATTGATCT (SEQ ID NO: 42) |
| Intron:Blnk | ACAGCACTGAGAAGGGACAA (SEQ ID NO: 43) | CGGGAGGGATCGTAAAGTGA (SEQ ID NO: 44) |
| Intron:Rhoj | TTGGCTAGTCTCCGTGTGAA (SEQ ID NO: 45) | GGGGTCTAGAGGTCTTTGGG (SEQ ID NO: 46) |

TABLE 3

Off-target sites for IDUA

| Protospacer and PAM | Location | CFD Off-target Score |
|---|---|---|
| ACTCTAGGCAGAGGTCTCAA AGG (SEQ ID NO: 10) | Exon:Idua | |
| GTTCTAGACTGAGGTCTCAA GGG (SEQ ID NO: 11) | Intron:1700010114Rik | 0.802139 |
| ACTCCAAGCTGGGGTCTCAA CGG (SEQ ID NO: 12) | Intron:Wnt11 | 0.637255 |
| ACTCTAGGCTAGAGTCTCAA AGG (SEQ ID NO: 13) | Intergenic:Papl-Fbxo27 | 0.588235 |
| ACTTTTGACAGAGGTATCAA GGG (SEQ ID NO: 14) | Intergenic:Fgf9-Gm25614 | 0.571429 |
| ATTCCAGCCAGAGGTATCAA AGG (SEQ ID NO: 15) | Intergenic:Gm12106-Stc2 | 0.559441 |
| AGTTCAGACAGAGGTCTCAA AGG (SEQ ID NO: 16) | Intergenic:Gm26190-B3gat1 | 0.556522 |
| GCTCCAGGCAGAGGTCCCAG GGG (SEQ ID NO: 17) | Intergenic:Ccdc85c-Hhipl1 | 0.539792 |
| GAACTAAGCAGAGGTCTCAA AGG (SEQ ID NO: 18) | Intron:Osbpl1a | 0.519481 |
| GCTCTGAGCAGAGGTCCCAA CGG (SEQ ID NO: 19) | Intron:Blnk | 0.504202 |
| ACTCTACACAGAGGTACCAA TGG (SEQ ID NO: 20) | Intron:Rhoj | 0.485294 |

### Transthoracic Echocardiography

Mice were anaesthetized with 2% isoflurane and restrained on an imaging table with electrocardiogram (EKG) sensors. Transthoracic echocardiograms were performed using the Vevo 3100 Imaging System with a linear array MX550D transducer (FUJIFILM VisualSonics, Toronto, CA). An experienced echocardiographer acquired and analysed the images using Vevo LAB analysis software. The parasternal long-axis views and short-axis views were used to assess LV function and dimensions. A modified suprasternal view was used to measure the aortic arch and the aortic annulus. Measurements were performed to evaluate aortic dimensions, wall thickness, and LV dimensions during systole and diastole of the cardiac cycle. The Vevo LAB LV analysis tool, LV trace, was used to calculate cardiac output, fractional shortening, ejection fraction, stroke volume, heart rate, LV mass, dimensions, and volumes. Aortic annulus dimensions were taken during systole at maximal separation of the aortic cusps. Aortic arch measurements were taken in the ascending aorta during systole at the widest segment proximal to the innominate artery. Both imaging and analysis took place with the operator blinded to the treatment that each subject received.

### Micro Computed Tomographic Scan (µCT)

µCTs were conducted using a Siemens Inveon Multi-modality microPET/SPECT/CT platform (Siemens, Munich, Germany). Mice were anaesthetized with 2% isoflurane and placed in the µCT chamber. Slices (34 µm) were obtained with an integration time of 100 ms and reconstructed using Inveon Acquisition Workplace. DICOM images were analysed using Dragonfly v4.1 (Object Research Systems, Montreal, CA). Morphometric parameters were obtained as follows: snout angle (midsagittal angle between the nasal bone and the hard palate); skull width (maximum width at zygomatic arch); skull length (maximum caudal-rostral distance); zygomatic arch width (maximum thickness in the xy plane). Bone parameters endorsed by the American Society for Bone and Mineral Research were obtained using semi-automated bone analysis. Regions of interest were defined manually. Bone was segmented using the Otsu method. Cortical and trabecular bone were further segmented using the Buie algorithm at a trabecular thickness of 150 µm⁵⁸. At sacrifice, handheld calipers were used to measure the midshaft femoral width and length.

### Open Field Test (OFT)

After ensuring at least 72 hours free of any research or anaesthetic exposure, OFTs were conducted in a neutrally lit quiet environment in a polycarbonate arena measuring 44 cm × 44 cm × 9 cm. After a 15-minute acclimatization period, mice were placed into the centre of the arena for 5 minutes at a time for a total of 3 trials. Video footage was acquired, cropped, and then converted to raw AVI format using FFMPEG. Videos were then analysed using the MouBeAT plugin for NIH ImageJ using a minimum detection threshold of 100 sq. pixels⁵⁹. Colour thresholding was set at 42. Rearing activity was scored manually by a blinded observer.

### Grip Strength Test

After ensuring at least 72 hours free of any research or anaesthetic exposure, grip strength was assessed using a digital force meter. Mice were removed from their cages and lifted by the tail to induce the forelimbs to grasp a 1.5 mm diameter metal bar attached to the grip strength meter. After engagement with the bar, the mouse was drawn away in the plane of the meter until its grip was broken. The maximum isometric muscular contraction was recorded. Three trials were obtained for each mouse and averaged.

### IDUA Activity

α-L-iduronidase tissue and serum assays were performed using a protocol described extensively by Ou et al⁶⁰. After organ harvest, 20 mg tissue samples were homogenized with 0.1% Triton X-100 lysis buffer using a TissueLyser LT (Qiagen, Hilden, Germany). α-L-iduronidase activity was induced in specimens (25µL of tissue homogenate or serum) using 4-methyl-umbelliferyl-α-L-iduronide (Glycosynth, Warrington, UK) in 0.4 M sodium formate buffer. After incubation for 30 minutes, reactions were arrested using glycine carbonate buffer and fluorescence measured at excitation 360 nm and emission 460 nm using a fluorescent plate reader (BioTek, Winooski, VT). A standard calibration curve was generated using 4-methylumbelliferone (Sigma-Aldrich, St. Louis, MO) with arrestant buffer at a detection sensitivity of 80. Enzyme activity was normalized to tissue lysate protein content using the Pierce BCA Protein Assay Kit according to manufacturer’s instructions (Thermo Fisher Scientific, Waltham, MA).

### Glycosaminoglycan (GAG) Measurements

After obtaining IDUA enzyme activity, 0.5 mL of papain solution was added to homogenized tissue lysates and incubated at 65° C. for 3 hours and then centrifuged to clarify supernatant (Sigma-Aldrich). The Blyscan Glycosaminoglycan Assay (Biocolour, Carrickfergus, UK) was then used according to manufacturer instructions to develop a calibration curve and measure sample absorbances. Tissue GAG content was normalized to tissue lysate protein content using the Pierce BCA Protein Assay Kit. Urine GAGs were quantified using the same kit and normalized to urine creatinine using the Mouse Creatinine Assay Kit according to manufacturer instructions (Crystal Chem, Elk Grove Village, IL).

### SpCas9 Antibody Analysis

Serum levels of anti-SpCas9 antibodies were assessed in Idua-W392X mice that were prenatal and postnatal recipients of AAV.ABE.Idua as well as uninjected 4 month old IDUA-W392X mice as previously described³⁷. Serum was harvested 1 month post-injection and antibody levels determined by ELISA. Ninety-six well Nunc MaxiSorp Plates (Thermo Fisher Scientific) were coated with SpCas9 protein (PNA Bio #CP01, Newbury Park, CA) at 0.5 µg/well in 1 × coating buffer diluted from Coating Solution Concentrate Kit (SeraCare, Milford, MA) and placed at 4° C. overnight. Plates were washed with 1 × Wash buffer and blocked with 1% BSA Blocking Solution (SeraCare) at room temperature for 1 hour. Experimental and control sera were diluted 1000-fold with 1% BSA Diluent Solution (SeraCare) and added to wells for 1 hour at room temperature with shaking. The mouse monoclonal anti-SpCas9 antibody (Clone 7A9, #A-9000-100, Epigentek, Farmingdale, NY) was serially diluted in 1% BSA Diluent Solution and used as a standard to quantify anti-SpCas9 IgG1 levels. After the 1-hour incubation, wells were washed, and 100 µL of HRP-labeled mouse IgG_{K} binding protein (#SC-516102, Santa Cruz Biotechnology, Santa Cruz, CA) was added to each well for an additional 1 hour at room temperature. Wells were subsequently washed 4 times and incubated with 100 µL of ABTS ELISA HRP Substrate (SeraCare). The SpectraMax M5 plate reader (Molecular Devices, San Jose, CA) with SoftMax Pro 6.3 software was used to measure Optical density at 410 nm.

### Histology

For standard histology, tissues were fixed at 4° C. in 4% paraformaldehyde overnight and then manually dehydrated to 70% ethanol. Embedding, sectioning, and staining for Hematoxylin Eosin, Alcian Blue and Trichrome were conducted by IHC World (Ellicott City, Maryland). Slides and specimens were imaged using a Leica DMi8 (Leica Biosystems). Quantification of Alcian Blue was conducted using built-in ImageJ algorithms for stain-specific colour deconvolution. Stain thresholding was set using the Otsu method and measured in each high-power field. Nuclei count was obtained using Renyi thresholding on binary hematoxylin channel images and then using ImageJ particle analysis with a nuclear size 100-900 µm.

For whole mount histologic analysis, antibody staining on thick tissue sections was performed as previously described⁶¹. Tissue was fixed in 2% paraformaldehyde overnight at 4° C. and washed the next day in cold PBS. The tissue was embedded in agarose, and precision cut tissue slices at 150 µm were obtained using a Compresstome vibrating microtome (Precisionary Instruments, Natick, MA). Tissue slices were prepared for whole mount immunohistochemistry first by blocking overnight in a staining buffer that included 1% bovine serum albumin in PBS with 1% Triton X-100 (PBST). Incubation of primary antibodies including rabbit anti-IDUA-c-terminal (IDUA; 1:50), rat anti-leucine-rich repeat-containing G-protein receptor 5 (LGR5; 1:50) for liver tissue sections, and rabbit anti-cardiac troponin (CTNT; 1:50) for cardiac tissue was performed in staining buffer for 48 hours. The sections were washed 6 times over 1 hour in PBST. Sections were then incubated with secondary antibodies (donkey anti-rat or anti-rabbit Alexa Fluors) in staining buffer for 48 hours. Sections were again washed for 6 hours with the last wash including DAPI. The tissue section was cleared overnight in Scale SQ5 at 37° C. and then mounted in Scale S4 for imaging with a Leica Thunder Imaging system⁶². Table 4 lists the antibodies used for histology.

TABLE 4

Antibodies used for immunofluorescence (IF), flow cytometry (FC), and flow cytometry isotype control (FC-Isotype)

| Type | Antibody | Catalog # | Clone # | (Host, Source) |
|---|---|---|---|---|
| IF | Anti-Cardiac Troponin I | PA5-28964 | Polyclonal | (rabbit, Thermo Fisher Scientific) |
| IF | Anti-LGR5 | LS C804326 | Polyclonal | (rabbit, LSBio) |
| IF | Anti-IDUA C-terminus | AB178808 | Polyclonal | (rabbit, Abcam) |
| IF | Anti-GFP | AB_2307313 | Polyclonal | (chicken, Aves Labs) |
| IF | Alexa Fluor 647 | AB150075 | Polyclonal | (donkey, Abcam) |
| IF | Alexa Fluor 488 | AB150153 | Polyclonal | (donkey, Abcam) |
| IF | Alexa Fluor 514 | A31558 | Polyclonal | (goat, Invitrogen) |
| FC | Anti-CD45-PerCP-Cyanine5.5 | 45-04541-82 | 30-F11 | (rat, eBioscience) |
| FC | Anti-CD31-Brilliant Violet 421 | 102424 | 390 | (rat, Biolegend) |
| FC | Anti-CD90.2-PE-Cyanine7 | 25-0902-82 | 53-2.1 | (rat, eBioscience) |
| FC | Anti-LGR5-PE | 130-111-201 | DA04-10E8.9 | (rat, Miltenyi) |
| FC | Anti-CD45-APC | 17-451-82 | 30-F11 | (rat, eBioscience) |
| FC-Isotype | IgG2a Kappa PE Cyanine7 | 25-4321-82 | eBR2a | (rat, eBioscience) |
| FC-Isotype | IgG2a Kappa Brilliant Violet 421 | 400535 | RTK2758 | (rat, Biolegend) |
| FC-Isotype | IgG2b Kappa PE | 553989 | A95-1 | (rat, BD Pharmingen) |

### Cardiomyocyte Isolation

Cardiomyocytes, cardiac fibroblasts, and cardiac endothelial cells were isolated from in utero AAV.ABE.Idua injected Idua-W392X mice to assess for on-target gene editing in these cell populations. For cardiomyocyte isolation, freshly dissected hearts were transferred immediately to a petri dish containing ice cold calcium free Hanks Balanced Salt Solution (HBSS). A 10 mm² section of LV was excised sharply and quartered. The Pierce Primary Cardiomyocyte Isolation Kit (Thermo Fisher Scientific) was used per the manufacturer’s instructions except as follows. Enzyme supernatant and washings were reserved and combined to isolate non-cardiomyocyte cell fractions. After removal of supernatant, the remaining digestion products contained cardiomyocytes and were homogenized using 1 mL pipette tips cut to 3-5 mm diameters and then filtered through a 250 µm tissue strainer to avoid shear damage. Serial gravity filtration on ice was then employed for up to 3 cycles of 20 minutes to enrich cardiomyocytes. Cardiomyocyte enrichment was verified via light microscopy with confirmation of a majority of sarcomere containing cells and visible contraction. Genomic DNA from the enriched cardiomyocyte population was isolated using the Quick Extract DNA Solution as per the manufacturer’s instructions. The supernatants containing the non-cardiomyocyte cell fractions were subjected to flow cytometry sorting to isolate cardiac fibroblasts and endothelial cells (see below).

### Flow Cytometry

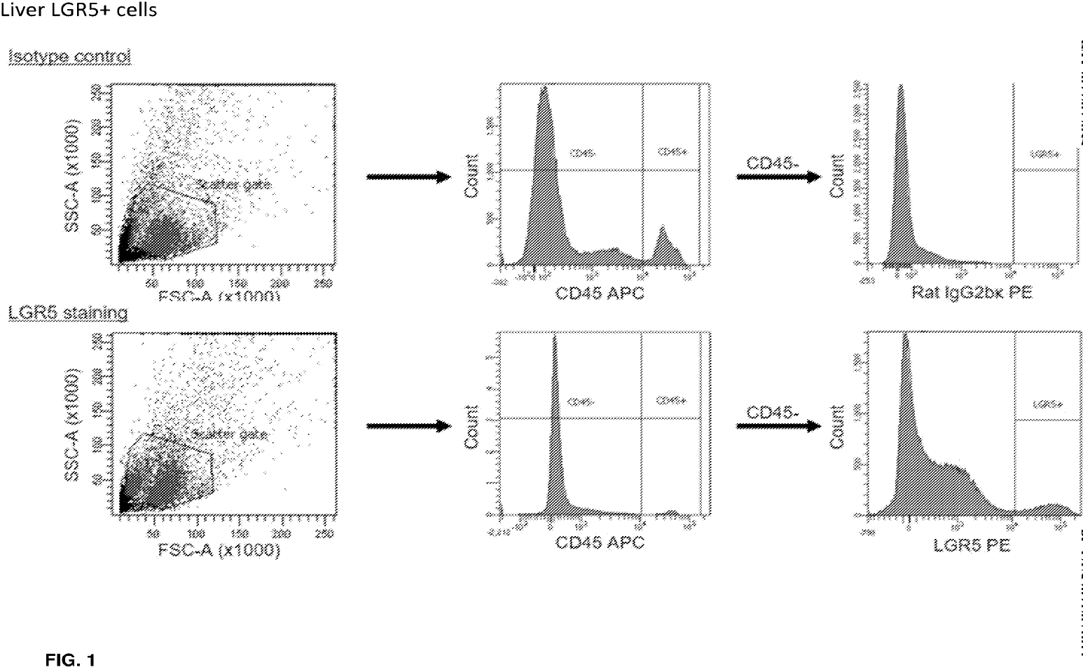

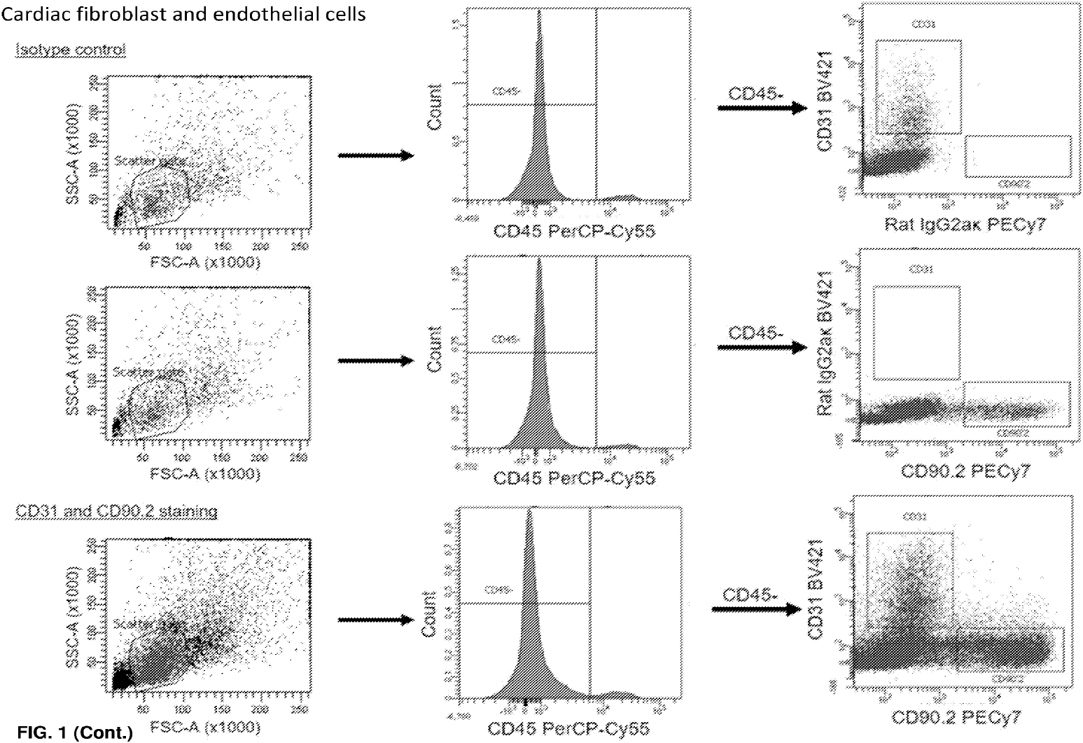

Sorting liver and cardiac cell populations: Flow cytometry was used to sort liver and cardiac cell populations from 6-month-old AAV.ABE.Idua in utero injected Idua-W392X mice. For heart, after isolation of the enriched cardiomyocyte population (described above), the remaining supernatant was centrifuged at 300 × g for 5 minutes. Pellets were resuspended in FACS staining buffer and then stained with anti-CD45-APC, anti-CD90.2-PE-Cyanine7, and anti-CD31-Brilliant Violet 421. BD FACS Aria (BD Biosciences, Franklin Lakes, NJ) was utilized to sort fibroblasts (CD45⁻ CD90.2⁺), endothelial cells (CD45⁻ CD31⁺), and bone marrow-derived cells (CD45⁺). For liver cells, freshly dissected livers were transferred immediately to a petri dish containing ice cold PBS, manually homogenized to create a single cell suspension, and resuspended in FACS staining buffer. Liver cells were subsequently stained with anti-CD45-APC and anti-LGR5-PE and then sorted into CD45⁺LGR5⁻ (hematopoietic cells) and CD45⁻LGR5⁺ (liver progenitor cells). All sorted cell populations were then lysed using QuickExtract DNA Extraction Solution to isolate genomic DNA which was then analysed by NGS for on-target Idua editing (as described above). Table 4 provides the list of antibodies used and FIG. 1 is representative flow cytometry gating for sorting liver LGR5+ cells and cardiac fibroblasts and endothelial cells.

mTmG studies: E15.5 R26m^{TmG/+} fetuses were injected with AAV9.SpCas9.mTmG, and hearts and livers were harvested at DOL1 and 1 week-of-age. Hearts and livers were processed as described above to obtain single cell suspensions. Cells were stained with DAPI to exclude dead cells and with anti-CD45-PerCP-Cyanine5.5 to exclude lymphohematopoietic cells. The expression of endogenous GFP upon excision of the mT gene via targeting the loxP sites was assessed as an indication of successful on-target editing. Uninjected R26m^{TmG/+} mice served as a negative control for gate placement. See Table 4 for the list of antibodies used.

### Statistics

All quantitative data was analysed in JMP 14.3 (SAS Institute, Inc., Cary, NC). Outlier analysis and normality assumptions were tested using normal quantile plots and fitting a normal distribution to the data. The Brown-Forsythe test was used to assess unequal variance between comparison groups. Two-sided Student’s t-test was used for direct comparisons between two groups. Groups with unequal variance were compared using the Wilcoxon test for multiple groups. Survival analysis was evaluated using Mantel-Cox log-rank comparison of survival curves. All comparison tests were conducted with α=0.05. Graphical output was generated using GraphPad Prism version 8.0.0 (GraphPad Software, San Diego, CA).

The following examples are provided to illustrate certain embodiments of the invention. They are not intended to limit the invention in any way.

### Example I

### In Utero Adeno-associated Virus Serotype 9 (AAV9) Delivery of an Adenine Base Editor (ABE) Targeting the Idua G➔A (W392X) Mutation in the MPS-IH Mouse Model

In utero base editing has the potential to correct disease-causing mutations before the onset of irreversible pathology. Mucopolysaccharidosis Type I (MPS-IH, Hurler syndrome) is a lysosomal storage disease (LSD) affecting multiple organs, often leading to early postnatal cardiopulmonary demise. We assessed in utero adeno-associated virus serotype 9 (AAV9) delivery of an adenine base editor (ABE) targeting the Idua G➔A (W392X) mutation in the MPS-IH mouse, corresponding to the common IDUA G➔A (W402X) mutation in MPS-IH patients. Here we show efficient long-term W392X correction in hepatocytes and cardiomyocytes and low-level editing in the brain. In utero editing was associated with improved survival and amelioration of metabolic, musculoskeletal, neurologic, and cardiac disease. This proof-of-concept study demonstrates the possibility of efficiently performing therapeutic base editing in multiple organs before birth via a clinically relevant delivery mechanism, highlighting the potential of this approach for MPS-IH and other genetic diseases.

### Results

### In Utero Split-Intein AAV Gene Editing in the R26m^{TmG/+} Mouse Model Results in Liver and Heart Editing

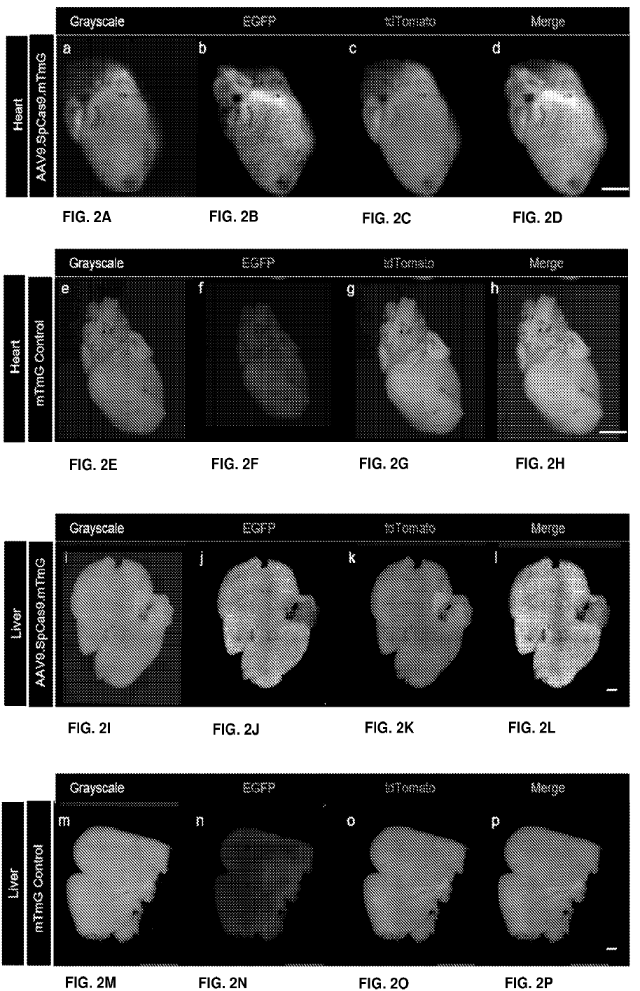

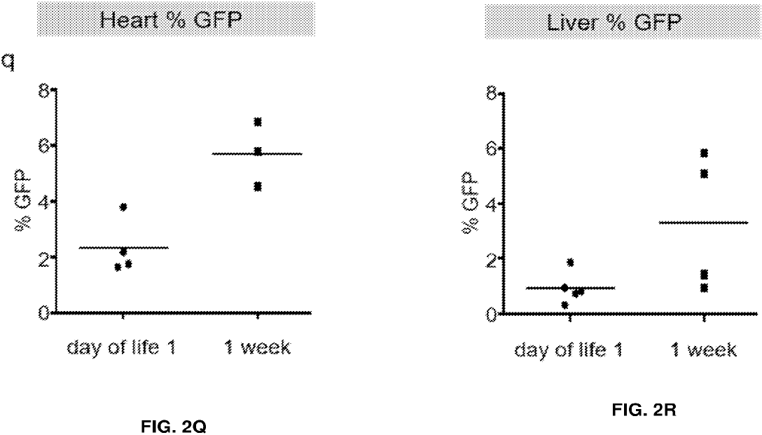

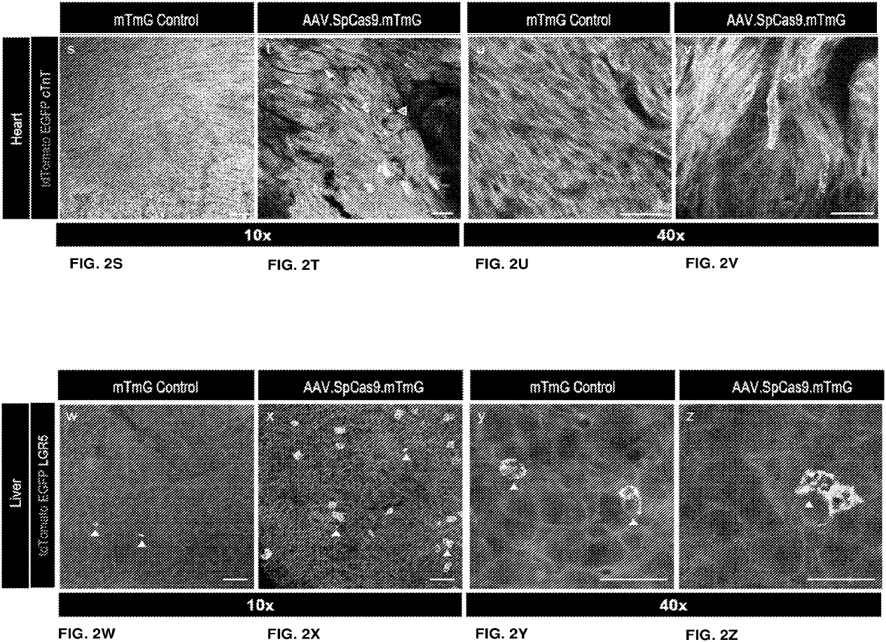

Due to the limited AAV9 packaging capacity, we sought to deliver the ABE-guide RNA (gRNA) transgene in split AAVs with reconstitution of the ABE protein in vivo via inteins^{43,44}. To initially evaluate this approach in utero, standard gene editing was performed in the tractable R26m^{TmG/+} mouse model wherein deletion of a loxP-flanked membrane tomato (mT) cassette and subsequent nonhomologous end-joining (NHEJ) switches native red fluorescence to green. Split-intein AAV9s containing SpCas9 and the loxP-targeting gRNA were injected via the vitelline vein, which drains directly into the fetal liver via the portal circulation, into embryonic day 15.5 (E15.5) R26m^{TmG/+} fetuses. Heart and liver wide-field microscopy and flow cytometry on days of life 1 and 7 demonstrated GFP expression consistent with editing (FIGS. 2A-2R). Furthermore, immunohistochemistry (IHC) to investigate discrete cell populations revealed editing in cardiomyocytes and LGR5+ liver progenitor cells (FIGS. 2S-2Z).

### In Utero Split-Intein AAV Base Editing in the Idua-W392X MPS-IH Mouse Corrects the G➔A Mutation

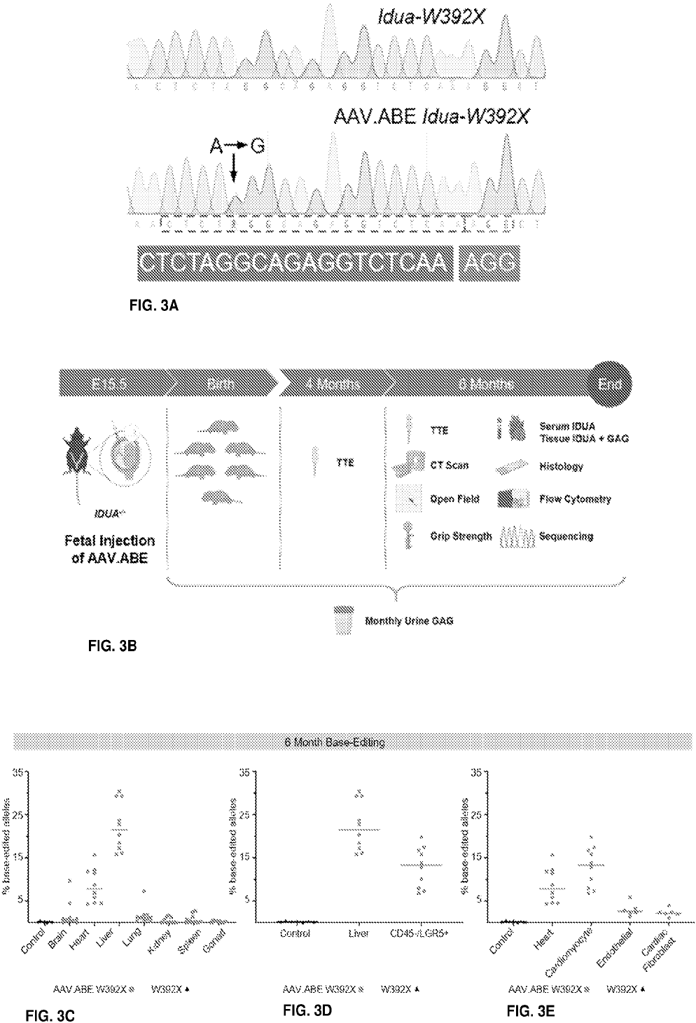

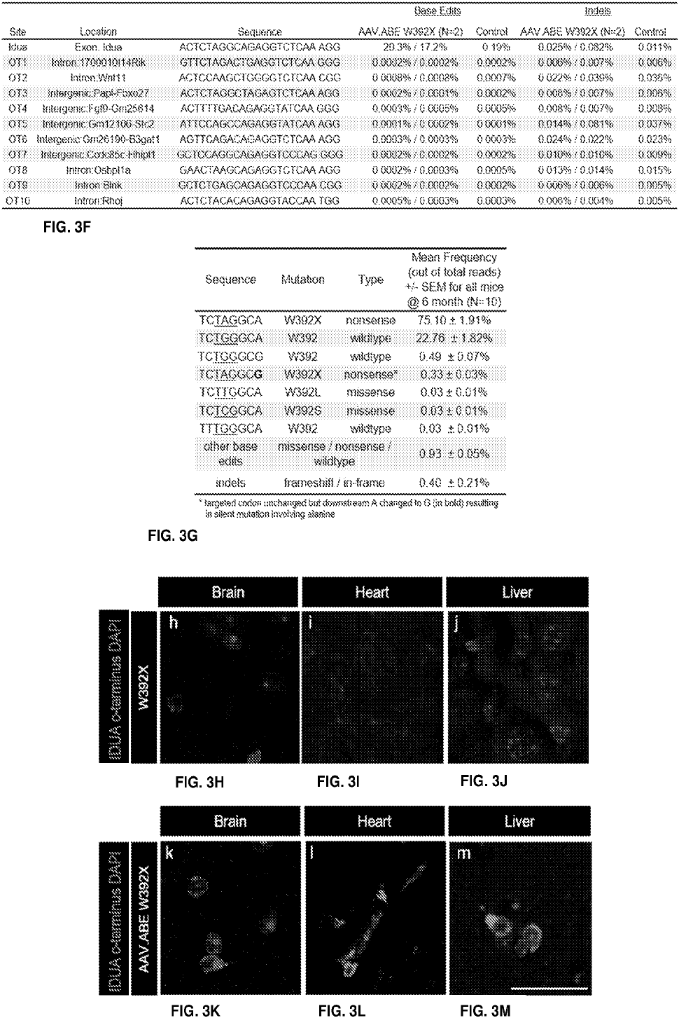

Having demonstrated successful in utero split AAV CRISPR-mediated gene editing in the heart and liver, two affected organs in MPS-IH, we next sought to use an ABE to prenatally correct the G➔A mutation in the Idua-W392X mouse model. The optimized ABE7.10 (ABEmax)⁴⁵ and gRNA with the target adenine mutation at position 5 within the 20-base protospacer sequence upstream of an NGG protospacer-adjacent motif (PAM) were packaged in two split-intein AAV9s (AAV.ABE.Idua) (FIG. 3A). In an initial screening experiment, two E15.5 Idua-W392X fetuses were injected via the vitelline vein with AAV.ABE.Idua. Sanger sequencing and next-generation sequencing (NGS) at 1 month of age demonstrated efficient heart (14.7%, 13.1%) and liver (31.1%, 22.4%) editing and low-level brain editing (1.9%, 0.66%) (FIG. 3A).

An additional cohort of E15.5 Idua-W392X fetuses was intravascularly injected with AAV.ABE.Idua for long-term genetic, biochemical, and phenotypic analyses (FIG. 3B). Uninjected age- and sex-matched Idua-W392X mice and C57BL/6 (B6) mice served as positive and negative controls, respectively. NGS at 6 months of age demonstrated efficient heart (~8.6%) and liver (~22.8%) editing in all experimental mice and low-level brain, lung, kidney, and spleen editing in some experimental mice (FIGS. 3C-3E). No gonadal editing was noted in any mice (FIG. 3C). NGS of liver genomic DNA from mice with high on-target editing did not demonstrate off-target editing above background and demonstrated low rates of nucleotide insertions or deletions (indels) and unwanted base changes (FIGS. 3F-3G). On-target editing in the heart, liver, and brain was consistent with IDUA expression observed with immunohistochemistry in these organs (FIGS. 3H-3M). Given the significant heart and liver editing, the importance of MPS-IH cardiac pathology, and the rationale that in utero editing can target progenitor cells, NGS editing efficiencies were evaluated in liver LGR5+ progenitor cells (~12.8%) and cardiac cell subpopulations including myocytes (~12.6%), endothelial cells (~3.0%), and fibroblasts (~2.3%) (FIGS. 3D-3E).

### In Utero AAV.ABE.Idua Treatment Results in Durable Biochemical Rescue in MPS-IH

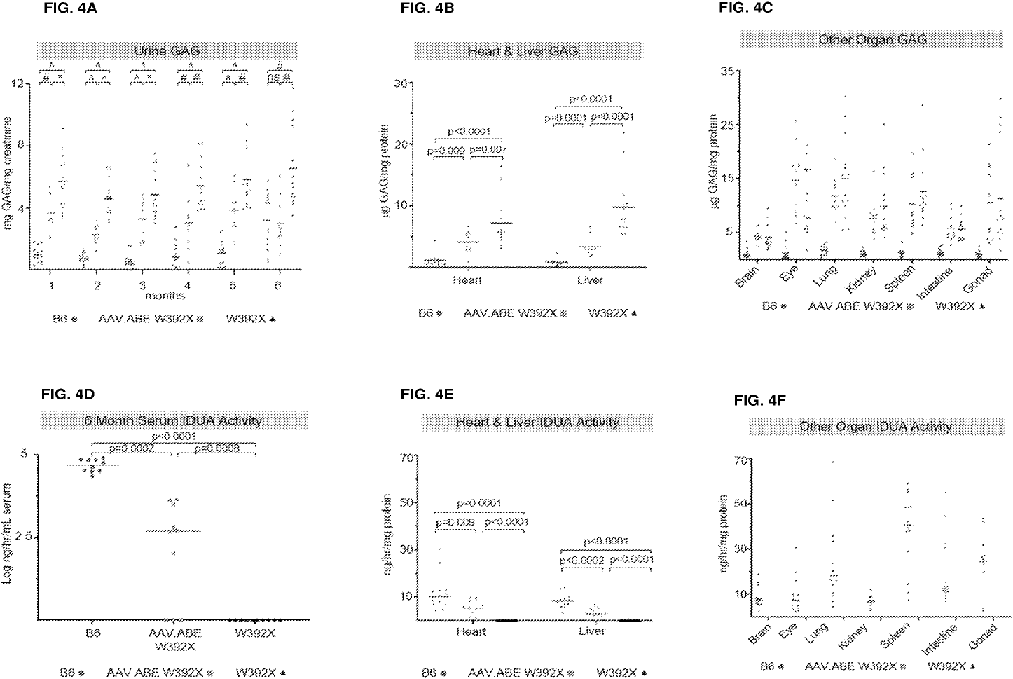

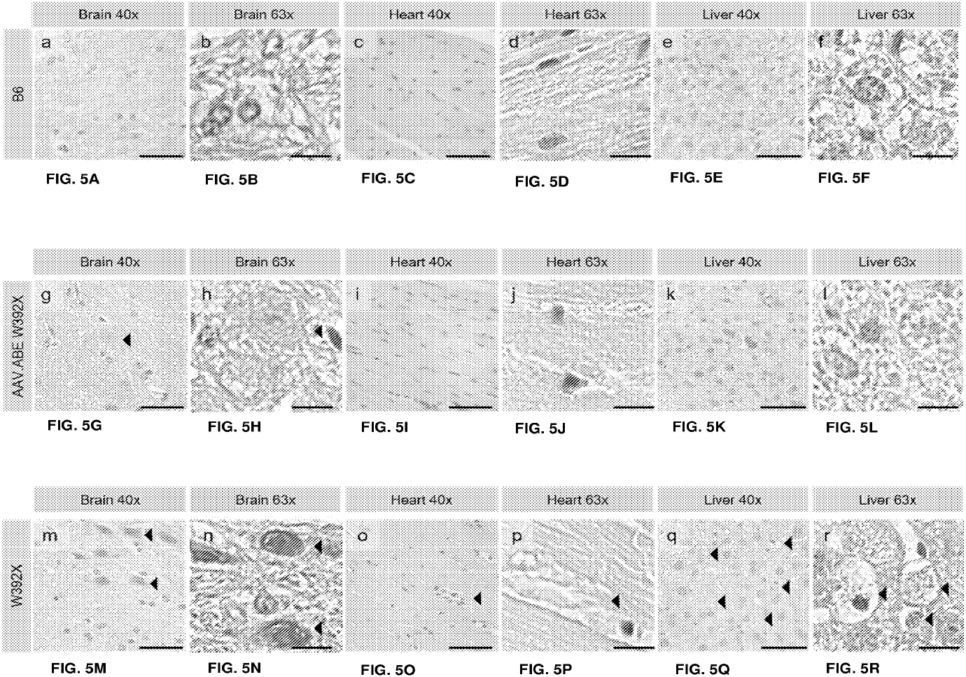

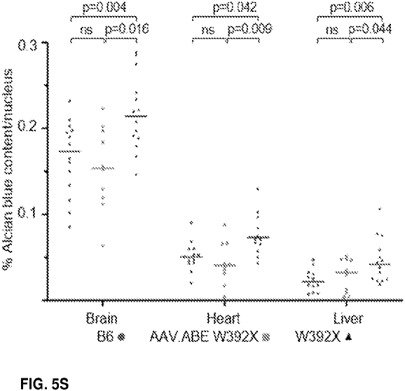

We next explored if in utero base editing caused durable biochemical corrections in Idua-W392X mice. Similar to humans with W402X MPS-IH, Idua-W392X mice have undetectable IDUA activity and increased urine and tissue GAGs⁴². In utero AAV.ABE.Idua treated mice demonstrated reduced urine GAGs as measured by colorimetric assay at all time points compared to untreated mice (FIG. 4A). At sacrifice (6 months of age), IDUA activity was increased in the serum, heart, and liver, and GAG levels were decreased in the heart and liver in AAV.ABE.Idua treated mice (FIGS. 4B-4F). Alcian blue staining of sulfated GAGs was decreased in the heart and liver, corroborating these biochemical findings, and also suggested decreased GAG accumulation in the brain (FIG. 5).

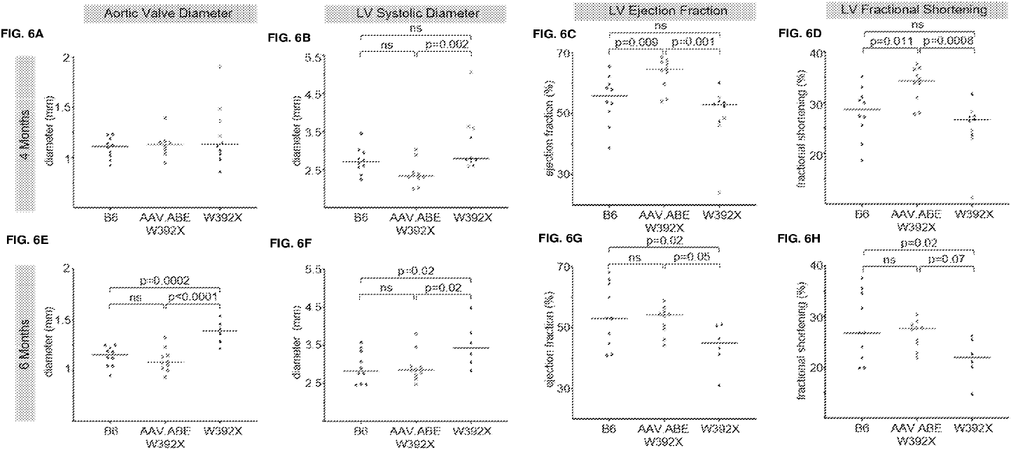

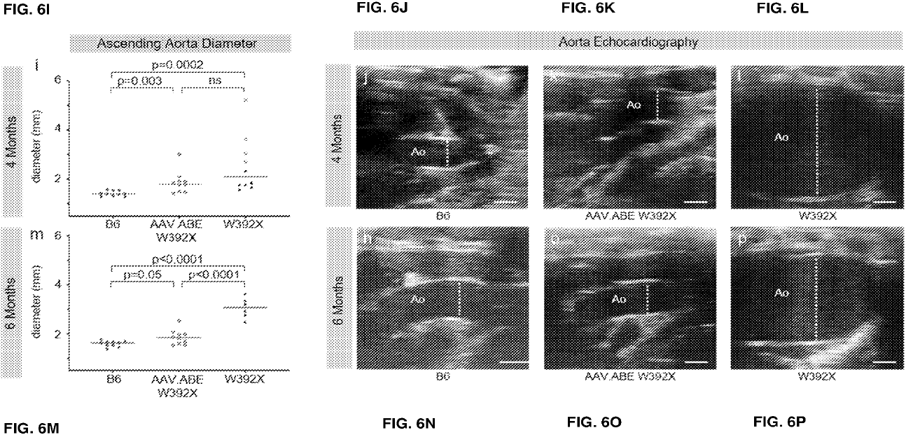

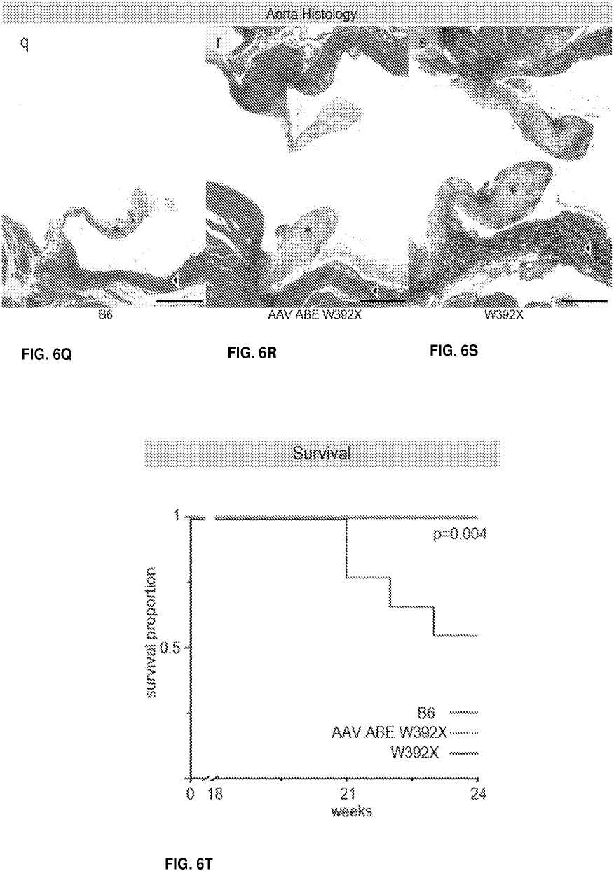

Similar to MPS-IH patients, Idua-W392X mice have a shortened lifespan⁴². In our cohort of untreated Idua-W392X mice followed for long-term studies, we observed a 6-month mortality of 40%, with non-survivors noted to have increased ascending aortic dilation and reduced left ventricular ejection fraction on 4-month echocardiography (FIGS. 6A-6D, 6I-6L). Survival of in utero treated mice was 100% and comparable to B6 mice (FIG. 6T).

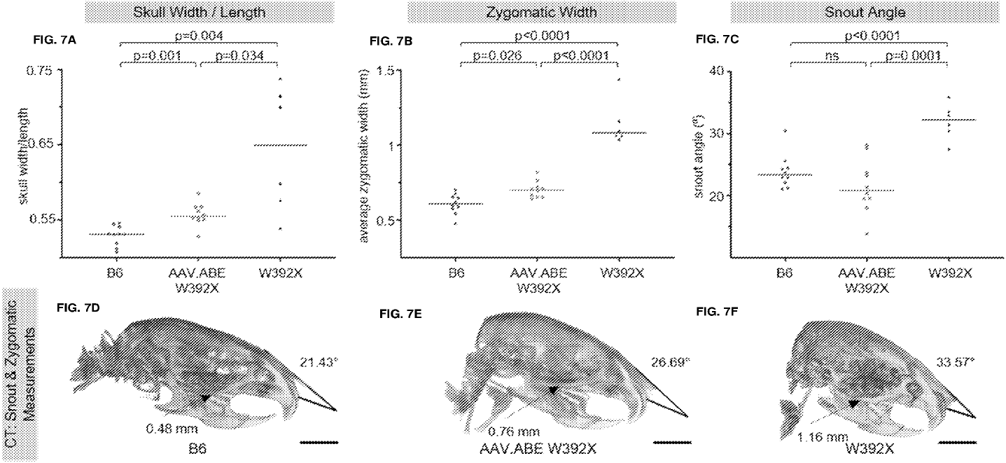

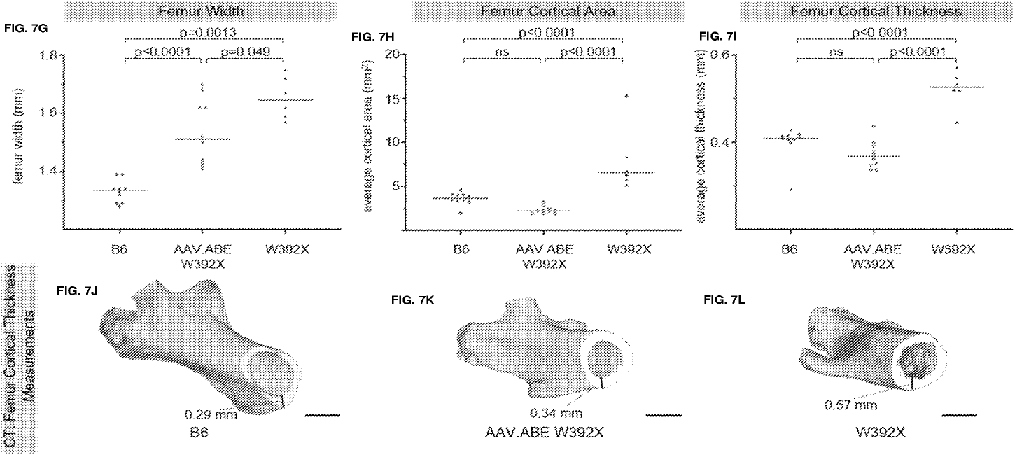

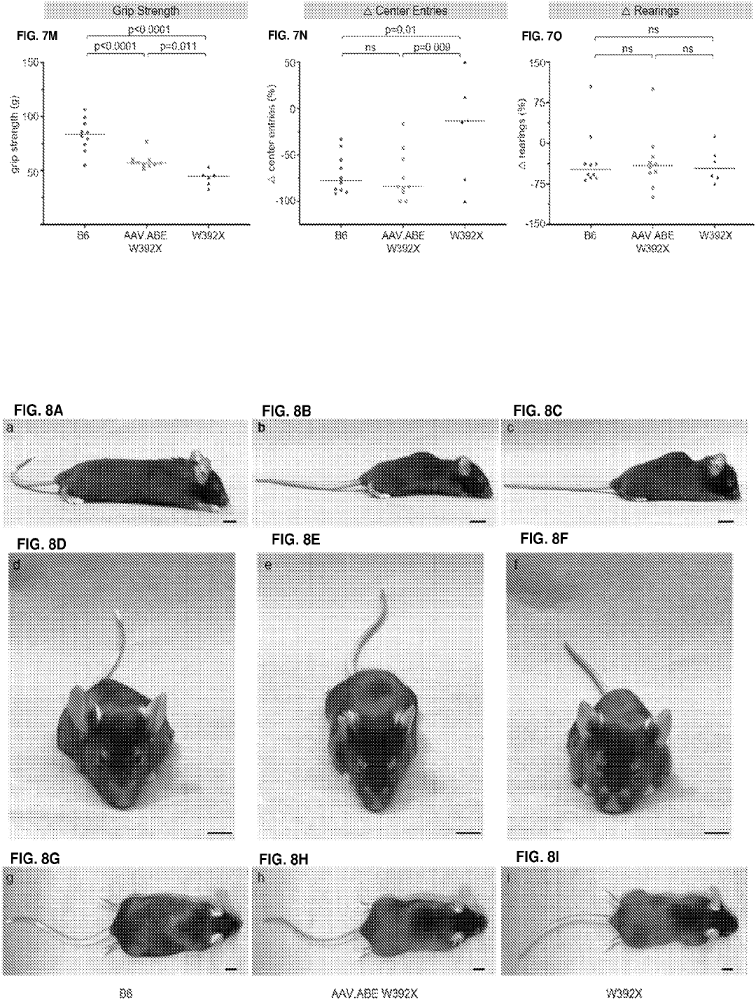

MPS-IH causes significant progressive musculoskeletal morbidity in patients requiring multiple orthopedic surgeries despite successful HSCT^{5,47-49}. Improved musculoskeletal outcomes following early versus late HSCT suggest that this irreversible pathology may benefit from in utero treatment⁴⁸. Compared to untreated Idua-W392X mice, micro-computed tomography scans demonstrated reduced skull and femur cortical bone deposition and normalized facies (FIGS. 7A-7L) in in utero AAV.ABE.Idua treated Idua-W392X mice. In addition, treated mice subjectively had decreased lordosis and snout broadness at 6 months-of-age (FIG. 8). Finally, grip strength, a test of musculoskeletal fitness, demonstrated improvement following in utero base editing (FIG. 7M).

MPS-IH patients also have severe neurocognitive deficits. Six-month-old experimental and control mice were subjected to open field testing to assess any effect of in utero base editing on this phenotype. Treated mice demonstrated reduced centre entries with repetition suggesting appropriate habituation (FIG. 7N). However, lack of improvement in other parameters such as rearing is consistent with low-level editing in the brain and emphasizes the need for strategies to enhance brain editing and/or brain IDUA activity (FIG. 7O).

### Postnatal Treatment With AAV.ABE.Idua Results in Partial Rescue of the MPS-IH Disease Phenotype but Induces an Anti-Cas9 Antibody Response

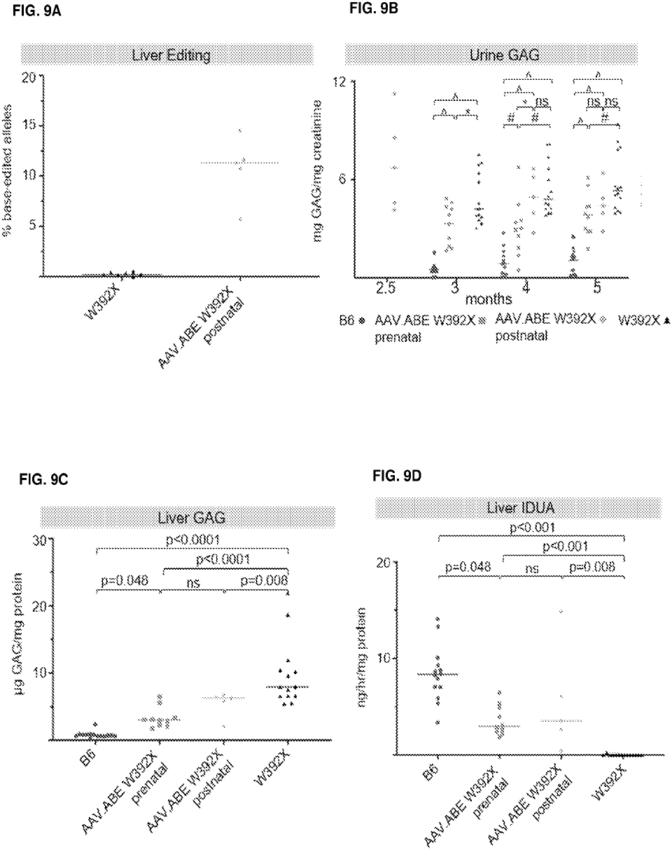

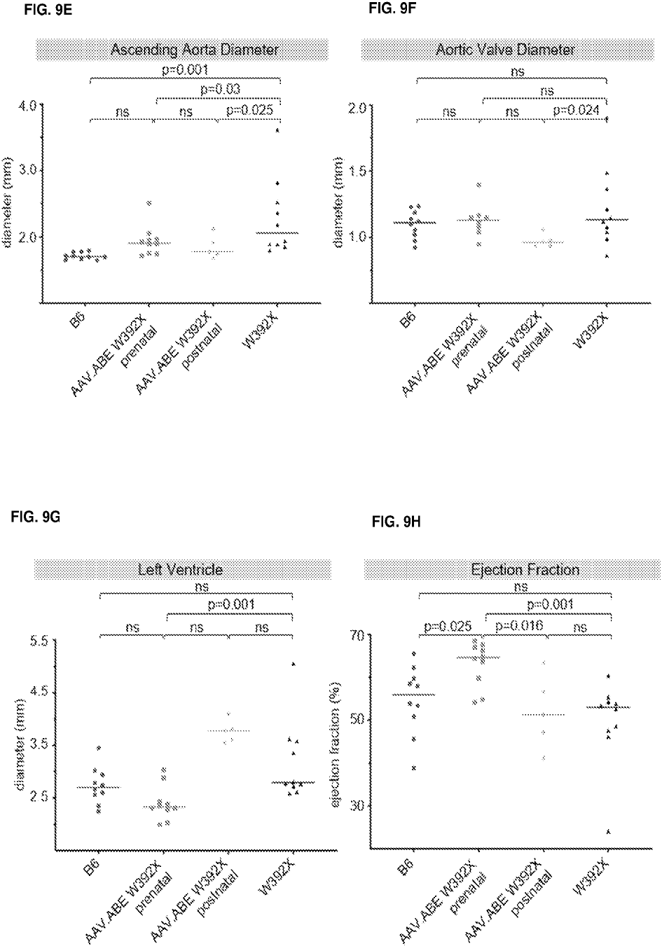

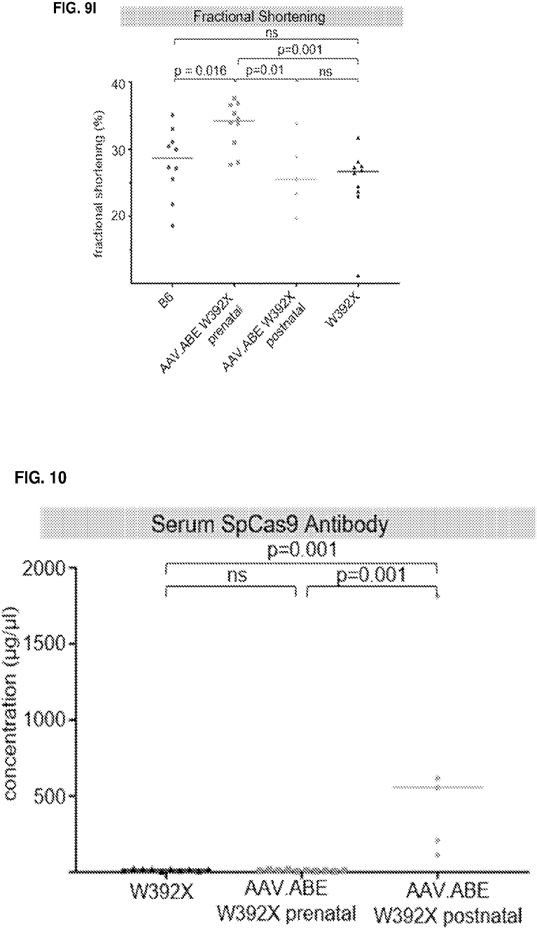

Although postnatal base editing would not prevent prenatal or early postnatal pathology, it could treat postnatally diagnosed patients. Therefore, 10-week-old immunologically mature adult Idua-W392X mice were injected via the retroorbital vein with AAV.ABE.Idua and assessed for phenotypic and biochemical changes. At 4 months of age, NGS demonstrated editing efficiencies of 5.7-11.6% in liver genomic DNA, and liver IDUA and GAG levels were significantly improved compared to untreated Idua-W392X mice (FIGS. 9A, 9C, 9D). Echocardiography revealed reduced ascending aorta and aortic valve diameters compared to untreated Idua-W392X mice but unimproved left ventricle size, ejection fraction, and fractional shortening (FIGS. 9E-9I). By 5 months of age, urine GAGs were reduced compared to untreated Idua-W392X mice (FIG. 9B). These findings suggest postnatal editing can partially correct the MPS-IH phenotype. Finally, one rationale for in utero gene editing is the absence of an immune response to the transgene product. We previously demonstrated an SpCas9 immune response following postnatal but not in utero adenoviral delivery of an SpCas9-based cytosine base editor³⁷. Adenoviral vectors are highly immunogenic, and thus we sought to determine if a similar benefit to in utero base editing exists following delivery of ABEmax via an AAV. As in the previous study, anti-SpCas9 antibodies were noted in the serum of adult but not fetal AAV.ABE.Idua recipients 1 month following injection (FIG. 10).

### References for Example I

1. Meikle PJ, Hopwood JJ, Clague AE and Carey WF. Prevalence of lysosomal storage disorders. JAMA. 1999;281:249-254.

2. Ferreira CR and Gahl WA. Lysosomal storage diseases. Transl Sci Rare Dis. 2017;2:1-71.

3. Bunge S, Kleijer WJ, Steglich C, Beck M, Zuther C, Morris CP, Schwinger E, Hopwood JJ, Scott HS and Gal A. Mucopolysaccharidosis type I: identification of 8 novel mutations and determination of the frequency of the two common alpha-L-iduronidase mutations (W402X and Q70X) among European patients. Hum Mol Genet. 1994;3:861-866.

4. Gort L, Chabas A and Coll MJ. Analysis of five mutations in 20 mucopolysaccharidois type 1 patients: high prevalence of the W402X mutation. Mutations in brief no. 121. Online. Hum Mutat. 1998; 11:332-333.

5. Tolar J and Orchard PJ. alpha-L-iduronidase therapy for mucopolysaccharidosis type I. Biologics. 2008;2:743-751.

6. Braunlin EA, Harmatz PR, Scarpa M, Furlanetto B, Kampmann C, Loehr JP, Ponder KP, Roberts WC, Rosenfeld HM and Giugliani R. Cardiac disease in patients with mucopolysaccharidosis: presentation, diagnosis and management. J Inherit Metab Dis. 2011;34:1183-1197.

7. Ahmed A, Rudser K, Kunin-Batson A, Delaney K, Whitley C and Shapiro E. Mucopolysaccharidosis (MPS) Physical Symptom Score: Development, Reliability, and Validity. JIMD Rep. 2016;26:61-68.

8. Chen MR, Lin SP, Hwang HK and Yu CH. Cardiovascular changes in mucopolysaccharidoses in Taiwan. Acta Cardiol. 2005;60:51-53.

9. Gross DM, Williams JC, Caprioli C, Dominguez B and Howell RR. Echocardiographic abnormalities in the mucopolysaccharide storage diseases. Am J Cardiol. 1988;61:170-176.

10. Martins AM, Dualibi AP, Norato D, Takata ET, Santos ES, Valadares ER, Porta G, de Luca G, Moreira G, Pimentel H, Coelho J, Brum JM, Semionato Filho J, Kerstenetzky MS, Guimaraes MR, Rojas MV, Aranda PC, Pires RF, Faria RG, Mota RM, Matte U and Guedes ZC. Guidelines for the management of mucopolysaccharidosis type I. J Pediatr. 2009;155:S32-46.

11. Hartung SD, Frandsen JL, Pan D, Koniar BL, Graupman P, Gunther R, Low WC, Whitley CB and McIvor RS. Correction of metabolic, craniofacial, and neurologic abnormalities in MPS I mice treated at birth with adeno-associated virus vector transducing the human alpha-L-iduronidase gene. Mol Ther. 2004;9:866-875.

12. Liu Y, Xu L, Hennig AK, Kovacs A, Fu A, Chung S, Lee D, Wang B, Herati RS, Mosinger Ogilvie J, Cai SR and Parker Ponder K. Liver-directed neonatal gene therapy prevents cardiac, bone, ear, and eye disease in mucopolysaccharidosis I mice. Mol Ther. 2005; 11:35-47.

13. Ou L, DeKelver RC, Rohde M, Tom S, Radeke R, St Martin SJ, Santiago Y, Sproul S, Przybilla MJ, Koniar BL, Podetz-Pedersen KM, Laoharawee K, Cooksley RD, Meyer KE, Holmes MC, McIvor RS, Wechsler T and Whitley CB. ZFN-Mediated In Vivo Genome Editing Corrects Murine Hurler Syndrome. Mol Ther. 2019;27:178-187.

14. Schuh RS, Gonzalez EA, Tavares AMV, Seolin BG, Elias LS, Vera LNP, Kubaski F, Poletto E, Giugliani R, Teixeira HF, Matte U and Baldo G. Neonatal nonviral gene editing with the CRISPR/Cas9 system improves some cardiovascular, respiratory, and bone disease features of the mucopolysaccharidosis I phenotype in mice. Gene Ther. 2020;27:74-84.

15. Kosicki M, Tomberg K and Bradley A. Repair of double-strand breaks induced by CRISPR-Cas9 leads to large deletions and complex rearrangements. Nat Biotechnol. 2018;36:765-771.

16. Yin H, Song CQ, Dorkin JR, Zhu LJ, Li Y, Wu Q, Park A, Yang J, Suresh S, Bizhanova A, Gupta A, Bolukbasi MF, Walsh S, Bogorad RL, Gao G, Weng Z, Dong Y, Koteliansky V, Wolfe SA, Langer R, Xue W and Anderson DG. Therapeutic genome editing by combined viral and non-viral delivery of CRISPR system components in vivo. Nat Biotechnol. 2016;34:328-333.

17. Yin H, Xue W, Chen S, Bogorad RL, Benedetti E, Grompe M, Koteliansky V, Sharp PA, Jacks T and Anderson DG. Genome editing with Cas9 in adult mice corrects a disease mutation and phenotype. Nat Biotechnol. 2014;32:551-553.

18. Gaudelli NM, Komor AC, Rees HA, Packer MS, Badran AH, Bryson DI and Liu DR. Programmable base editing of A*T to G*C in genomic DNA without DNA cleavage. Nature. 2017;551:464-471.

19. Huang TP, Zhao KT, Miller SM, Gaudelli NM, Oakes BL, Fellmann C, Savage DF and Liu DR. Circularly permuted and PAM-modified Cas9 variants broaden the targeting scope of base editors. Nat Biotechnol. 2019;37:626-631.

20. Jin S, Zong Y, Gao Q, Zhu Z, Wang Y, Qin P, Liang C, Wang D, Qiu JL, Zhang F and Gao C. Cytosine, but not adenine, base editors induce genome-wide off-target mutations in rice. Science. 2019;364:292-295.

21. Ryu SM, Koo T, Kim K, Lim K, Baek G, Kim ST, Kim HS, Kim DE, Lee H, Chung E and Kim JS. Adenine base editing in mouse embryos and an adult mouse model of Duchenne muscular dystrophy. Nat Biotechnol. 2018;36:536-539.

22. Lee HK, Smith HE, Liu C, Willi M and Hennighausen L. Cytosine base editor 4 but not adenine base editor generates off-target mutations in mouse embryos. Commun Biol. 2020;3:19.

23. Song CQ, Jiang T, Richter M, Rhym LH, Koblan LW, Zafra MP, Schatoff EM, Doman JL, Cao Y, Dow LE, Zhu LJ, Anderson DG, Liu DR, Yin H and Xue W. Adenine base editing in an adult mouse model of tyrosinaemia. Nat Biomed Eng. 2020;4:125-130.

24. Crawfurd M, Dean MF, Hunt DM, Johnson DR, MacDonald RR, Muir H, Wright EA and Wright CR. Early prenatal diagnosis of Hurler’s syndrome with termination of pregnancy and confirmatory findings on the fetus. J Med Genet. 1973; 10: 144-153.

25. Crow J, Gibbs DA, Cozens W, Spellacy E and Watts RW. Biochemical and histopathological studies on patients with mucopolysaccharidoses, two of whom had been treated by fibroblast transplantation. J Clin Pathol. 1983;36:415-430.

26. Ikeno T, Minami R, Wagatsuma K, Fujibayashi S, Nakao T, Abo K, Tsugawa S, Taniguchi S and Takasago Y. Prenatal diagnosis of Hurler’s syndrome-biochemical studies on the affected fetus. Hum Genet. 1981;59:353-359.

27. Weber R, Kantor P, Chitayat D, Friedberg MK, Golding F, Mertens L, Nield LE, Ryan G, Seed M, Yoo SJ, Manlhiot C and Jaeggi E. Spectrum and outcome of primary cardiomyopathies diagnosed during fetal life. JACC Heart Fail. 2014;2:403-411.

28. Endo M, Henriques-Coelho T, Zoltick PW, Stitelman DH, Peranteau WH, Radu A and Flake AW. The developmental stage determines the distribution and duration of gene expression after early intra-amniotic gene transfer using lentiviral vectors. Gene Ther. 2010; 17:61-71.

29. Stitelman DH, Brazelton T, Bora A, Traas J, Merianos D, Limberis M, Davey M and Flake AW. Developmental stage determines efficiency of gene transfer to muscle satellite cells by in utero delivery of adeno-associated virus vector serotype 2/9. Mol Ther Methods Clin Dev. 2014;1:14040.

30. Stitelman DH, Endo M, Bora A, Muvarak N, Zoltick PW, Flake AW and Brazelton TR. Robust in vivo transduction of nervous system and neural stem cells by early gestational intra amniotic gene transfer using lentiviral vector. Mol Ther. 2010;18:1615-1623.

31. Blanchette M and Daneman R. Formation and maintenance of the BBB. Mech Dev. 2015;138 Pt 1:8-16.

32. Ben-Zvi A, Lacoste B, Kur E, Andreone BJ, Mayshar Y, Yan H and Gu C. Mfsd2a is critical for the formation and function of the blood-brain barrier. Nature. 2014;509:507-511.

33. Davey MG, Riley JS, Andrews A, Tyminski A, Limberis M, Pogoriler JE, Partridge E, Olive A, Hedrick HL, Flake AW and Peranteau WH. Induction of Immune Tolerance to Foreign Protein via Adeno-Associated Viral Vector Gene Transfer in Mid-Gestation Fetal Sheep. PLoS One. 2017;12:e0171132.

34. Flotte TR, Zeitlin PL, Reynolds TC, Heald AE, Pedersen P, Beck S, Conrad CK, Brass-Ernst L, Humphries M, Sullivan K, Wetzel R, Taylor G, Carter BJ and Guggino WB. Phase I trial of intranasal and endobronchial administration of a recombinant adeno-associated virus serotype 2 (rAAV2)-CFTR vector in adult cystic fibrosis patients: a two-part clinical study. Hum Gene Ther. 2003;14:1079-1088.

35. Mingozzi F, Maus MV, Hui DJ, Sabatino DE, Murphy SL, Rasko JE, Ragni MV, Manno CS, Sommer J, Jiang H, Pierce GF, Ertl HC and High KA. CD8(+) T-cell responses to adeno-associated virus capsid in humans. Nat Med. 2007;13 :419-422.

36. Moss RB, Milla C, Colombo J, Accurso F, Zeitlin PL, Clancy JP, Spencer LT, Pilewski J, Waltz DA, Dorkin HL, Ferkol T, Pian M, Ramsey B, Carter BJ, Martin DB and Heald AE. Repeated aerosolized AAV-CFTR for treatment of cystic fibrosis: a randomized placebo-controlled phase 2B trial. Hum Gene Ther. 2007;18:726-732.

37. Rossidis AC, Stratigis JD, Chadwick AC, Hartman HA, Ahn NJ, Li H, Singh K, Coons BE, Li L, Lv W, Zoltick PW, Alapati D, Zacharias W, Jain R, Morrisey EE, Musunuru K and Peranteau WH. In utero CRISPR-mediated therapeutic editing of metabolic genes. Nat Med. 2018;24:1513-1518.

38. Sabatino DE, Mackenzie TC, Peranteau W, Edmonson S, Campagnoli C, Liu YL, Flake AW and High KA. Persistent expression of hF.IX After tolerance induction by in utero or neonatal administration of AAV-1-F.IX in hemophilia B mice. Mol Ther. 2007; 15: 1677-1685.

39. Wang D, Mou H, Li S, Li Y, Hough S, Tran K, Li J, Yin H, Anderson DG, Sontheimer EJ, Weng Z, Gao G and Xue W. Adenovirus-Mediated Somatic Genome Editing of Pten by CRISPR/Cas9 in Mouse Liver in Spite of Cas9-Specific Immune Responses. Hum Gene Ther. 2015;26:432-442.

40. Charlesworth CT, Deshpande PS, Dever DP, Camarena J, Lemgart VT, Cromer MK, Vakulskas CA, Collingwood MA, Zhang L, Bode NM, Behlke MA, Dejene B, Cieniewicz B, Romano R, Lesch BJ, Gomez-Ospina N, Mantri S, Pavel-Dinu M, Weinberg KI and Porteus MH. Identification of preexisting adaptive immunity to Cas9 proteins in humans. Nat Med. 2019;25:249-254.

41. Li A, Tanner MR, Lee CM, Hurley AE, De Giorgi M, Jarrett KE, Davis TH, Doerfler AM, Bao G, Beeton C and Lagor WR. AAV-CRISPR Gene Editing Is Negated by Preexisting Immunity to Cas9. Mol Ther. 2020.

42. Wang D, Shukla C, Liu X, Schoeb TR, Clarke LA, Bedwell DM and Keeling KM. Characterization of an MPS I-H knock-in mouse that carries a nonsense mutation analogous to the human IDUA-W402X mutation. Mol Genet Metab. 2010;99:62-71.

43. Levy JM, Yeh WH, Pendse N, Davis JR, Hennessey E, Butcher R, Koblan LW, Comander J, Liu Q and Liu DR. Cytosine and adenine base editing of the brain, liver, retina, heart and skeletal muscle of mice via adeno-associated viruses. Nat Biomed Eng. 2020;4:97-110.

44. Villiger L, Grisch-Chan HM, Lindsay H, Ringnalda F, Pogliano CB, Allegri G, Fingerhut R, Haberle J, Matos J, Robinson MD, Thony B and Schwank G. Treatment of a metabolic liver disease by in vivo genome base editing in adult mice. Nat Med. 2018;24:1519-1525.

45. Koblan LW, Doman JL, Wilson C, Levy JM, Tay T, Newby GA, Maianti JP, Raguram A and Liu DR. Improving cytidine and adenine base editors by expression optimization and ancestral reconstruction. Nat Biotechnol. 2018;36:843-846.

46. Braunlin E, Mackey-Bojack S, Panoskaltsis-Mortari A, Berry JM, McElmurry RT, Riddle M, Sun LY, Clarke LA, Tolar J and Blazar BR. Cardiac functional and histopathologic findings in humans and mice with mucopolysaccharidosis type I: implications for assessment of therapeutic interventions in hurler syndrome. Pediatr Res. 2006;59:27-32.

47. Weisstein JS, Delgado E, Steinbach LS, Hart K and Packman S. Musculoskeletal manifestations of Hurler syndrome: long-term follow-up after bone marrow transplantation. J Pediatr Orthop. 2004;24:97-101.

48. Polgreen LE, Tolar J, Plog M, Himes JH, Orchard PJ, Whitley CB, Miller BS and Petryk A. Growth and endocrine function in patients with Hurler syndrome after hematopoietic stem cell transplantation. Bone Marrow Transplant. 2008;41: 1005-1011.

49. Taylor C, Brady P, O’Meara A, Moore D, Dowling F and Fogarty E. Mobility in Hurler syndrome. J Pediatr Orthop. 2008;28:163-168.

50. Cao, W. et al. Dynamics of Proliferative and Quiescent Stem Cells in Liver Homeostasis and Injury. Gastroenterology 153, 1133-1147 (2017).

51. Peranteau, W. H. et al. Correction of murine hemoglobinopathies by prenatal tolerance induction and postnatal nonmyeloablative allogeneic BM transplants. Blood 126, 1245-1254 (2015).

52. Massaro, G. et al. Fetal gene therapy for neurodegenerative disease of infants. Nature Medicine 24, 1317-1323 (2018).

53. Haeussler M, Schonig K, Eckert H, Eschstruth A, Mianne J, Renaud JB, Schneider-Maunoury S, Shkumatava A, Teboul L, Kent J, Joly JS and Concordet JP. Evaluation of off-target and on-target scoring algorithms and integration into the guide RNA selection tool CRISPOR. Genome Biol. 2016;17:148.

54. Truong DJ, Kuhner K, Kuhn R, Werfel S, Engelhardt S, Wurst W and Ortiz O. Development of an intein-mediated split-Cas9 system for gene therapy. Nucleic Acids Res. 2015;43:6450-6458.

55. Gray SJ, Foti SB, Schwartz JW, Bachaboina L, Taylor-Blake B, Coleman J, Ehlers MD, Zylka MJ, McCown TJ and Samulski RJ. Optimizing promoters for recombinant adeno-associated virus-mediated gene expression in the peripheral and central nervous system using self-complementary vectors. Hum Gene Ther. 2011;22:1143-1153.

56. Choi JH, Yu NK, Baek GC, Bakes J, Seo D, Nam HJ, Baek SH, Lim CS, Lee YS and Kaang BK. Optimization of AAV expression cassettes to improve packaging capacity and transgene expression in neurons. Mol Brain. 2014;7:17.

57. Clement K, Rees H, Canver MC, Gehrke JM, Farouni R, Hsu JY, Cole MA, Liu DR, Joung JK, Bauer DE and Pinello L. CRISPResso2 provides accurate and rapid genome editing sequence analysis. Nat Biotechnol. 2019;37:224-226.

58. Buie HR, Campbell GM, Klinck RJ, MacNeil JA and Boyd SK. Automatic segmentation of cortical and trabecular compartments based on a dual threshold technique for in vivo micro-CT bone analysis. Bone. 2007;41:505-515.

59. Bello-Arroyo E, Roque H, Marcos A, Orihuel J, Higuera-Matas A, Desco M, Caiolfa VR, Ambrosio E, Lara-Pezzi E and Gomez-Gaviro MV. MouBeAT: A New and Open Toolbox for Guided Analysis of Behavioral Tests in Mice. Front Behav Neurosci. 2018;12:201.

60. Ou L, Herzog TL, Wilmot CM and Whitley CB. Standardization of alpha-L-iduronidase enzyme assay with Michaelis-Menten kinetics. Mol Genet Metab. 2014;111:113-115.

61. Frank DB, Penkala IJ, Zepp JA, Sivakumar A, Linares-Saldana R, Zacharias WJ, Stolz KG, Pankin J, Lu M, Wang Q, Babu A, Li L, Zhou S, Morley MP, Jain R and Morrisey EE. Early lineage specification defines alveolar epithelial ontogeny in the murine lung. Proc Natl Acad Sci USA. 2019;116:4362-4371.

62. Hama H, Hioki H, Namiki K, Hoshida T, Kurokawa H, Ishidate F, Kaneko T, Akagi T, Saito T, Saido T and Miyawaki A. ScaleS: an optical clearing palette for biological imaging. Nat Neurosci. 2015;18:1518-1529.

63. Gaudelli et al., Nature Biotechnology 2020;38:892-900.

### Example II

### Adenine Base Editing to Correct the G→A (W402X) Mutation in the Canine MPS-IH model

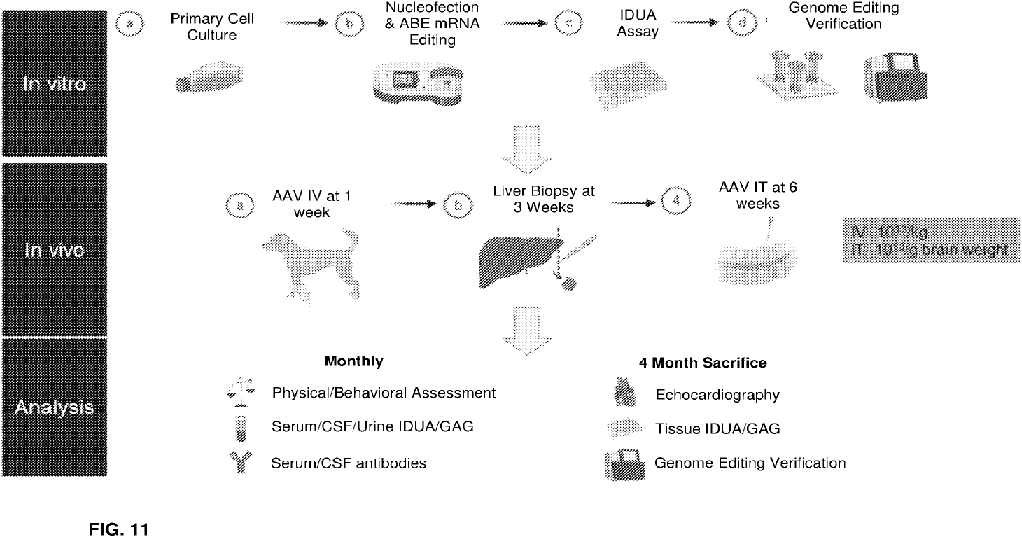

Given our findings in Example I, which demonstrated the efficacy of systemic adeno-associated virus mediated base editing to correct the manufactured W392X mutation in the murine model of MPS-IH, we also evaluated the clinical translatability of a similar approach in the spontaneous canine model of the disease. FIG. 11 depicts a schematic diagram showing the protocol for the canine study.

### Materials and Methods for the Practice of Example II

### Cell Culture

We derived diseased pulmonary fibroblasts from an affected 30-day old male MPS-I canine proband. Briefly, a freshly dissected right lung lower lobe was flushed with phosphate buffered saline (PBS) via the pulmonary artery branch. A peripheral 2 cm² of tissue was stripped of pleura, minced, and resuspended in lung digestion buffer (collagenase I, Dnase, and dispase II). After a 30-minute incubation at 37° C., the solution was processed to a single cell suspension and put through a 70 um filter. Cells were plated in coated tissue culture flasks in Dulbecco’s Modified Eagle Medium supplemented with 10% heat-inactivated fetal bovine serum and 1% penicillin-streptomycin. Primary murine pulmonary and human normal dermal fibroblasts were used as positive controls for enzyme expression.

### Genotyping and Sequencing

After achieving confluency, cells were genotyped to confirm homozygosity at the IVS1-2G>A locus. Briefly, after a PBS wash, cells were lysed with Lucigen QuickExtract DNA Extraction solution and subject to thermocycling per manufacturer instructions. On-target and genotyping PCR were conducted in 20ul reactions using Kapa HiFi polymerase supplemented with 5% DMSO and primers CaIDUA_F (5′ ctggcgctgctggccgc; SEQ ID NO:47) and CaIDUA_R (5′ ctggacggtcccgggccctg; SEQ ID NO: 48) with an annealing temperature of 66° C. Confirmation of homozygosity was determined using restriction digestion with HphI enzyme which recognizes an intact splice acceptor and results in complete cleavage of the normal canine sequence and no cleavage of the mutated sequence. Base correction was confirmed using deep sequencing performed by Genewiz using the Amplicon-EZ service. Data were analyzed using Crispresso2⁶.

### mRNA Production

Single guide RNAs (sgRNA) targeting the splice site mutation were ordered with Alt-R Crispr-Cas9 modifications from Integrated DNA Systems (IDT, Coralville, IA). The defined canine IDUA protospacers were dMPS1.1 5′ ttctgatgagggctccgcgg (SEQ ID NO: 49), dMPS1.2 5′ gcttctgatgagggctccgc (SEQ ID NO: 50), dMPS1.3 5′ ggcttctgatgagggctccg (SEQ ID NO: 51), and dMPS1.4 5′ cttctgatgagggctccgcg (SEQ ID NO: 52) and were incorporated into the standard IDT tracrRNA.

Custom ABE6.3 and ABE7.10 mRNA were designed based on plasmid sequences shared by Gaudelli et al. on Addgene and produced by Trilink Biotechnologies.

### Transfection

IVS1-2G>A fibroblasts were cultured as described above in a 6-well plate containing 1.5 mL of prewarmed cell culture media per well. Cell pellets of 5E5 cells were washed with calcium and magnesium free phosphate-buffered saline (Corning, 21-031-CVR, Corning, NY, USA) and resuspended in 100 ul nucleofector solution (Amaxa Primary Mammalian Fibroblast Nucleofector Kit VPI1002, Lonza Group AG, Basel, Switzerland). Initial transfections were performed with 10 ug CleanCap eGFP mRNA (Trilink Biotechnologies, San Diego, CA) using an Amaxa IIb nucleofector to determine optimized transfection conditions. Subsequent transfections were conducted using the program U-023 with 10 ug ABE mRNA and 10 ug sgRNA. Following transfections, prewarmed media was used to rescue and transfer transfected cells.

### IDUA Assay

At 48 h after treatment, cells were trypsinized, centrifuged, and re-suspended in triton 0.1% and then subjected to 6 freeze-thaw cycles in dry ice/methanol. α-L-iduronidase activity was induced using 4-methyl-umbelliferyl-α-L-iduronide (Glycosynth, Warrington, UK) in 0.4 M sodium formate buffer. After incubation for 30 minutes, reactions were arrested using glycine carbonate buffer and fluorescence was measured at excitation 360 nm and emission 460 nm using a fluorescent plate reader (BioTek, Winooski, VT). A standard calibration curve was generated using 4-methylumbelliferone (Sigma-Aldrich, St. Louis, MO) with arrestant buffer at a detection sensitivity of 80. Enzyme activity was normalized to cell protein content using the Pierce BCA Protein Assay Kit according to manufacturer’s instructions (Thermo Fisher Scientific, Waltham, MA).

### Results

The dog MPS-IH model also has a G→A mutation in the Idua gene analogous to humans. We have designed gRNAs amenable to correcting the canine mutation (Table 5). These guide RNAs together with an ABE including, but not limited to ABE7.10 and ABE8.17, will be delivered to MPS-IH dogs before and after birth via AAV9 and other delivery mechanisms including, but not limited to, other AAV serotypes and nanoparticle technology, to correct the disease-causing mutation and disease phenotype.

TABLE 5

ABE7.10 was effective on-target in primary canine MPS-I fibroblasts with 1:2 ABE:gRNA nucleofection

| Cas9 | gRNA | Position | Grna | PAM | ABE6.3 | ABE7.10 |
|---|---|---|---|---|---|---|
| SpCas9 | 1.1 | 6 | ttctg a tgagggctccgcgg SEQ ID NO: 49 | ggg | 15.6% | 27.5% |
| SpCas9 | 1.2 | 8 | gcttctg a tgagggctccgc SEQ ID NO: 50 | ggg | 8.6% | 0.85% |
| SpCas9 | 1.3 | 9 | ggcttctg a tgagggctccg SEQ ID NO: 51 | cgg | 12.65% | 2.5% |
| SpCas9 | 1.4 | 7 | cttctg a tgagggctccgcg SEQ ID NO: 52 | ggg | 8.25% | 3.54% |
| SpG | 4 | 5 | tctg a tgagggctccgcggg SEQ ID NO: 53 | ggaa | n/a | n/a |

TABLE 6

ABE7.10:gRNA1.1 resulted in restoration of Idua enzyme to ~6.5% of normal activity.
IDUA Activity (ng/h/mg protein)

| gRNA | Normal Dermal Fibroblast | Disease Control | ABE6.3 mRNA | ABE7.10 mRNA |
|---|---|---|---|---|
| 1.1 | 93 | 0 | 0 | 6.12 |
| 1.2 | 93 | 0 | 0 | 0.23 |
| 1.3 | 93 | 0 | 0 | 0 |
| 1.4 | 93 | 0 | 0 | 0 |

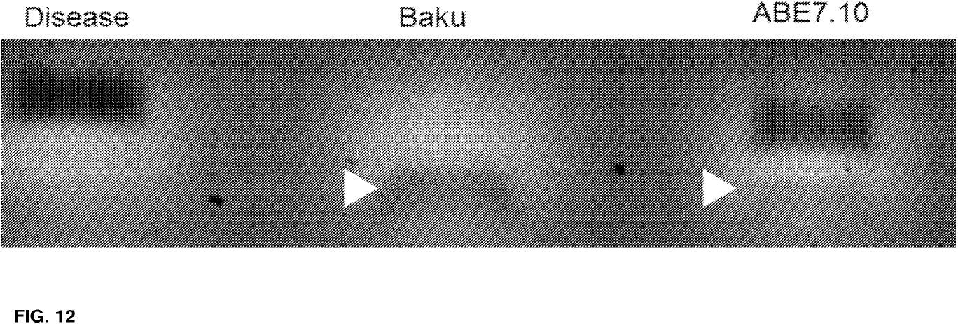

Using ABE7.10 mRNA, the gRNA sequence dMPS1.1 was identified as generating the highest on-target base editing. Notably, this sequence places the target A at an optimal position 6 in a 20-base pair protospacer 5′ of an NGG PAM (GGG). Peak editing activity of ~27.7% was seen using ABE7.10-dMPS1.1 compared to 0% in sham transfection with sgRNA alone. Notably, variable activity was seen with other guide sequences with the second highest activity seen with ABE6.3-dMPS1.3 of ~12.7%. Importantly, maximum correction was associated with IDUA enzyme activity 6.2% of normal activity compared to normal mouse and human pulmonary fibroblasts. FIG. 12 demonstrates that restriction digest provides a suitable approach for same day assessment of effective editing.

### Conclusion

Adenine base editor mediated correction of the target splice site mutation IVS1-2G>A in MPS-IH primary canine fibroblasts is associated with restoration of IDUA enzyme activity 5x greater than the 1% threshold level seen to differentiate severe human disease from a mild phenotype¹. Given our preliminary findings that suggest robust on-target correction in vivo in the murine W392X point mutation model, in primary patient-derived human fibroblasts, and in affected canine-derived fibroblasts, there is a rationale for pursuing further large animal evaluations of in vivo adenine base editing focused on safety and efficacy.

### References for Example II

1. Hopwood, J. J. & Muller, V. Biochemical discrimination of hurler and scheie syndromes. Pathology 11, 327 (1979).

2. Hinderer, C. et al. Neonatal tolerance induction enables accurate evaluation of gene therapy for MPS I in a canine model. Molecular Genetics and Metabolism 119, 124-130 (2016).

3. Simonaro, C. M. et al. Pentosan Polysulfate: Oral Versus Subcutaneous Injection in Mucopolysaccharidosis Type I Dogs. PLOS ONE 11, e0153136 (2016).

4. Miyadera, K. et al. Intrastromal Gene Therapy Prevents and Reverses Advanced Corneal Clouding in a Canine Model of Mucopolysaccharidosis I. Molecular Therapy 28, 1455-1463 (2020).

5. Kakkis, E. et al. Intrathecal enzyme replacement therapy reduces lysosomal storage in the brain and meninges of the canine model of MPS I. Molecular Genetics and Metabolism 83, 163-174 (2004).

6. Clement, K. et al. CRISPResso2 provides accurate and rapid genome editing sequence analysis. Nature Biotechnology 37, 224-226 (2019).

### Example III

### Adenine Base Editing Corrects the G→A (W402X) Mutation in Human MPS-IH Cells

A significant unmet need is present for therapies efficacious for the treatment of MPS-IH, a monogenic autosomal recessive LSD disease characterized by progressive debilitating skeletal deformities, cardiopulmonary disease, developmental delay, and mortality in the first decade of life¹. The incidence of disease in 1:100,000 and over 40% of patients carry the IDUA G→A mutation in which the loss of a tryptophan residue at position 402 results in a premature stop codon and undetectable α-L-iduronidase (IDUA) enzyme in the homozygous state². MPS-IH or Hurler syndrome portends significant morbidity and early mortality in affected children due to multi-organ disease and cardiopulmonary insufficiency. GAGs) heparan and dermatan sulfates³. In the absence of IDUA enzyme, lysosomal GAG accumulation contributes to pH alterations which result in increased lysosomal membrane permeability and release of cysteine proteases⁴. Multiple axes of cellular homeostasis-including vesicle trafficking, apoptosis regulation, autophagy, cell-cell signaling, and replication-are affected which ultimately causes multiorgan tissue pathology⁴. In the most severe form of the disease, cardiac, skeletal, and neurocognitive defects predominate and in the absence of treatment, result in death by age 10¹. Current treatments include enzyme replacement therapy (ERT) and hematopoietic stem cell transplantation (HSCT). Lifetime ERT is costly, immunogenic, and of limited efficacy⁵. In contrast, although hematopoietic stem cell transplantation (HSCT) can reduce morbidity and mortality, treatment-associated mortality is ~15% and HSCT has limited efficacy if administered after 2-3 years of life⁶. Treatment-associated morbidity, lifetime cost, and poor quality of life thus are limiting for patients with MPS-IH.

In contrast to existing therapies, genome modification approaches can precisely and curatively correct the IDUA mutation as described herein. For example, a current phase I/II clinical trial involving ex vivo modification of autologous HSCs treated with lentivirus containing an IDUA transgene and subsequent transplant has demonstrated encouraging results with improvement in serum IDUA levels in two patients^{7,8}. Gene editing studies involving CRISPR-Cas9 in multiple human MPS cell-types are also encouraging. Cas9-mediated homology-directed repair (HDR) in homozygous patient-derived fibroblasts using single-stranded oligonucleotide donor template demonstrated 4-7% allelic correction and increased enzyme activity⁹. In addition, co-delivery of Cas9/sgRNA ribonucleoprotein complex (RNP) and adeno-associated virus (AAV) serotype 6 containing a homology template targeting the CCR5 safe-harbor locus in cord-blood and adult peripheral derived HSC progenitors resulted in >75% insertions/deletions (indel) and up to 600x IDUA enzyme activity over negative controls¹⁰. Finally, transplantation of ex vivo genome edited HSCs into NOD scid gamma immunodeficient mice that also harbored the murine W392X mutation (analogous to human W402X) demonstrated normalization of peripheral GAGs and restoration of IDUA in the liver, spleen, and brain; correction of multiple skeletal phenotypes, and improvement in neuroinflammation. Nonetheless, these approaches are limited by the potential risks of Cas9 off-target mutagenesis induced by double-strand breaks (DSB), procedural risks associated with cell transplantation, narrow treatment windows, durability of genomic correction, and/or limited efficiency.

Adenine base editing has the potential to correct the most common disease-causing mutation, IDUA G→A (W402X). In this study, we assessed the efficiency of adenine base editing to correct the mutation in patient-derived fibroblasts. Selective targeting of the mutation in vitro by nucleofection or lipid nanoparticle (LNP) delivery of mRNA base editor and sgRNA was efficient, leading to correction of wild-type alleles in up to 60% of alleles. Allelic correction was associated with restoration of IDUA enzyme activity and improvements in lysosomal burden and gene expression in affected pathways in edited cells. Importantly, multiple predictive and unbiased analyses demonstrated minimal off-target DNA and RNA mutagenesis in edited cells. Finally, intravenous lipid nanoparticle (LNP) delivery of mRNA base editor in vivo led to genomic correction that was associated with liver IDUA activity above the threshold that differentiates severe and mild disease. Collectively, these findings indicated that efficiently performing therapeutic base editing can restore normal function in the humans harboring the MPS-IH mutation.

The following materials and methods are provided to facilitate the practice of Example III.

### Cell Culture

Human neonatal dermal fibroblasts from a proposita homozygous for p.Trp402* (W402X) IDUA mutation (Coriell GM00798), parent-derived dermal fibroblasts heterozygous for p.Trp402* (Coriell GM00799 or GM00800), and normal human neonatal dermal fibroblasts (NHDF) control cells (University of Pennsylvania Skin Translational Research Core) were cultured in Dulbecco’s Modified Eagle’s Medium (DMEM) supplemented with 15% FBS (Gibco) and 1% penicillin-streptomycin (Gibco). Cells were incubated at 37° C. in a humidified 5% CO2 atmosphere. Culture media was changed every 2 days and cells were passaged when 80-90% confluency was reached. W402X cells were used only until passage 12 and NHDF cells were used only until passage 22 for all experiments. For mannose-6-phosphate inhibition experiments, forty-eight hours after transfection, media in experimental wells was replaced with sterile filtered media containing 10 mM mannose-6-phosphate (Sigma).

### mRNA Production

Custom ABE6.3 and ABE7.10, and ABE8.8 mRNA were designed based on plasmid sequences shared by Gaudelli et al. on Addgene and produced by Trilink Biotechnologies. See “Phage-assisted evolution of an adenine base editor with improved Cas domain compatibility and activity.” Richter MF, Zhao KT, Eton E, Lapinaite A, Newby GA, Thuronyi BW, Wilson C, Koblan LW, Zeng J, Bauer DE, Doudna JA and Liu DR. Nat Biotechnol (2020) 10.1038/s41587-020-0453-z and “Programmable base editing of A*T to G*C in genomic DNA without DNA cleavage.” Gaudelli NM, Komor AC, Rees HA, Packer MS, Badran AH, Bryson DI, Liu DR. Nature. 2017 Nov 23;551(7681):464-471.

A single guide RNA (sgRNA) targeting the W402X mutation was ordered with Alt-R Crispr-Cas9 modifications from Integrated DNA Systems (IDT, Coralville, IA). The defined human IDUA protospacer was 5′ GCTCTAGGCCGAAGTGTCGC 3′; (SEQ ID NO: 54) and incorporated into the standard IDT tracrRNA.

### mRNA Transfection

W402X fibroblasts were cultured as described above in a 6-well plate containing 1.5 mL of prewarmed cell culture media per well. Cell pellets of 5E5 cells were washed with calcium and magnesium free phosphate-buffered saline (Corning, 21-031-CVR, Corning, NY, USA) and resuspended in 100 ul nucleofector solution (Amaxa Human Dermal Fibroblast Nucleofector Kit VPD1001, Lonza Group AG, Basel, Switzerland). Initial transfections were performed with 10 ug CleanCap eGFP mRNA (Trilink Biotechnologies, San Diego, CA) using an Amaxa IIb nucleofector to determine optimized transfection conditions. Subsequent transfections were conducted using the program U-20 with 10 ug ABE mRNA and 10 ug sgRNA. Following transfections, prewarmed media was used to rescue and transfer transfected cells. Multiple transfections were performed for each of the requisite timepoints detailed in the study.

### RNP Transfection

RNP transfections were performed in a similar fashion to mRNA transfections. First, 100 µg Cas9 protein (IDT) was precomplexed with 60 µg sgRNA at room temperature for 30 minutes prior to transfection. Subsequently, the same transfection conditions as for mRNA were employed.

### DNA Extraction and Amplicon Sequencing

On-target editing of the IDUA gene was assessed by Sanger sequencing and next-generation sequencing (NGS). Forty-eight hours after nucleofection, cells were washed with PBS, trypsinized, centrifuged and lysed using QuickExtract DNA Extraction solution (Lucigen, Middleton, WI) according to manufacturer’s instructions. Kapa HiFi polymerase was used to amplify the genomic region encompassing the W392X mutation with an annealing temperature of 66° C. PCR products were assessed using a 1% agarose gel and then purified using the Qiagen PCR Purification Kit (Qiagen, Hilden, Germany) according to the manufacturer’s recommendations. Sanger and NGS were conducted by Genewiz services (Genewiz, Plainfield, NJ). Data from Sanger was analyzed using EditR⁶⁰ and data from NGS was analyzed using CRISPResso2⁶¹. PCR primers used were Hu.IDUA-F (5′-CAATGCCTTCCTGAGCTACCAC-3′; SEQ ID NO: 55) and Hu.IDUA-R (5′-AGGTAGCGCGTGACGTAGAC-3′; SEQ ID NO: 56). Off-target amplicon sequencing was conducted in a similar fashion except that PCR was conducted using Platinum SuperFi II Hi-Fidelity DNA Polymerase (Thermo Fisher Scientific, Waltham, MA with a universal annealing temperature of 60° C.

### IDUA Enzyme Assay

At 48 h, 7 days, 14 days and 28 days after treatment, cells were trypsinized, centrifuged, and re-suspended in triton 0.1% and then subjected to 6 freeze-thaw cycles in dry ice/methanol. 200 uL of cell culture media from culture wells was obtained to assess media IDUA activity. α-L-iduronidase activity was induced using 4-methyl-umbelliferyl-α-L-iduronide (Glycosynth, Warrington, UK) in 0.4 M sodium formate buffer. After incubation for 30 minutes, reactions were arrested using glycine carbonate buffer and fluorescence was measured at excitation 360 nm and emission 460 nm using a fluorescent plate reader (BioTek, Winooski, VT). A standard calibration curve was generated using 4-methylumbelliferone (Sigma-Aldrich, St. Louis, MO) with arrestant buffer at a detection sensitivity of 80. Enzyme activity was normalized to cell or media protein content using the Pierce BCA Protein Assay Kit according to manufacturer’s instructions (Thermo Fisher Scientific, Waltham, MA).

### GAG Assay

2.5 mL of papain solution (Sigma-Aldrich) was added to 25 cm² flasks containing cells and incubated at 65° C. for 3 hours. Lysates were then then centrifuged to clarify supernatant. The Blyscan Glycosaminoglycan Assay (Biocolour, Carrickfergus, UK) was then used according to manufacturer instructions to develop a calibration curve and measure sample absorbances. Cell GAG content was normalized to lysate protein content using the Pierce BCA Protein Assay Kit.

### Protein Immunoblot

At least 24 hours after transfection, 25 cm² flasks were trypsinized and washed. Cells were lysed in RIPA buffer with protease inhibitor and subjected to 2 rounds of sonication. Clarified protein extracts were quantitated using the BCA Pierce Protein Assay Kit. Polyacrylamide gels were loaded with equal quantities of protein and run for 1-2 hours at 100 V. Membranes were equilibrated and transferred using a transfer cassette. Membranes were blocked in blocking solution overnight in 1% milk. Secondary HRP antibodies were applied for 1 hour and then incubated in ECL substrate prior to imaging. Quantification of immunoblots was conducted using ImageJ densitometry.

### Lysotracker Green DND26 Labeling

Lysosomal mass of NHDF, untreated W402X, nucleofected W402X, and M6P treated nucleofected W402X fibroblasts was evaluated 14 days after nucleofection with the fluorogenic probe LysoTracker® DND-26 (ThermoFisher, USA) according to the manufacturer’s instructions. Cell were incubated with dye (50 nM) for 15 min at 37° C. and then washed with DPBS. Live cells were then resuspended in DPBS and observed via confocal microscopy and their fluorescence intensity was analyzed by flow cytometry using a BD FACS Aria (BD Biosciences, Franklin Lakes, NJ). For each sample, at least 10,000 events were acquired.

### Digenome Sequencing

1E6 cells were lysed using lysis buffer (1 ×PBS, 0.4% NP-40, and 3 mM MgCl₂) and centrifuged at 500 g for 5 min. Supernatant was removed and nuclei pellets were mixed with nuclear lysis solution (10 mM EDTA, 0.5 mM EGTA, 0.1% Triton X-100) and centrifuged at 500 g for 5 min. Nuclei pellets were then incubation with 300 nM of Cas9 and 900 nM of sgRNA for 8 h at 37° C. in reaction buffer (100 mM NaCl, 50 mM Tris-HC1, 10 mM MgCl₂, and 100 µg/mL BSA, at pH 7.9). Cas9 protein (nM) and sgRNA (nM) were pre-incubated at RT for 10 min and mixed with 20 µg of gDNA in a reaction buffer (100 mM NaCl, 50 mM Tris-HCl, 10 mM MgCl₂, 100 µg/ml bovine serum albumin, at pH 7.9) for 8 h at 37° C. Digested gDNA was treated with RNase A (50 µg/ml) and protease K to degrade the sgRNA and Cas9 protein and repurified with a DNeasy tissue kit (Qiagen). The obtained gDNA (1 ug) was then sheared to yield fragments of 500 bp in size using a Covaris system (Life Technologies) and blunt-ended using End Repair Mix (Illumina). The fragmented DNA was then subjected to 30x whole-genome sequencing (WGS) by Illumina Genewiz. DNA cleavage scores at the DNA target sites were then calculated using the Digenome Seq web tool⁶²

### Induce-Seq

5E5 cells transfected with Cas9-sgRNA RNP and plated on poly-D-lysine coated 96 well plates. 4 hours after transfection, cells were washed and fixed with 2% paraformaldehyde. Cells were permeablized in lysis buffer and then washed 3 times in cut smart buffer (NEB B7204S). Cells were subject to blunt-end repair with the NEB quick blunting kit (E1201L and A-tailed using NEBNext® dATailing Module (NEB, E6053L. A-tailed cells were washed three times in 1x CutSmart® buffer then incubated in 1x T4 DNA Ligase Buffer (NEB, B0202S). A-tailed ends were labelled by ligation using T4 DNA ligase (NEB, M0202M) + 0.4 µM Modified P5 adapter. After washing, genomic DNA was extracted, fragmented and size selected using SPRI beads (GC biotech CNGS-00005). DNA was ligated with INDUCE-seq adapters and subjected to sequencing using an Illumina NextSeq 500 using 1x75 bp High Capacity flow cell. Reads were analyzed using the INDUCE-seq pipeline. See the world wide web at biorxiv.org/content/10..1101/202.08.25.266239v1.full.pdf.

### One-Seq

CasDesigner was used to reference the human genome hg19 for closely-matched sites to the gRNA targeting the W402X mutation. Up to 6-8 mismatches to the on-target site were included in pre selection libraries and unique sequences were subject to unique barcodes. The barcodes were flanked with appropriate indicator sequences and then DNA sequences in the ONE-seq library were synthesized on high-density oligonucleotide chips (Agilent 581 Technologies; G7238A, G7222A). Oligonucleotide libraries were made double stranded by limited cycle PCR-amplification and resuspended in solution. ABE7.10 and 8.8 mRNA and sgRNA targeting the W402X mutation were incubated with the DNA library. Cleaved products were extended, purified, end-ligated to DNA adapters, and subject to MiSeq sequencing. See the world wide web at biorxiv.org/content/10.1101/2021.04.05.438458v1.abstract.

### RNA Preparation for Whole Transcriptome RNA-seq

18 hours after transfection with ABE7.10 mRNA and sgRNA targeting the W402X mutation, cells were trypsinized, pelleted, and flash frozen in liquid nitrogen. Genewiz services were used to conduct mRNA enrichment, fragmentation and priming to generate cDNA. After end repair, 5′ phosphorylation, and dA-tailing, cDNA was adaptor ligated and underwent PCR enrichment and sequencing.

### Sequence Mapping and Variant Calling for Whole Transcriptome RNA-seq

Genewiz services were used for sequence mapping and variant calling. Sequence reads were trimmed to remove possible adapter sequences and nucleotides with poor quality using Trimmomatic v.0.36⁶³. The trimmed reads were mapped to the Homo sapiens GRCh38 reference genome available on ENSEMBL using the STAR aligner v.2.5.2b⁶⁴. Unique gene hit counts were calculated by using featureCounts from the Subread package v.1.5.2. Next, variants that fell within exon regions were tabulated and used for differential expression analysis. Gene expression analysis between sample groups was performed using DESeq2⁶⁵. The Wald test was used to generate p-values and log2 fold changes. Genes with an adjusted p-value < 0.05 and absolute log2 fold change > 1 were called as differentially expressed genes for each comparison. A gene ontology analysis was performed on the statistically significant set of genes by implementing BEAVR⁶⁶.

### Calculation of Transcriptome-level A-to-I Editing

The python package REDItools was used to quantify the percentage of A-to-I editing by sample⁶⁷. The GRCh38 reference genome was used as a reference as above. After indexing BAM files using SAMtools and referencing, REDItools was used to filter out reads with a read coverage less than 30 and an overall quality score less than 30. We then summed the total number of adenosines in the dataset. Next, the package vcftools was used to extract only A-to-G variants at loci with a corresponding A in the reference genome with a call quality greater than or equal to 30 which removed sites with less than a 99.9% probability of having a true A-to-I edit⁶⁸. To calculate the percentage of transcriptome-wide A-to-I editing in each sample, the frequency of filtered A-to-G variants was divided by the frequency of A in each dataset.

### Predicting the Effect of Transcriptome-wide A-to-I Editing

After filtering and identification of bonafide A-to-I edits as described above, the web interface for the Variant Effect Predictor⁴⁸ was employed to determine the mutation state at each locus including transcript classification and synonymous or nonsynonymous change. Next, using the advanced features of the tool, SIFT scores were generated and organized by call confidence to predict potential protein-level pathologic changes resulting from identified nonsynonymous mutations⁴⁹.

We then selected variants with a Combined Annotation Dependent Depletion (CADD) phred score ≥ 20, representing sites predicted to be the 1% most deleterious substitutions that could be exacted on the human genome⁵⁰. Given the probabilistic nature of effect prediction, we further filtered these sites to include variants that also had a high confidence SIFT score indicating deleterious variants and then rank-ordered sites by transcript frequency. We then identified sites conserved among experimental samples that did not exist in diseased control samples. Of those, we then excluded sites that were present in healthy controls to ultimately identify potential variant loci most likely resulting from deaminase activity.

### Statistics

All quantitative data was analysed in JMP 14.3 (SAS Institute, Inc., Cary, NC). Two-sided Student’s t-test was used for direct comparisons between two groups. All comparison tests were conducted with α=0.05.

### In Vitro Adenine Base Editing Precisely Corrects the W402X Mutation

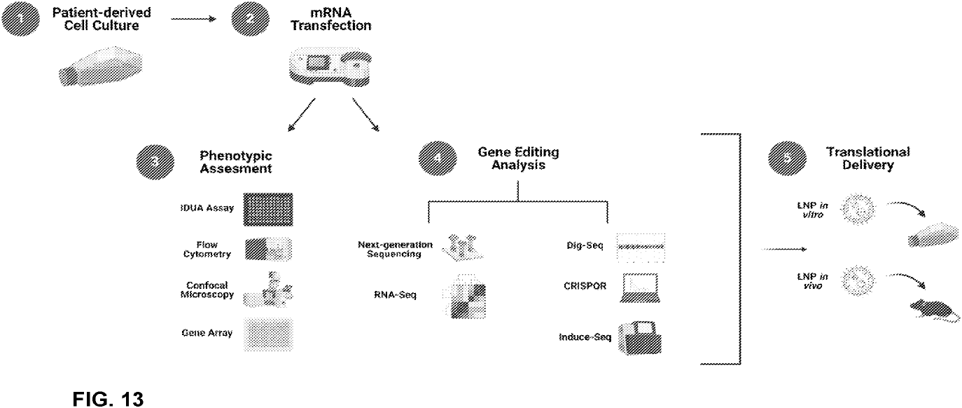

Human MPS-IH most commonly results from a point mutation W402X that leads to a premature stop codon and disruption of the IDUA gene. Due to low efficiency of plasmid transfection of primary fibroblasts, we sought to co-deliver the optimized ABE7.10 (ABEmax)^{11,13} as a nucleoside substituted mRNA with a chemically modified sgRNA targeting the W402X mutation (FIG. 13). Adenine base editors consist of a catalytically impaired Cas9 nickase, a wild-type adenosine deaminase, and version-specific deoxyadenosine deaminases that selectively convert A to G11. Accordingly, our sgRNA placed the W402X mutation at position 6 within the 20-base protospacer sequence upstream of an NGG protospacer-adjacent motif (PAM).

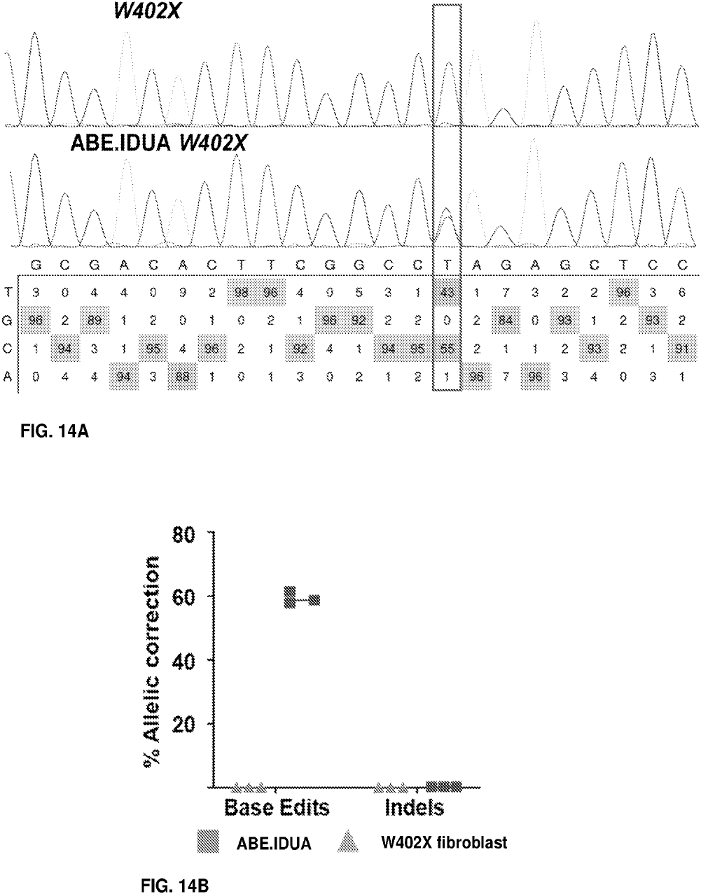

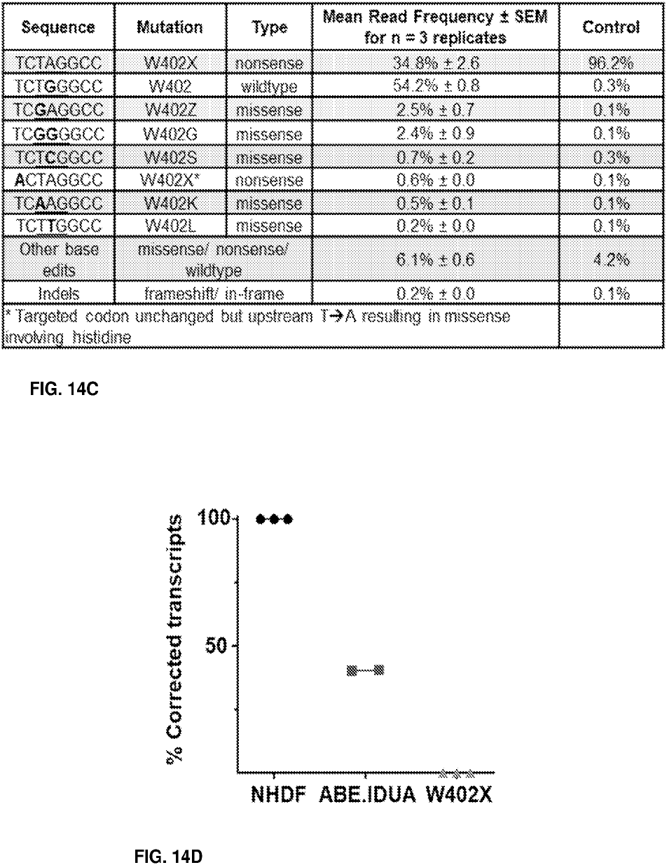

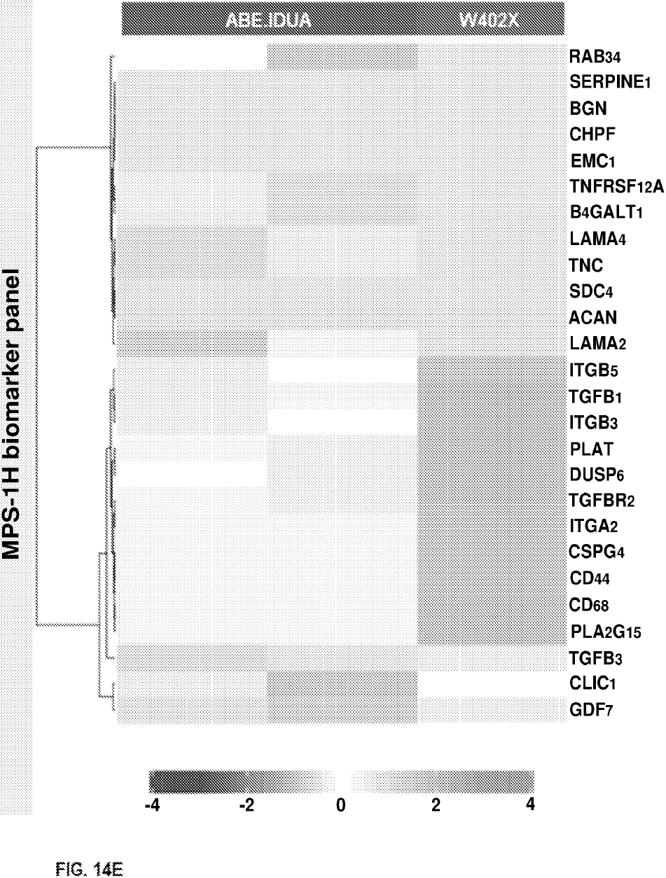

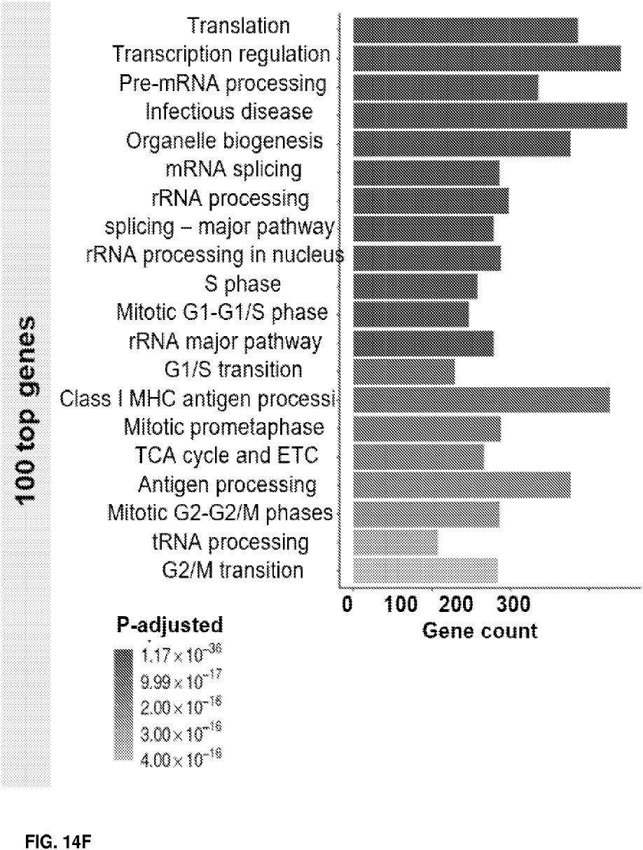

Electroporation of early passage patient-derived human MPS-IH fibroblasts (W402X) with sgRNA/ABE7.10 mRNA resulted in appropriate nucleotide correction on Sanger sequencing (FIG. 14A). On next generation sequencing (NGS) of treated cells (ABE.IDUA), we found an average ~59.3% on-target A-to-G correction, an efficiency that was stable over 28 days (FIG. 14B, Parallel transfection of MPS-I fibroblasts with GFP mRNA utilizing the same conditions suggested ~97.5% transfection efficiency. As ABE7.10 is active in the protospacer region from base ~4-711 we further quantified substitutions within an 8-base pair window around the target nucleotide (FIG. 14C). The most common observed alteration in this window was a missense G transversion with or without the correct on-target site. However, in this scenario, the resulting transcript is associated with alternate splicing and nonsense-mediated decay which renders a noncoding transcript²². In addition, other observed upstream missense and nonsense mutations do not alter the premature stop codon at the on-target site and spurious downstream mutations are not translated, suggesting that the observed in-window bystander edits are likely of minimal consequence.

In addition to substitutions, ABE can result in indel formation due to the nickase function of the Cas9, although the frequency of these events is much less than that seen with traditional Cas9 DSBs16. We therefore sought to evaluate the presence of indels in sequenced amplicons. Notably, the affected region of the human IDUA gene is highly G-C rich which contributes to significant sequencing background. However, despite background sequencing variation in control and experimental samples, there were no detectable indels in the on-target window in multiple transfections of sgRNA/ABE7.10.

To further substantiate the observed genomic correction at the W402X locus, we evaluated whole-transcriptome RNA sequencing data from cells harvested 24 hours after transfection. Reads were aligned to the hg38 genome and filtered for transcripts with read quality and coverage less than 30 which removed sites with less than a 99.9% probability of having a true adenosine to inosine (A-to-I) edit. Based on this filtered dataset, we tabulated an average ~40.7% on-target correction in transcripts encompassing the W402X locus which recapitulated the high level of correction seen at the genomic level (FIG. 14D).

Recognizing that multiple axes of cellular pathology are affected by the absence of IDUA, lysosomal dysregulation, and GAG accumulation, we also compared the overall transcriptomic profile of ABE.IDUA cells to W402X fibroblasts, healthy normal dermal human fibroblasts (NHDF) from a similarly aged child, and fibroblasts from the parents of the proband. We applied a proposed 27 gene Hurler syndrome clinical biomarker profile derived from a transcriptome-wide association study to assess overall cellular pathology²³ (FIG. 14E). In addition to this targeted assessment, we evaluated the top 100 differentially expressed genes between W402X controls, ABE.IDUA cells, NHDF controls, and parental cells (FIG. 14F). In both sets of comparisons, we observed that gene-edited cells more readily resembled healthy rather than diseased controls.

### ABE-mediated Genome Correction Results in IDUA Expression and Normalization of the Pro-Inflammatory State

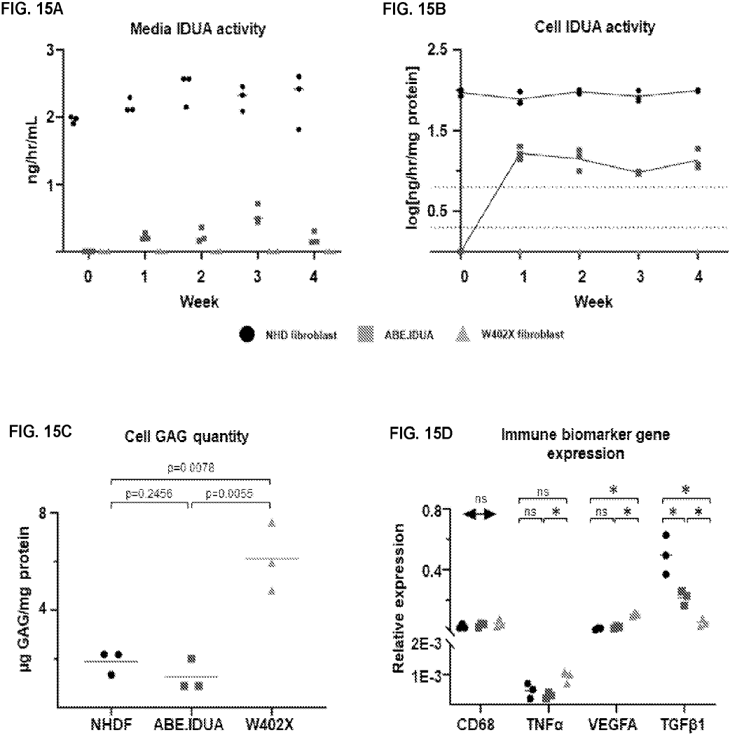

To assess whether nucleic acid correction contributed to improved cellular biochemistry, we next sampled W402X fibroblasts weekly for 28 days and compared them to ABE.IDUA (mixed population of ~59% edited and ~41% unedited cells) and NHDF. We observed detectable IDUA activity in cell lysates and media within 1 week of transfection and found that these levels achieved steady state in the same period (FIGS. 15A-15B). Although the IDUA level (~5% of normal) in ABE.IDUA did not reach that seen in NHDF, they were above the cited threshold for correction in humans from severe to mild disease^{24,25}.

Next, we evaluated whether the IDUA exposure associated with a ~59% genomic correction was sufficient to improve the overall cellular inflammatory state. We first assessed intracellular GAGs via a colorimetric assay and found that levels in ABE.IDUA were equivalent to those in NHDF and significantly reduced compared to W402X (FIG. 15C). Importantly, the accumulation of GAGs leads to lysosomal distension and dysregulation which is pro-inflammatory in nature²⁶⁻²⁹. Accordingly, we sought to assess whether the reduction in intracellular GAGs was associated with an overall improvement in the cellular immune state. To do so, we assessed 92 human inflammatory genes by quantitative reverse transcriptase polymerase chain reaction (qRT-PCR). We were particularly interested in dysregulated inflammatory genes that overlapped with the proposed clinical biomarker panel for MPS-IH²³ including CD68 which is highly expressed by macrophages and is implicated in the tissue response to GAG accumulation³⁰ and TNFα and TGFβ, part of the generalized immune response to MPS-IH31. Although CD68 did not vary between groups, TNFα and TGFβ were significantly improved in ABE.IDUA samples compared to W402X (FIG. 15D and Table 7).

TABLE 7

| Month | Group 1 | Group 2 | P-value |
|---|---|---|---|
| CD68 | W402X | NHDF | 0.279724 |
| | W402X | ABE.IDUA | 0.523435 |
| | NHDF | ABE.IDUA | 0.452344 |
| TNFα | W402X | NHDF | 0.068587 |
| | W402X | ABE.IDUA | 0.008971 |
| | NHDF | ABE.IDUA | 0.34649 |
| VEGFA | W402X | NHDF | 0.00432 |
| | W402X | ABE.IDUA | 0.00819 |
| | NHDF | ABE.IDUA | 0.0103258 |
| TGFβ1 | W402X | NHDF | 0.004146 |
| | W402X | ABE.IDUA | 0.005814 |
| | NHDF | ABE.IDUA | 0.023704 |

In addition to these biomarkers, an assessment of the broader panel of immune markers revealed 16 genes that were different between disease and healthy controls, of which VEGFA, a key mediator of the oxidative response to lysosomal dysregulation^{32,33}, was found to be significantly reduced in ABE.IDUA compared to W402X. Thus, restoration of enzyme in treated cells was associated with improved global cellular phenotype.

### IDUA Expression Is Associated With Improved Lysosomal Burden In ABE.IDUA

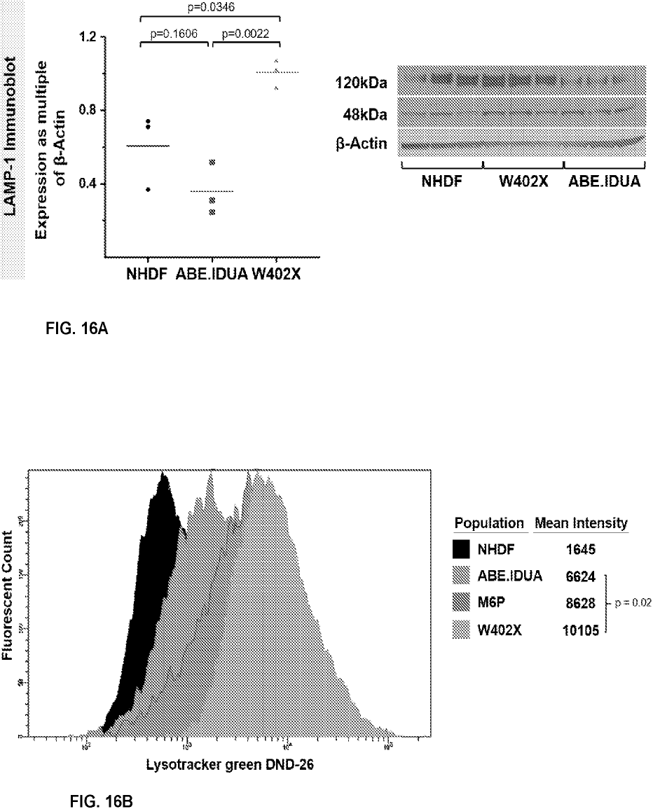

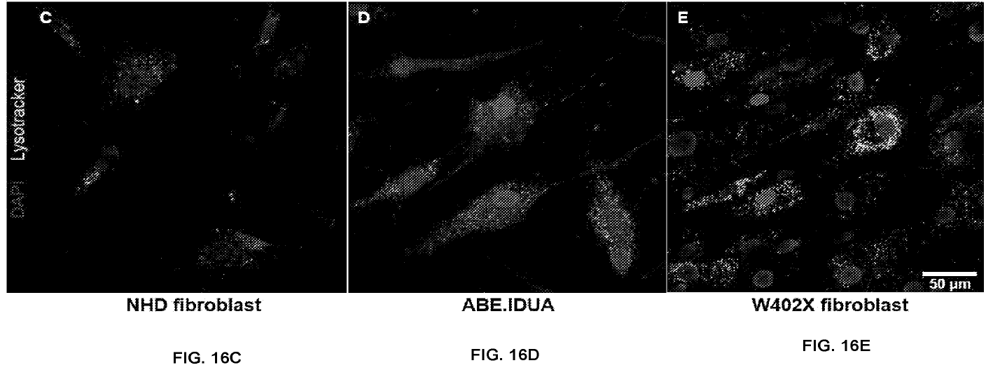

We next sought to assess whether restoration of active IDUA enzyme would result in an improvement in the specific lysosomal phenotype. In MPS-IH, the accumulation of GAGs leads to both an increase in size and number of lysosomes and resulting dysfunction leads to disruption of cellular homeostasis³⁴. We assessed LAMP-1, a marker of late endosomes and lysosomes, to assess the burden of lysosomes in cells³⁵. On immunoblot analysis, ABE.IDUA had normalized levels of normalized LAMP-1 compared to W402X (FIG. 16A).

To further assess lysosomal mass, we live-stained ABE.IDUA, W402X, and NHDF fibroblasts with lysotracker green DND-26, a fluorescent marker of acidic compartments9. On fluorescent activated cell sorting (FACS) to assess the overall fluorescent signature in cells, ABE.IDUA again demonstrated statistically significantly reduced lysosomes compared to diseased cells (FIG. 16B). Notably, a common gene therapy strategy to address disease related to secretory enzymes including MPS-IH is to induce supratherapeutic expression in one organ such as the liver to support delivery of functional enzyme to other organs^{36,37}. This strategy is supported by media transfer experiments that suggest IDUA-containing media from one group of normal cells improves pathology in recipient diseased MPS-IH cells³⁸. Although such experiments approximate the possibility of distant IDUA transfer in vivo as donor and recipient cells live in separate spaces, they do not address the potential local effects of corrected cells on surrounding diseased cells. We initially hypothesized that FACS quantification of the lysosomal signature in our mixed population of edited cells would generate a bimodal distribution -corrected cells with lower lysosomal mass and uncorrected cells with higher lysosomal mass. However, in contrast, we found that the entire mixed population of transfected cells had reduced mass. To confirm the causal hypothesis linking enzyme secretion and cross-correction, we grew transfected cells in media containing mannose-6-phosphate (M6P), a competitive inhibitor of the M6P receptor responsible for the bulk of IDUA uptake³⁹. When compared to transfected cells grown in normal media, we observed that M6P inhibited cells demonstrated a significant increase in lysosomal mass, though a distinct bimodal distribution was not seen (FIG. 16B). Taken together, these findings suggest that enzyme secretion from edited cells have a substantial cross-corrective effect on surrounding cells.

Finally, these findings were recapitulated on confocal microscopy in which ABE.IDUA cells demonstrated a reduced staining pattern compared to W402X controls after 14 days that approximated the appearance of staining in NHDFs (FIGS. 16C-16E). Taken together, these findings suggest that restoration of enzyme activity was associated with a reduction in lysosomal mass, an important inciting event for MPS-IH tissue pathology.

### mRNA Delivery Of ABE.IDUA Results In Minimal DNA Off-Target Effects

Despite the high fidelity reported with respect to ABE, off target base editing may still arise from gRNA-dependent or independent events. Specifically, substitutions or indel formation may result due to the nickase function of the Cas9, although with lower frequency than that seen with traditional non-nickase Cas9. Guide RNA-independent off-target DNA editing may arise from activity of the deaminase domain in a Cas9-independent manner.

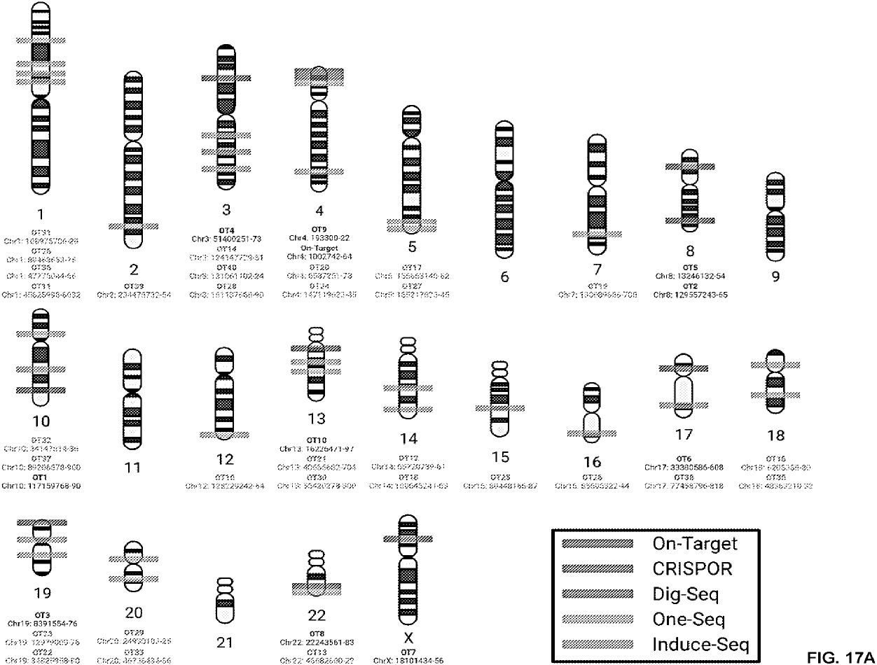

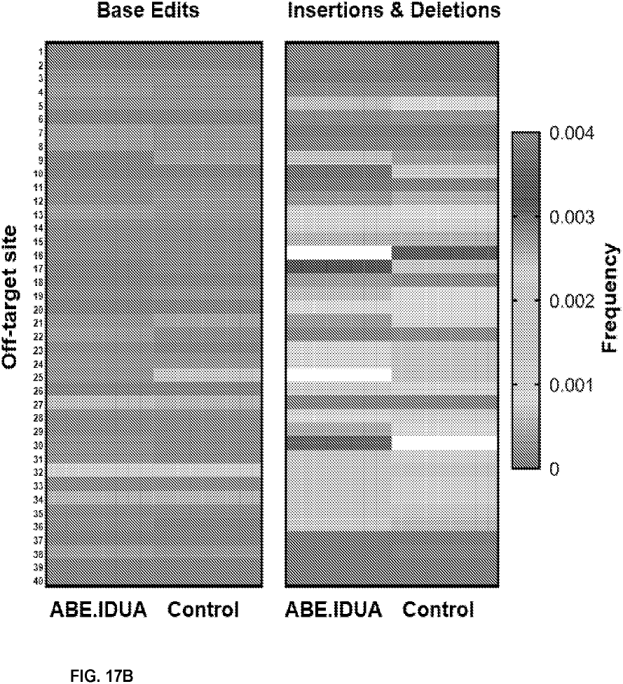

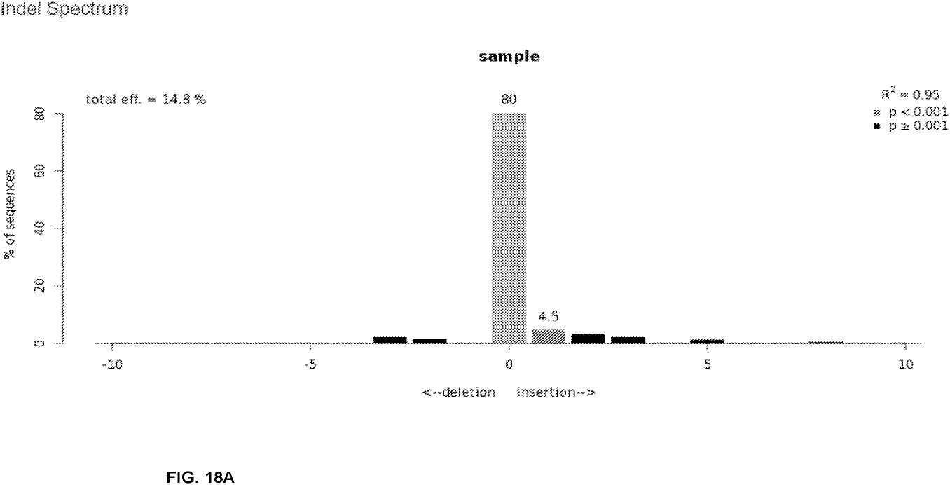

To evaluate DNA off-target mutagenesis associated with our sgRNA, we sequenced the top 10 off-target sites predicted by CRISPOR. One site was found to have significant sequencing background in control samples and was excluded. The remaining nine sites demonstrated no detectable off-target substitutions or indels above background suggesting a favourable predictive off-target profile (FIG. 17 and Table 8).

TABLE 8

CRISPOR Off-Target Sites

| Site | Location | Off-target Homologous Guide |
|---|---|---|
| Idua | Chr4 Exon: Idua | GCTCTAGGCCGAAGTGTCGC AGG (SEQ ID NO: 1) |
| OT1 | Chr10 Exon: MIR3663HG | GCTCTGGGCTGGGGTGTCGC TGG (SEQ ID NO: 57) |
| OT2 | Chr8 Intergenic: MIR3686-RP11-419K12.1 | GCTCTAGGCTGAAGTGCTTC TGG (SEQ ID NO: 58) |
| OT3 | Chr19 Intergenic: RAB11B-AS1/RAB11B-RAB11B | CCACTAGGCCAAAGTGTAGC TGG (SEQ ID NO: 59) |
| OT4 | Chr3 Intergenic: RBM15B/VPRBP-VPRBP | GCTCCAGGAGGAAGTGTCAC AGG (SEQ ID NO: 60) |
| OT5 | Chr8 Intron: DLC1 | GTTCTAGGTGGAAGTGTTGC TGG (SEQ ID NO: 61) |
| OT6 | Chr17 Intergenic: ASIC2-AA06 | GGTCTAGGCCAAGCTGTCGC TGG (SEQ ID NO: 62) |
| OT7 | ChrX Intron: LINC01456 | GCTCTAGGCAGAAGAGTTGA CGG (SEQ ID NO: 63) |
| OT8 | Chr22 Intergenic: IGLV10-54-VPREB1 | GATCAAGGCTGAAGTGTCCC TGG (SEQ ID NO: 64) |
| OT9 | Chr4 Intergenic: RP11-1398P2.1-FAM53A | GCTCCAGGCCCAAATGTCCC TGG (SEQ ID NO: 65) |
| OT10 | Chr13 Intergenic: ENSG279924 | GCTCTAGGCCGAAG—TGT-CGC AGG (SEQ ID NO: 66) |
| OT11 | Chr1 Intergenic: CCDC17-GPBP1L1 | GCTCTAGGCCGAAGAGGCGG GAG (SEQ ID NO: 67) |
| OT12 | Chr14 Intron: FUT8 | ACTATAGGCTGAAGTGTCCT GGG (SEQ ID NO: 68) |
| OT13 | Chr22 Exon: GRAMD4 | GCTCTAGAACGAAGTTCACA GGT (SEQ ID NO: 69) |
| OT14 | Chr3 Intron: KALRN | TCACTAGGCCAAAGTGTATC TGG (SEQ ID NO: 70) |
| OT15 | Chr18 Intron: L3MBTL4 | TCTCTAGGTCAGAAGTGTCA CTG (SEQ ID NO: 71) |
| OT16 | Chr12 Intergenic: LINC02368-LOC107987176 | GCTTTAGGCAGAAGTGTCAA GGA (SEQ ID NO: 72) |
| OT17 | Chr5 Intergenic: PPIGP1-SGCD | GCTCTAAGGCCAAAGTGTGC TTG (SEQ ID NO: 73) |
| OT18 | Chr14 Intergenic: BEGAIN-LINC00523 | TCACTAGGCAAAGTGTCACA GGC (SEQ ID NO: 74) |
| OT19 | Chr7 Intron: KLHDC10 | GCTCCAGGCAAAGTGTCTAA GGG (SEQ ID NO: 75) |
| OT20 | Chr4 Intron: MAN2B2 | GCTCTATGCCGAAGTGTTCG GAA (SEQ ID NO: 76) |
| OT21 | Chr13 Intron: FOXO1 | GCTCAGGCCGGAGTGTCGTG GCA (SEQ ID NO: 77) |
| OT22 | Chr19 Intergenic: LINC01801 | AGACTAGGCTGAAGTGTCCC AGG (SEQ ID NO: 78) |
| OT23 | Chr19 Intergenic: DAND5-NFIX | CATCTAGGGGAAGTGTCACT GGG (SEQ ID NO: 79) |
| OT24 | Chr15 Intron: ARNT2 | ACTCTAGGTCAAAGTGTCAG CTG (SEQ ID NO: 80) |
| OT25 | Chr16 Intron: GSE1 | ACTCTAGGCCCAATGTCCCA GGG (SEQ ID NO: 81) |
| OT26 | Chr1 Unchar: LOC105378839 | TCTCTAGGACAAAGTTGTCT CAG (SEQ ID NO: 82) |
| OT27 | Chr5 Intergenic: LOC105377702-LINC01938 | GCTCTATTAGAAGTGTAGCA GGA (SEQ ID NO: 83) |
| OT28 | Chr3 Intron: SOX2-OT | TCTCTAGGCCGAAGTCTTAG GGA (SEQ ID NO: 84) |
| OT29 | Chr20 Unchar: LOC105372578 | TCTCTAGGCTGAAGTGCCCT GGG (SEQ ID NO: 85) |
| OT30 | Chr13 Intergenic: MIR5007-HNF4GP1 | TCTCTAAGGAAGTGTCACAG GAG (SEQ ID NO: 86) |
| OT31 | Chr1 Intron: WDR47 | GCTCTAGACCAAAGTGGTCT AGG (SEQ ID NO: 87) |
| OT32 | Chr10 Intron: PARD3 | GCTCTAGGGCCAAAGGTCAC AGG (SEQ ID NO: 88) |
| OT33 | Chr20 Intergenic: SLC2A10-RN7SKP33 | GCTCTAAGCAGTAGTGTGCT GGT (SEQ ID NO: 89) |
| OT34 | Chr4 Intergenic: LOC105377476-MIR584G | TCTCTAGGCAGAAGTGATGC TGG (SEQ ID NO: 90) |
| OT35 | Chr18 Intron: ZBTB7C | GCTCTAGGCAGGAGGGTCCC TCC (SEQ ID NO: 91) |
| OT36 | Chr1 Intron: TRABD2B | GCTCTAAGGAAGTGTGGCAG GTG (SEQ ID NO: 92) |
| OT37 | Chr10 Exon: CHT25H | ACTCCATGTCGAAGAGTAGC AGG (SEQ ID NO: 93) |
| OT38 | Chr17 Intron: Septin9 | GCTCTAAGATGGAGTGTCCC AGG (SEQ ID NO: 94) |
| OT39 | Chr2 Intergenic: HSPE1P9-ARL4C | ACTCTAGGAAGAAGTGACTC AGG (SEQ ID NO: 95) |
| OT40 | Chr3 Intron: NEC11 | TCTCTAGGCCTAAGTGTGGA AAG (SEQ ID NO: 96) |

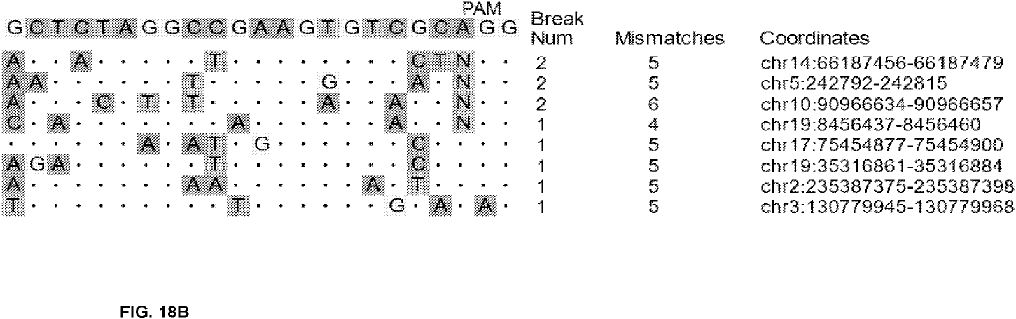

With the knowledge that prediction of potential off-target sites can be biased by guide homology, we also sought to evaluate off-target effects using Dig-Seq, an in vitro strategy that uses whole genome sequencing to assess the presence of aligned DSBs in chromatin-intact DNA40 (FIG. 17, FIG. 18, and Table 8). After in vitro Cas9/sgRNA RNP cleavage targeting the W402X mutation, 8 sites of cleavage with scores above 2.5 were identified including the on target site⁴¹. Two of the seven candidate off-target sites were within one base pair of each other and were considered the same for further analysis. Notably, semi-global alignment scores for all sites except the on-target site were less than 55, a previous cutoff used to determine likely binding sites for Cas9 cleavage⁴². In addition, five of six sites were in repeat regions and as a result we were unable to generate unique amplicons amenable to targeted sequencing. Finally, evaluation of the editing window in the remaining candidate site-found within an intergenic region and with the highest alignment (41) and cleavage (82.5) scores of the candidate sites-revealed no A to G substitutions and indel rates of 0.37% (experimental) and 0.13% (control) in targeted amplicon sequencing of ABE.IDUA or W402X fibroblasts.

Due to possible amplification bias associated with Dig-Seq, we also evaluated off-target DNA mutagenesis using INDUCE-Seq⁴³, a technique that attempts to overcome PCR-amplification bias which can reduce the signal-to-noise ratio in genomic regions with low signal such as in areas of rare DSBs. The technique involves using NGS flow cell enrichment using modified sequencing primers to directly bind blunt ends in nuclease-treated cells. 4 hours after treatment with Cas9/sgRNA nucleofection of W402X fibroblasts, cells were fixed and permeabilized for primer binding. After flow cell enrichment 5 unique sites were identified that had aligned breaks suggestive of Cas9 activity. Notably, all sites had at least 4 mismatches with respect to guide homology. Of 5 sites identified via INDUCE-Seq, we were able to generate sequence compatible amplicons for 4, all of which demonstrated no evidence of base edits or indels.

To further assess the potential translatable off-target profile of ABE.IDUA, we finally applied One-Seq, a novel strategy that minimizes nomination bias common to other in vitro whole genome based off-targeting assessments^{19,44}. In particular, nomination bias refers to the ability to detect unwanted mutagenesis at specific sites due to the burden of excess unrelated DNA sequences in the treatment pool which leads to high sequencing background and unwanted excess sequencing reads that diminish the detection threshold for true off-target events. The technique also addresses the limitation of individual sequence variants that may bias understanding of off-targeting at the population level and uses a custom barcoded DNA library harboring all DNA sites in the reference genome with a certain number of guide mismatches. Using One-Seq we assessed sites by employing a detection 0.001 which suggests that if on-target editing was 100% the maximum off-target activity would be 0.1%, below the detection threshold of NGS. We then assessed the top 32 sites with scores ranging from 0.0007 to 0.0016 by using targeted amplicon sequencing to compare ABE.IDUA and W402X samples. Notably, the highest predicted off-target site had a score of 0.0016 which suggests that if on-target editing was 100%, off target activity would be 0.16% at the site. Of 32 sites, we were able to generate sequence compatible amplicons for 26, all of which demonstrated no evidence of base edits or indels.

Overall, the absence of off-target changes at 40 identified sites of potential mutagenesis using 4 nomination techniques and the finding that remaining candidate cleavage sites determined by unbiased evaluation featured poor guide alignment and repetitive sequences suggests that the selected sgRNA was extremely precise with respect to genome-wide off-target DNA activity, suggesting potential clinical translatable compatibility.

### mRNA Delivery Of ABE.IDUA Does Not Result in Substantial Transcriptome-Wide RNA A-To-I Editing

While assessing DNA off-target effects due to adenine base editing is of critical importance, understanding potential off-target effects on the RNA transcriptome may also have clinical implications. Although to-date the transcriptomic effects of base editors have not been directly linked to pathology, dysregulation of endogenous adenine deaminases acting on RNA (ADAR) enzymes in humans can modulate immune activation and checkpoint control in tumor models^{45,46}. Notably, the wild-type tRNA-specific adenosine deaminase component of the ABE system may catalyze deamination of cellular RNA, resulting in off-target RNA edits⁴⁷.

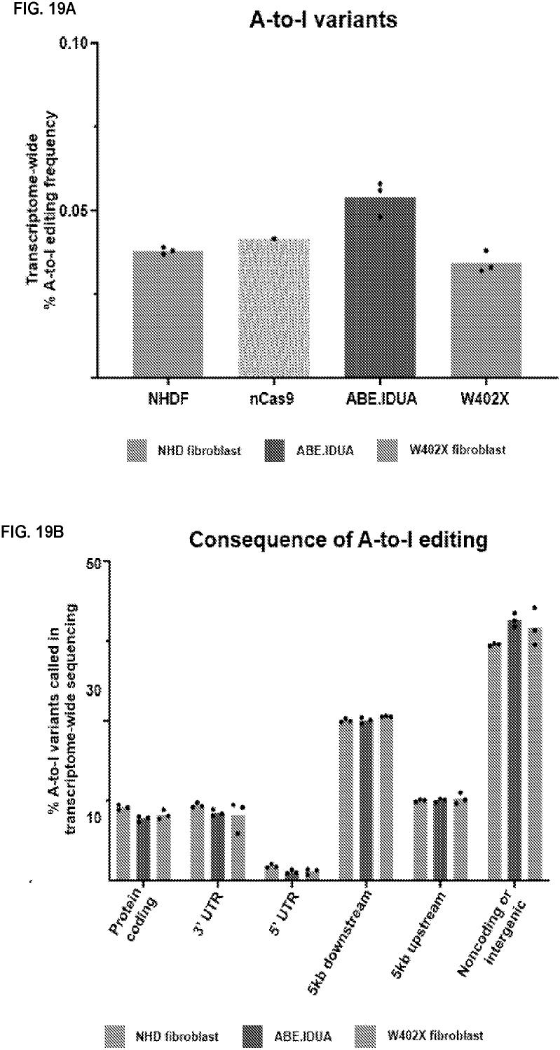

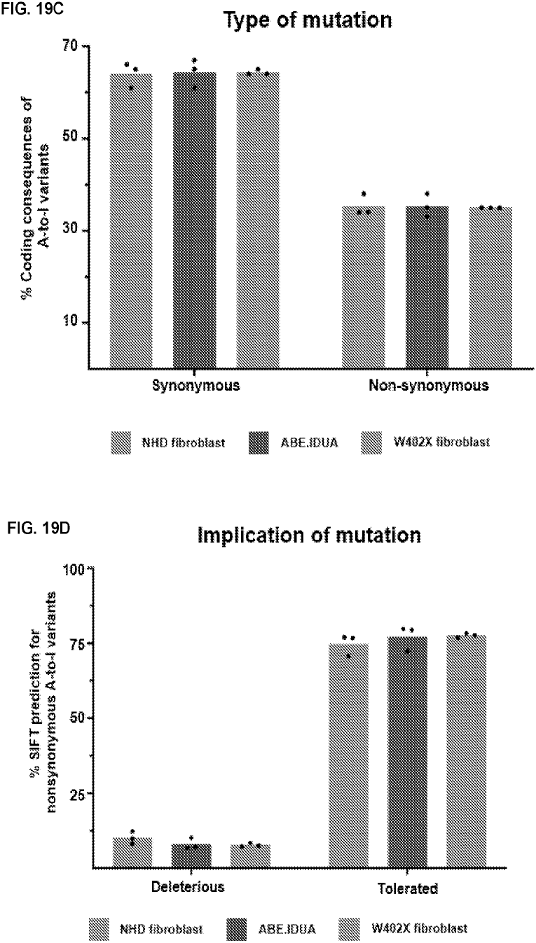

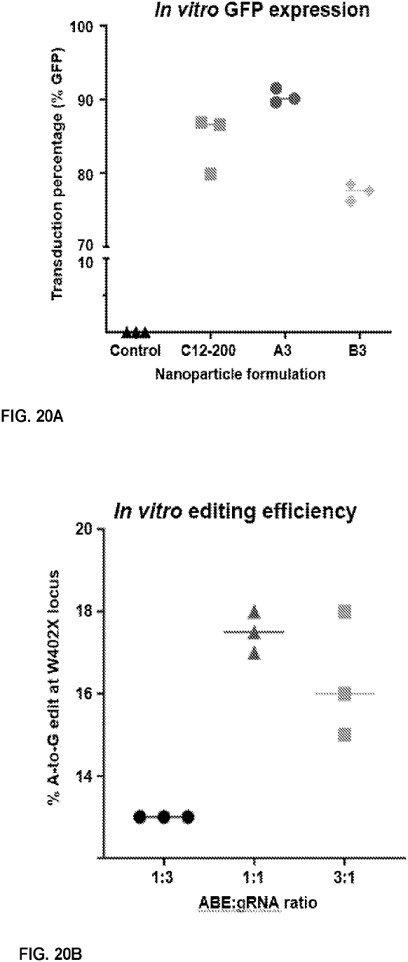

To investigate off-target RNA mutagenesis, we performed whole transcriptome RNA sequencing (RNA-Seq) to identify transcriptome-wide A-to-I editing frequencies associated with ABEmax in the context of the selected sgRNA targeting the W402X mutation. A-to-I RNA editing was seen at a ~59% higher frequency in transcripts from ABE.IDUA (0.054%) samples compared to W402X (0.034%), NHDF (0.038%), update with ncas9, suggesting low-level transcriptome-wide RNA editing consistent with prior studies^{16,19,47} (FIG. 19A).

To further assess the implications of potential edits, we then used the Ensembl Variant Effect Predictor48 to determine location of edits in all sequenced mRNA transcripts with an A-to-I edit and found comparable rates of A-to-I edits in protein coding regions across experimental and control samples (FIG. 19B). Of these, in all groups, 35% were non-synonymous mutations (FIG. 19C). Next, using the tool Sorting Intolerant From Tolerant (SIFT)49, which attempts to determine the effects of coding changes on protein function, we found that 8.0% of nonsynonymous coding A-to-I mutations in ABE.IDUA transcripts would be predicted to have a deleterious impact on protein function, no different than in control cells (FIG. 19D).

Finally, we sought to compare predicted deleterious loci in ABE.IDUA transcripts to W402X transcripts to determine locations that were unique to the experimental condition. Given the potential statistical bias in individual protein prediction models, we cross-referenced edited sites in ABE.IDUA transcripts in two prediction models. Variants with a Combined Annotation Dependent Depletion (CADD) score ≥ 20, representing those predicted to be the 1% most deleterious substitutions that could be exacted on the human genome⁵⁰, were cross-referenced with high-confidence SIFT predictions and resulting sites were rank-ordered to identify probable deleterious variants. We then attempted to identify sites conserved among experimental samples that did not exist in W402X or NHDF control samples which would represent variants most likely to be associated with adenine deaminase function and with potential deleterious protein function. Ultimately, we identified no high-risk genes that were uniquely modified in the ABE treated group compared to either control group. Moreover, our findings suggest that although low-level transcriptome-wide editing may occur in ABE.IDUA, the vast majority of identified sites were of benign or unknown consequence. Nonetheless, further assessment of mutations in regulatory regions, particularly with respect to checkpoint blockade and immune regulation will be critical in the context of more evolved ABEs optimized for minimal RNA editing prior to clinical translation.

### LNP Delivery of ABE.IDUA mRNA Is Viable in Vitro and in Vivo

Although in vivo delivery of ABE.IDUA would ideally efficiently target all affected organs including the brain, heart, liver, muscle, and bone, an alternate strategy is to achieve a sufficient steady state level of IDUA expression that facilitates organ cross-correction.

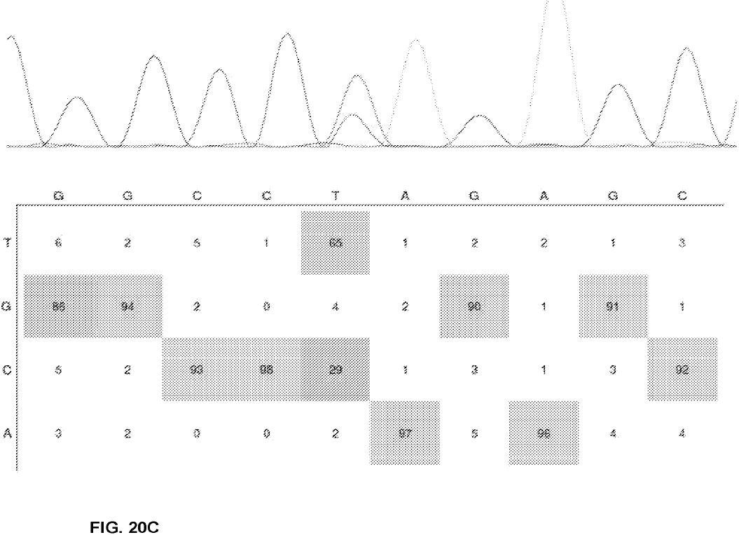

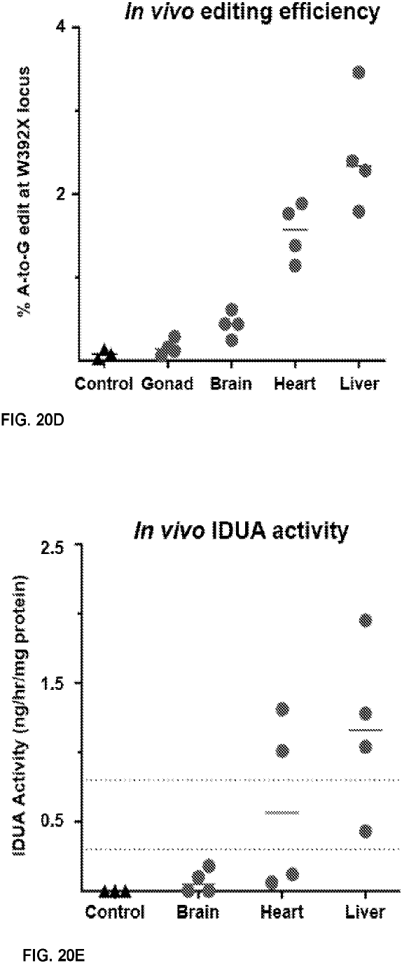

In this pursuit, we previously screened a panel of liver-targeting ionizable LNPs to optimize delivery to the fetal mouse liver¹⁷ and identified two top performers, A3 and B3. Given that the ultimate editing target would be the human hepatocyte, we first compared the efficiency of A3, B3, and a well-known publicly available LNP, C12-200 with respect to their delivery of eGFP mRNA to the HUH7 human liver cell line and found that A3 was the best performer with 90.4% transduction at 24 hours (FIG. 20A). We then assessed whether A3 could efficiently package ABE.IDUA and effect genomic change by modulating the total ABE:gRNA ratio employed in manufacturing. We found that a 1:1 ratio was as measured by Sanger sequencing 24 hours after delivery of 500 ng of mRNA was optimal (FIG. 20B). Finally, with LNP A3 delivery of 2 ug ABE.IDUA mRNA we observed up to 29% allelic correction.

Based on these results, we then sought to evaluate if LNP A3 would be functional in vivo. As such, we packaged A3 with ABE mRNA and sgRNA (A3.Idua) targeting the murine W392X mutation which is known to efficiently correct the murine MPS-1H mutation²¹. We then injected 6 10-week old homozygous W392X mice with 1.5 mg/kg of A3.Idua. At one-week and one-month post injection we observed no editing above background in the livers of injected mice. As such we adopted the dosing strategy employed by Song et al. and injected four 10-week old W392X mice with 1.5 mg/kg of A3.Idua every second day for a total of 3 doses. On NGS one week after injection, we observed editing in the heart (~1.5%) and in the liver (~2.5%). These levels were correlated with restoration of tissue IDUA activity in the heart (~0.6 log[ng/mg/hr]) and in the liver (~1.2 log[ng/mg/hr]), the latter of which was above the stated transition level differentiating severe from mild disease.

Collectively, these data demonstrate the possibility of non-viral delivery of ABE to target the adult mouse liver and a dose-dependent genotype-biochemical phenotype correlation that indicates the need to continue to optimize LNP processing and mRNA stability to maximize the effect of clinical ABE delivery.

### Discussion

CRISPR-mediated base editing offers the potential to selectively correct disease-causing mutations. In contrast to current investigational therapies including traditional Cas9-based approaches, base editing is advantageous as it is efficient and precise, does not require DSBs or the introduction of a homology template, and can be applied in vivo without requisite bone marrow harvest, ex vivo expansion, or myeloablative conditioning^{5,7,11}. In this study, we evaluated the potential of an adenine base editor to correct the W402X mutation in primary patient-derived human fibroblasts.

Precision editing with preservation of genomic integrity is of essential concern for clinical translation of base editing strategies. In our analyses, the average G→A mutation correction efficiency was ~59.3% across multiple transfections, and we observed few bystander mutations within the editing window, a finding that likely benefited from the presence of a single edit amenable nucleotide in the base editing window. Those bystander edits that were observed were missense or nonsense but are likely of marginal consequence as the default disease state in MPS-IH is production of the truncated IDUA enzyme. In addition, the rates of bystander edits observed in our amplicon are likely overestimated as multiple mutations appear to be unlikely transversions and are likely due to sequencing variation and amplification errors related to the high G-C content of the IDUA gene. Nonetheless, bystander edits are of important consideration in other diseases in which additional mutations proximal to the target site could be deleterious to a necessary hypomorph state. Therefore, continued efforts to evolve base editors to improve the editing window will be critical for optimizing on-target specificity.

Of consequence, allelic correction was associated with long-term restoration of IDUA enzyme activity and improvement in cellular lysosomal mass. IDUA was detected in cell lysates and media by a fluorescent substrate method and was found to be ~5% of normal in mixed populations of corrected and uncorrected cells. Surprisingly, the benefits of allelic correction were not limited to edited cells. Rather, we observed cross-correction of cellular pathology in unedited cells due to the secretion of IDUA from edited cells. Namely, we were unable to define two distinct populations of cells (large and small lysosomal mass) by flow cytometry in a mixed population of corrected and uncorrected cells. Instead, we found a population reduction in lysosomal signature that was competitively inhibited by the addition of mannose-6-phosphate, thus substantiating a causal relationship. In addition, we saw that gene editing rates were stable in the mixed population over 4 weeks. Collectively, these findings suggest no proliferative advantage for edited MPS-IH cells in the mixed population, likely because the overall burden of pathology is reduced due to cross-correction. Notably, in the mouse model of MPS-IH that recapitulates the W402X mutation described in Example I, we found that liver editing levels at 1 month, 4 months, and 6 months were not different in mice that had undergone in utero base editing, suggesting that the absence of proliferative advantage may also exist in vivo²¹. This finding that may also occur in other secretory pathologies, is in contrast to other diseases such as fumarylacetoacetate hydrolase deficiency in which edited cells have distinctly improved survival and a clonal advantage which facilitates propagation and increased observed editing over time⁵¹. If there is no selective advantage for edited cells in MPS-I, then the observed correction efficiency would be stable over time as edited and nonedited cells would expand at the same rate. Therefore, optimizing initial correction in secretory diseases like MPS-I will be of relevance to durable and efficacious disease correction. In this study, we use an mRNA base editor to achieve high level editing. Nonetheless, previous work suggests a minimum 0.8-1% enzyme expression level is seen in patients with the mild form of MPS-I (Scheie, MPS-IS) which results in normalized lifespan and abrogated neurologic deficits^{24,25,52,53}. Therefore, it the level of observed correction in our experiments should suffice for therapy and substantial clinical improvement in MPS-IH.

Restoration of IDUA enzyme in ABE.IDUA cells was also associated with improved cellular function. First, we observed normalized GAG levels in treated cells compared to disease control cells. This was associated with multiple reduced markers of lysosomal burden and an improved pro-inflammatory state. Finally, these findings were associated with globally improved appearance of the transcriptome in edited cells that ultimately approximated that of healthy control cells. An evaluation of key dysregulated pathways in MPS-IH demonstrated improved cellular gene expression with respect to lysosome dysregulation, autophagy, extracellular matrix proteins, and GAG processing which suggests a robust genotype-phenotype correlation in treated cells.

Notably, mRNA based editing is transient in nature compared to DNA based approaches such as lentiviral delivery and may have improved DNA off-targeting activity⁵⁴. With regard to distant off-target activity, we observed no meaningful Cas9 or deaminase activity in DNA at sites predicted by guide homology. Importantly, predictive off-targeting algorithms do not readily account for the stochastic nature of Cas9 interactions with the genome. Therefore, we sought to utilize Dig-Seq and Induce-seq, two assessments that attempt to reduce homology bias and PCR amplification bias respectively, in determining off-target activity^{40,43}. In so doing, we expected to identify off-target sites that would not be predicted by guide homology. Notably, previous work suggests that in the context of mismatched sgRNAs, off-target effects are hindered by a closed chromatin structure by up to 1000-fold, implying that the chromatin state contributes significantly to Cas9 specificity⁵⁵⁻⁵⁸. As the ultimate clinical translation of gene editing will involve manipulation of DNA in a predominantly chromatin intact state unless cells are cycling (e.g. with in utero treatment or in replicative organs), we elected to these in vitro methods as they account for this native structure^{40,43}. In our analysis, conducted after Cas9/sgRNA RNP cleavage of either nuclei pellets in which chromatin structure is retained or in fibroblasts, all identified candidate cleavage sites demonstrated poor guide homology and 5/6 candidate sites were in areas of repeat sequences. However, one limitation to our approach was the use of Cas9 protein to formulate the digestion RNP-in which the Cas9 induces a DSB rather than performs a nickase function-which likely overestimates actual cleavage events compared to ABE on WGS. To account for the bias of in vitro digestion, excess noise-generating DNA sequences in amplification-based approaches, and to specifically account for ABE rather than Cas9 editing, we used One-Seq to assess DNA off-targeting⁴⁴. Critically, the highest potential off-target site identified using an oligonucleotide library was seen to have low predicted off-target effects and in targeted amplicon sequencing, no off-target activity was detected above sequencing background at any site with predicted editing above sequencing background. Finally, as previously described^{16,19,47}, transcriptome-wide RNA sequencing in ABE.IDUA demonstrated low-level A-to-I editing with no unique sites identified in treated samples predicted to be deleterious in nature. Optimized next-generation adenine base editors and delivery vectors such as those capable of delivery ribonucleoproteins can abrogate the potential implications of off-target RNA editing⁴⁷. Here, we observed efficient and precise correct of the common W402X mutation after nucleofection of sgRNA/ABE7.10 mRNA with minimal DNA and RNA off-target effects throughout the human genome transcriptome.

Despite observed restoration of the cellular phenotype, efforts to further augment IDUA enzyme expression may be of value for ensuring adequate organism-wide correction. LNP data Indeed, in vivo correction efficiency is dependent on native editor efficiency, vector, delivery route/target, and dose^{16,29-31}. For example, given difficulties in traversing the blood brain barrier, it will be beneficial to achieve supratherapeutic levels of IDUA enzyme peripherally to facilitate transcytosis and ultimate delivery of a much lower but clinically relevant level of enzyme to difficult-to-access target organs^{36,37}. In addition, use of AAV, lipid nanoparticle, and other alternate delivery mechanisms should facilitate maximal delivery of base editing technology which must be balanced against the safety risks of the vector and the delivered transgene/effector¹⁷. Finally, our in vitro approach utilized mRNA which is compatible with clinically relevant lipid nanoparticle delivery in vivo, an approach that may portend reduced off-targeting due to the transience of mRNA transcript, rapid translation and bioavailability of active base editor protein, and lower risk of deleterious vector integration compared to traditional vehicles such as AAV^{17,54,59}

In summary, our experiments demonstrate precise adenine base-editor mediated correction of a common human mutation in a lysosomal storage disease with unmet therapeutic need.

### References for Example III

1. Ahmed, A. et al. Mucopolysaccharidosis (MPS) Physical Symptom Score: Development, Reliability, and Validity. in JIMD Reports, Volume 26 (eds. Morava, E. et al.) 61-68 (Springer, 2016). doi:10.1007/8904_2015_485.

2. Braunlin, E. A. et al. Cardiac disease in patients with mucopolysaccharidosis: presentation, diagnosis and management. J Inherit Metab Dis 34, 1183-1197 (2011).

3. Tomatsu, S. et al. Dermatan sulfate and heparan sulfate as a biomarker for mucopolysaccharidosis I. J Inherit Metab Dis 33, 141-150 (2010).

4. Pereira, V. G. et al. Evidence of lysosomal membrane permeabilization in mucopolysaccharidosis type I: Rupture of calcium and proton homeostasis. Journal of Cellular Physiology 223, 335-342 (2010).

5. Miebach, E. Enzyme replacement therapy in mucopolysaccharidosis type I. Acta Paediatrica 94, 58-60 (2005).

6. Guffon, N. et al. Long term disease burden post-transplantation: three decades of observations in 25 Hurler patients successfully treated with hematopoietic stem cell transplantation (HSCT). Orphanet Journal of Rare Diseases 16, NA-NA (2021).

7. Gentner, B. et al. Ex-Vivo Gene Therapy for Hurler Disease: Initial Results from a Phase I/II Clinical Study. in Clinical Trials Spotlight vol. 27 1-2 (Molecular Therapy, 2019).

8. Gene Therapy With Modified Autologous Hematopoietic Stem Cells for the Treatment of Patients With Mucopolysaccharidosis Type I, Hurler Variant — Full Text View —Clinical Trials.gov. https://clinicaltrials.gov/ct2/show/NCT03488394 (2020).

9. de Carvalho, T. G. et al. CRISPR-Cas9-mediated gene editing in human MPS I fibroblasts. Gene 678, 33-37 (2018).

10. Gomez-Ospina, N. et al. Human genome-edited hematopoietic stem cells phenotypically correct Mucopolysaccharidosis type I. Nat Commun 10, 4045 (2019).

11. Gaudelli, N. M. et al. Programmable base editing of A•T to G•C in genomic DNA without DNA cleavage. Nature 551, 464-471 (2017).

12. Gaudelli, N. M. et al. Directed evolution of adenine base editors with increased activity and therapeutic application. Nat Biotechnol 38, 892-900 (2020).

13. Koblan, L. W. et al. Improving cytidine and adenine base editors by expression optimization and ancestral reconstruction. Nature Biotechnology 36, 843-846 (2018).

14. Jin, S. et al. Cytosine, but not adenine, base editors induce genome-wide off-target mutations in rice. Science 364, 292-295 (2019).

15. Levy, J. M. et al. Cytosine and adenine base editing of the brain, liver, retina, heart and skeletal muscle of mice via adeno-associated viruses. Nature Biomedical Engineering 4, 97-110 (2020).

16. Koblan, L. W. et al. In vivo base editing rescues Hutchinson-Gilford progeria syndrome in mice. Nature 589, 608-614 (2021).

17. Riley, R. S. et al. Ionizable lipid nanoparticles for in utero mRNA delivery. Science Advances 7, eaba1028 (2021).

18. Zhang, X. et al. Functionalized lipid-like nanoparticles for in vivo mRNA delivery and base editing. Science Advances 6, eabc2315 (2020).

19. Musunuru, K. et al. In vivo CRISPR base editing of PCSK9 durably lowers cholesterol in primates. Nature 593, 429-434 (2021).

20. Song, C.-Q. et al. Adenine base editing in an adult mouse model of tyrosinaemia. Nature Biomedical Engineering 4, 125-130 (2020).

21. Bose, S. K. et al. In utero adenine base editing corrects multi-organ pathology in a lethal lysosomal storage disease. Nat Commun 12, 4291 (2021).

22. Homo sapiens alpha-L-iduronidase (IDUA), transcript variant 2, non-cod - Nucleotide -NCBI.

https://www.ncbi.nlm.nih.gov/nucleotide/NR_110313.1?report=genbank&log$=nuclalign&blast _rank=28&RID=9TO9FS8CO16.

23. Swaroop, M., Brooks, M. J., Gieser, L., Swaroop, A. & Zheng, W. Patient iPSC-derived neural stem cells exhibit phenotypes in concordance with the clinical severity of mucopolysaccharidosis I. Human Molecular Genetics 27, 3612-3626 (2018).

24. Hopwood, J. J. & Muller, V. Biochemical discrimination of hurler and scheie syndromes. Pathology 11, 327 (1979).

25. Bunge, S. et al. Genotype-phenotype correlations in mucopolysaccharidosis type I using enzyme kinetics, immunoquantification and in vitro turnover studies. Biochimica et Biophysica Acta (BBA) - Molecular Basis of Disease 1407, 249-256 (1998).

26. Pierzynowska, K., Gaffke, L., Podlacha, M., Brokowska, J. & Węgrzyn, G. Mucopolysaccharidosis and Autophagy: Controversies on the Contribution of the Process to the Pathogenesis and Possible Therapeutic Applications. Neuromol Med 22, 25-30 (2020).

27. Osteoimmunology in mucopolysaccharidoses type I, II, VI and VII. Immunological regulation of the osteoarticular system in the course of metabolic inflammation - ScienceDirect. https://www-sciencedirect-com.proxy.library.upenn.edu/science/article/pii/S1063458413009047.

28. Archer, L. D., Langford-Smith, K. J., Bigger, B. W. & Fildes, J. E. Mucopolysaccharide diseases: A complex interplay between neuroinflammation, microglial activation and adaptive immunity. J Inherit Metab Dis 37, 1-12 (2014).

29. Clarke, L. A., Winchester, B., Giugliani, R., Tylki-Szymańska, A. & Amartino, H. Biomarkers for the mucopolysaccharidoses: Discovery and clinical utility. Molecular Genetics and Metabolism 106, 395-402 (2012).

30. M, F. et al. Gene Therapy of Mucopolysaccharidosis Type I Mice: Repeated Administrations and Safety Assessment of pIDUA/Nanoemulsion Complexes. Curr Gene Ther (2021) doi:10.2174/1566523221666210126151420.

31. Pasqualim, G. et al. Effects of Enzyme Replacement Therapy Started Late in a Murine Model of Mucopolysaccharidosis Type I. PLOS ONE 10, e0117271 (2015).

32. Zampetti, A. et al. Vascular Endothelial Growth Factor (VEGF-a) in Fabry disease: Association with cutaneous and systemic manifestations with vascular involvement. Cytokine 61, 933-939 (2013).

33. Alterations in Oxidative Markers in the Cerebellum and Peripheral Organs in MPS I Mice.https://www.infona.pl/resource/bwmetal.element.springer-4d2cb603-81ac-3676-91cb-eabc8188ecb7.

34. Kakkis, E. et al. Intrathecal enzyme replacement therapy reduces lysosomal storage in the brain and meninges of the canine model of MPS I. Molecular Genetics and Metabolism 83, 163-174 (2004).

35. Genetic engineering of a lysosomal enzyme fusion protein for targeted delivery across the human blood-brain barrier — Boado — 2008 - Biotechnology and Bioengineering - Wiley Online Library.https://onlinelibrary-wiley-com.proxy.library.upenn.edu/doi/abs/10.1002/bit.21602?casa_token=U4N5_pjCMvAAAAAA:8 NLV9QXvjn7REySX6-GPgoIDxcgQfDpczmT68gsnQ301um6M42ZT5PQuQQtWraxk0B5ksqxznKFaFo.

36. Ou, L. et al. ZFN-Mediated In Vivo Genome Editing Corrects Murine Hurler Syndrome. Molecular Therapy 27, 178-187 (2019).

37. Ou, L. et al. A Highly Efficacious PS Gene Editing System Corrects Metabolic and Neurological Complications of Mucopolysaccharidosis Type I. Molecular Therapy 28, 1442-1454 (2020).

38. Hartung, S. D., Reddy, R. G., Whitley, C. B. & Mcivor, R. S. Enzymatic Correction and Cross-Correction of Mucopolysaccharidosis Type I Fibroblasts by Adeno-Associated Virus-Mediated Transduction of the alpha-L-Iduronidase Gene. Human Gene Therapy 10, 2163-2172 (1999).

39. Identification and Validation of Mannose 6-Phosphate Glycoproteins in Human Plasma Reveal a Wide Range of Lysosomal and Non-lysosomal Proteins | Molecular & Cellular Proteomics. https://www.mcponline.org/content/5/10/1942.short.

40. Kim, D. & Kim, J.-S. DIG-seq: a genome-wide CRISPR off-target profiling method using chromatin DNA. Genome Res. 28, 1894-1900 (2018).

41. Kim, D., Kim, S., Kim, S., Park, J. & Kim, J.-S. Genome-wide target specificities of CRISPR-Cas9 nucleases revealed by multiplex Digenome-seq. Genome Res 26, 406-415 (2016).

42. Ameur, A. Amplification-free long read sequencing reveals unforeseen CRISPR-Cas9 off-target activity. 20.

43. Dobbs, F. M. et al. Precision digital mapping of endogenous and induced genomic DNA breaks by INDUCE-seq. bioRxiv 2020.08.25.266239 (2020) doi:10.1101/2020.08.25.266239.

44. Petri, K. et al. Global-scale CRISPR gene editor specificity profiling by ONE-seq identifies population-specific, variant off-target effects.

http://biorxiv.org/lookup/doi/10.1101/2021.04.05.438458 (2021) doi:10.1101/2021.04.05.438458.

45. Ishizuka, J. J. et al. Loss of ADAR1 in tumours overcomes resistance to immune checkpoint blockade. Nature 565, 43-48 (2019).

46. Eisenberg, E. & Levanon, E. Y. A-to-I RNA editing - immune protector and transcriptome diversifier. Nature Reviews Genetics 19, 473-490 (2018).

47. Rees, H. A., Wilson, C., Doman, J. L. & Liu, D. R. Analysis and minimization of cellular RNA editing by DNA adenine base editors. Science Advances 5, eaax5717 (2019).

48. Ensembl Variant Effect Predictor (VEP). https://uswest.ensembl.org/info/docs/tools/vep/index.html.

49. Sim, N.-L. et al. SIFT web server: predicting effects of amino acid substitutions on proteins. Nucleic Acids Research 40, W452-W457 (2012).

50. Rentzsch, P., Witten, D., Cooper, G. M., Shendure, J. & Kircher, M. CADD: predicting the deleteriousness of variants throughout the human genome. Nucleic Acids Research 47, D886-D894 (2019).

51. Rossidis, A. C. et al. In utero CRISPR-mediated therapeutic editing of metabolic genes. Nature Medicine 24, 1513-1518 (2018).

52. Bunge, S. et al. Mucopolysaccharidosis type I: identification of 8 novel mutations and determination of the frequency of the two common α-L-iduronidase mutations (W402X and Q70X) among European patients. Hum Mol Genet 3, 861-866 (1994).

53. Oussoren, E. et al. Residual α-l-iduronidase activity in fibroblasts of mild to severe Mucopolysaccharidosis type I patients. Molecular Genetics and Metabolism 109, 377-381 (2013).

54. Miller, J. B. et al. Non-Viral CRISPR/Cas Gene Editing In Vitro and In Vivo Enabled by Synthetic Nanoparticle Co-Delivery of Cas9 mRNA and sgRNA. Angewandte Chemie International Edition 56, 1059-1063 (2017).

55. Chung, C.-H. et al. Computational Analysis Concerning the Impact of DNA Accessibility on CRISPR-Cas9 Cleavage Efficiency. Molecular Therapy 28, 19-28 (2020).

56. Jensen, K. T. et al. Chromatin accessibility and guide sequence secondary structure affect CRISPR-Cas9 gene editing efficiency. FEBS Letters 591, 1892-1901 (2017).

57. Chari, R., Mali, P., Moosburner, M. & Church, G. M. Unraveling CRISPR-Cas9 genome engineering parameters via a library-on-library approach. Nature Methods 12, 823-826 (2015).

58. Uusi-Mäkelä, M. I. E. et al. Chromatin accessibility is associated with CRISPR-Cas9 efficiency in the zebrafish (Danio rerio). PLOS ONE 13, e0196238 (2018).

59. Yin, H. et al. Therapeutic genome editing by combined viral and non-viral delivery of CRISPR system components in vivo. Nature Biotechnology 34, 328-333 (2016).

60. Kluesner, M. G. et al. EditR: A Method to Quantify Base Editing from Sanger Sequencing. The CRISPR Journal 1, 239-250 (2018).

61. Clement, K. et al. CRISPResso2 provides accurate and rapid genome editing sequence analysis. Nature Biotechnology 37, 224-226 (2019).

62. Park, J. et al. Digenome-seq web tool for profiling CRISPR specificity. Nature Methods 14, 548-549 (2017).

63. Trimmomatic: a flexible trimmer for Illumina sequence data | Bioinformatics | Oxford Academic. https://academic.oup.com/bioinformatics/article/30/15/2114/2390096.

64. STAR: ultrafast universal RNA-seq aligner | Bioinformatics | Oxford Academic. https://academic.oup.com/bioinformatics/article/29/1/15/272537.

65. Moderated estimation of fold change and dispersion for RNA-seq data with DESeq2 | Genome Biology | Full Text. https://genomebiology.biomedcentral.com/articles/10.1186/s13059-014-0550-8.

66. Perampalam, P. & Dick, F. A. BEAVR: a browser-based tool for the exploration and visualization of RNA-seq data. BMC Bioinformatics 21, (2020).

67. Picardi, E. & Pesole, G. REDItools: high-throughput RNA editing detection made easy. Bioinformatics 29, 1813-1814 (2013).

68. Danecek, P. et al. The variant call format and VCFtools. Bioinformatics 27, 2156-2158 (2011).

### Example IV

### Clinical Application of in Vivo Gene Editing for MPS-IH

Clinical application of in vivo gene editing for MPS-IH or other lysosomal storage diseases can be achieved using several different approaches. Using in vivo base editing for MPS-IH as an example, adenine base editing to correct the common G-to-A (W402X) mutation in the human IDUA gene could be applied in the prenatal or postnatal MPS-IH patient. In all patient populations, molecular diagnosis would confirm the presence of the mutation. This would be performed by genetic testing methods frequently employed by clinicians in this art area, including, but not limited to, a skin biopsy specimen from a postnatal patient. Alternatively, for fetal therapy, multiple pathways for prenatal diagnosis exist. In families with no known history of MPS-IH, commercial carrier screens can identify pregnancies at risk for MPS-IH (e.g., Progenity Preparent Carrier Test and Integrated Genetics 500 Plus Panel). Alternatively, in families at high-risk of MPS-IH or with known affected offspring, minimally invasive ultrasound guided amniocentesis and chorionic villus sampling (CVS) have been used to prenatally diagnose MPS-IH affected fetuses with high specificity and sensitivity based on elevated GAGs and reduced IDUA enzyme activity.^{1,2} Moreover, amniocentesis in five pregnant women led to prenatal diagnosis in six fetuses on the basis of direct sequencing of the IDUA gene.³ Thus, any of these approaches are suitable for MPS-IH diagnosis in prenatal and postnatal patients and the genetic mutations, including the common W402X mutation, responsible for the disease can be identified.

After confirming the diagnosis of MPS-IH and the genetic mutation associated with the disease, the gRNA targeting the human W402X IDUA mutation will be screened in vitro to confirm the lack of significant off-target activity including, individual single nucleotide polymorphisms (SNPs) that may be unique to the patient. Techniques to perform this screening include, but are not limited to, ONEseq described above.⁴

Following clinical and genetic diagnosis of MPS-IH, administration of the gene therapy would then be performed using techniques already established for systemic (intravenous) and/or central nervous system (CNS; intra-thecal, intra-cisterna magna, intra-ventricular) delivery in the prenatal and postnatal patient using either an AAV, LNP, or other suitable delivery vehicle. For example, a similar approach and dose that has been used to intravenously delivery AAV9 carrying the survival motor neuron 1 (SMN) complementary DNA to treat spinal muscular atrophy type 1 (SMA1) patients would be used.⁵ In this case, patients would be given prednisolone (1 mg/kg/day) for approximately 30 days beginning 1 day prior to vector administration. The total dose of AAV administered intravenously would ~ 1x10¹⁴ vg / kg although lesser doses may be required based on toxicity. The vector will be delivered in normal saline over a 60 minute infusion time. As an alternative approach, LNP delivery of mRNA encoding the base editing enzyme (ABE8.8-m for example) and the IDUA targeting gRNA can be delivered intravenously to correct the disease-causing mutation in MPS-IH in a technical approach similar to that used to treat Transthyretin Amyloidosis.⁶ In this case, patients were intravenously administered an LNP containing a total RNA dose of 0.1-0.3 mg / kg. Of note, dose ranges may be higher or lower for MPS-IH and other lysosomakl storage diseases. In addition to systemic delivery by the above methods, CNS targeted delivery of the base editing therapy for MPS-IH and other lysosomal storage and monogenic diseases would be performed independently or together with systemic delivery. The technical approach for CNS targeted delivery would be similar to that employed for clinical trials in SMA as well as a number of preclinical studies for lysosomal storage diseases and includes intrathecal and intracisterna magna injection of the AAV vector, LNP or other suitable delivery vehicle carrying the therapeutic editing transgene or mRNA.^{7,8}

Finally, for treatment of the fetal patient, multiple examples exist in clinical as well as preclinical studies for intravascular and CNS targeted delivery of a therapeutic. Intravascular delivery of gene editing technology to the fetus would be accomplished via the umbilical vein injection. This would be a minimally invasive procedure performed under ultrasound guidance and local anesthesia without a maternal laparotomy. The procedure for an umbilical vein injection would be the same as that which occurs for umbilical vein transfusions which are performed routinely, often multiple times during pregnancy, for select fetuses diagnosed with anemia.⁹ This procedure would be performed at 18 weeks gestation and older. Alternatively, we have performed fetal intracardiac hematopoietic stem cell transplantations at 16 weeks gestation and thus this approach lends itself to earlier gestation delivery of the gene therapy. Fetal CNS delivery of gene therapy viral vectors has also been performed in mouse models and preclinical nonhuman primate models and the same approach would be used alone or in combination with systemic delivery of viral vectors, LNPs, or other suitable delivery vehicle carrying transgenes or mRNA for base editing to treat MPS-IH and other lysosomal storage diseases.¹⁰

### References

1. Akella, R. R. D. & Kadali, S. Amniotic fluid glycosaminoglycans in the prenatal diagnosis of mucopolysaccharidoses-A useful biomarker. Clin. Chim. Acta 460, 63-66 (2016).

2. Nasr, A. A. & Fateen E. Prenatal diagnosis of mucopolysaccharidoses (MPS): the first Egyptian experience. Bratisl Lek Listy 105, 310-314 (2004).

3. Wang, X. et al. Mucopolysaccharidosis I mutations in Chinese patients: identification of 27 novel mutations and 6 cases involving prenatal diagnosis. Clin. Genet. 81, 443-452 (2012).

4. Petri, K. et al. Global-scale CRISPR gene editor specificity profiling by ONE-seq identifies population-specific, variant off-target effects. Preprint at https://doi.org/10.1101/2021.04.05. 438458 (2021).

5. Mendell JR et al. Single-Dose Gene Replacement Therapy for Spinal Muscular Atrophy. NEJM. 377, 1713-1722 (2017).

6. Gillmore et al. CRISPR-Cas9 In Vivo Gene Editing for Transthyretin Amyloidosis. NEJM, Jun 26. doi: 10.1056/NEJMoa2107454. (2021).

7. Darras et al. An Integrated Safety Analysis of Infants and Children with Symptomatic Spinal Muscular Atrophy (SMA) Treated with Nusinersen in Seven Clinical Trials. CNS Drugs. 33, 919-932 (2019).

8. Bradbury AM et al. Krabbe disease successfully treated via monotherapy of intrathecal gene therapy. Journal of Clinical Investigation. 130, 5906-4920 (2020).

9. Zwiers, C. et al. Complications of intrauterine intravascular blood transfusion: lessons learned after 1678 procedures. Ultrasound Obstet. Gynecol. 50, 180-186 (2017).

10. Massaro G et al. Fetal gene therapy for neurodegenerative disease of infants. Nature Medicine. 24, 1317-1323 (2018).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An adenine base editor (ABE) complex for programming conversion of adenine to guanine in a patient in need thereof, said patient have a target DNA molecule harboring a mutation associated with a lysosomal storage disease comprising, a modified TadA enzyme, a catalytically impaired Cas 9 protein and a single guide RNA (sgRNA) which directs said ABE complex to said mutated target DNA molecule, which upon contact converts adenine in said mutation to inosine, thereby catalyzing an A-T to G-C transition following DNA replication.

2. The ABE complex of claim 1 wherein said ABE is selected from ABEmax, ABE6.3, ABE6.4, ABE7.8, ABE7.9, ABE7.10, ABE7.10-m, ABE7.10-d, ABE8.8-m, ABE8.8-d, ABE8.13-m, ABE8.13-d, ABE8.17-m, ABE8.17-d, ABE8.20-m and ABE8.20-d.

3. The ABE complex of claim 1 for the treatment of Hurler syndrome, wherein said catalytically impaired Cas9 protein is selected from NRRH, NRTH, NRCH, xCas9, SpCas9-NG, SpCas9, SpG, SpRY, SauriCas9, SaCas9, Nme2Cas9, VRER-SpCas9, and VQR-SpCas9.

4. The ABE complex of claim 1,
wherein the target base position is italicized and the complex and protospacer and PAM sequences are selected from
i) spCas9.ABEmax and GCTCTAGGCCGAAGTGTCGC AGG;
ii) spCas9.ABEmax and TAGGCCGAAGTGTCGCAGGC and CGG;
iii) Nme2Cas9.ABEmax and GAGCAGCTCTAGGCCGAAGTGTCG and CAGGCC;
iv) Nme2Cas9.ABEmax and CTCTAGGCCGAAGTGTCGCAGGCC and GGGACC;
v) SpG.ABEmax CTCTAGGCCGAAGTGTCGCA and GGCC;
vi) SpRY.ABEmax CTCTAGGCCGAAGTGTCGCA and GGCC;
vii) SauriCas9.ABEmax AGCTCTAGGCCGAACTCTCG and CAGG; and
viii) NRCH.ABEmax GCAGCTCTAGGCCGAAGTGT and CGCA.

5. The ABE complex of claim 1, wherein said lysosomal storage disease is Hurler’s syndrome, said target DNA sequence comprises a W402X mutation present in an Idua gene and said G→A conversion restores Idua activity, said conversion occurring in the absence of a double strand break.

6. The ABE complex of claim 5, wherein said ABE complex is ABE7.10 and said guide strand comprises a protospacer and PAM sequence of 5′GCTCTAGGCCGAAGTGTCGCAGG3′, said restoration of Idua activity ameliorates symptoms of Hurler’s disease.

7. The ABE complex of claim 1, wherein said complex is delivered to said patient in a vector.

8. The ABE complex of claim 7, wherein said vector is selected from an adenoviral vector, at least one adeno-associated viral vector (AAV), a lentiviral vector, a retroviral vector and a plasmid.

9. The ABE complex of claim 7, wherein a first and second AAV9 vector are administered.

10. The ABE complex of claim 1, which is delivered to said patient in as a nucleoprotein complex or mRNA in a lipid based nanoparticle.

11. A method for genome editing of a mutated gene sequence associated with a lysosomal storage disease in a patient in need thereof, the method comprising:
administering to the patient an ABE complex as claimed in claim 1 which introduces a modified codon into said gene sequence and corrects said mutation, wherein the base editing does not induce double strand breaks in the target nucleic acid and said correction of said mutation ameliorates symptoms of said lysosomal storage disease.

12. The method of claim 11, wherein said patient is in utero or selected from a neonate, child or adult, and said ABE corrects the human INDUA G → A, W402X mutation present in Hurler syndrome patients.

13. The method of claim 12, wherein the base editing occurs prior to Hurler syndrome onset.

14. The method of claim 1, wherein the base editing occurs in the fetus, wherein the fetus is inside a uterus of a body of a living carrier.

15. The method of claim 11, wherein the base editing decreases a risk of developing a disease.

16. The method of claim 12, wherein said ABE complex is ABE7.10 and said guide strand comprises a protospacer and PAM sequence of 5′GCTCTAGGCCGAAGTGTCGCAGG3′, said restoration of Idua activity ameliorates symptoms of Hurler’s disease.

17. The method of claim 16, wherein said ABE complex is delivered as a nucleoprotein or mRNA.

18. The method of claim 11, wherein said complex is delivered in at least one AAV vector having a serotype selected from AAV1, AAV2, AAV4, AAV5, AAV6, AAV7, AAV8, and AAV9.

19. The method of claim 16, wherein said complex is delivered in first and second AAV9 vectors wherein amplified N- and C-termini of the ABEmax are ligated at SpCas9 Glu573 and Cys574 to codon optimized N- and C-termini of the Npu intein, respectively, codons for Leu564 and Lys565 in said Npu intein being altered to an AflII site (CTT|AAG) to form plasmids, said plasmids comprising a CBh promoter and WPRE3-bGH polyadenylation signal sequences and being inserted between AAV ITRs in said first and second vectors.

20. A cytosine base editor (CBE) complex for programming conversion of cytosine into a thymine in a patient in need thereof said patient have a target DNA molecule harboring a mutation associated with a lysosomal storage disease comprising, a cytosine deaminase domain, a catalytically impaired Cas 9 protein and a single guide RNA (sgRNA) which directs said CBE complex to said mutated target DNA molecule, which upon contact converts cytosine in said mutation to inosine, thereby catalyzing an C→T transition following DNA replication.

21. The complex of claim 20, wherein said catalytically impaired Cas9 protein is selected from NRRH, NRTH, NRCH, xCas9, SpCas9-NG, SpCas9, SpG, SpRY, SauriCas9, SaCas9, Nme2Cas9, VRER-SpCas9, and VQR-SpCas9.

22. The complex of claim 20 wherein said CBE is selected from APOBEC1, E63A, CDA, AID, A3A, A3B, A3G, YE1, YE2, YEE, EE, R33A, eA3A, FERNY, BE3, BE4, and BE4max.

23. A method for treating a lysosomal storage disease in a fetal, neonate, child or adult subject, the method comprising:
(a) identifying in vitro a target codon for base editing;
(b) providing an ABE or CBE complex,
(c) administering said complex to the subject; thereby introducing a modified codon in a mutated gene sequence and ameliorating symptoms of said lysosomal storage disease in said subject.

24. The method of claim 23, wherein the subject is a fetus and base editing occurs prior to disease onset, wherein the disease is a phenotype resulting from the mutation in the therapeutic gene.

25. The method of claim 20, wherein the base editing decreases a risk of developing a disease.

26. The method of claim 23, wherein the therapeutic gene is base edited in an embryo, wherein the base editing is performed prior to implantation of the embryo into a uterus of a carrier, wherein the carrier is a mammal.

27. The method of claim 25, wherein the mammal is a human.

28. The method of claim 25, wherein the mammal is an animal.

29. The method of claim 25, wherein the embryo is base edited in vivo in the uterus after in vitro fertilization.

30. The method of claim 25, wherein the embryo is base edited in vitro after in vitro fertilization.


SEQUENCE LISTING (97 sequences)

SEQ ID NO: 1 - DNA, 23 bp, Artificial Sequence
  spCas9.ABEmax
gctctaggccgaagtgtcgcagg

SEQ ID NO: 2 - DNA, 23 bp, Artificial Sequence
  spCas9.ABEmax
taggccgaagtgtcgcaggccgg

SEQ ID NO: 3 - DNA, 30 bp, Artificial Sequence
  Nme2Cas9.ABEmax
gagcagctctaggccgaagtgtcgcaggcc

SEQ ID NO: 4 - DNA, 30 bp, Artificial Sequence
  Nme2Cas9.ABEmax
ctctaggccgaagtgtcgcaggccgggacc

SEQ ID NO: 5 - DNA, 24 bp, Artificial Sequence
  SpG.ABEmax
ctctaggccgaagtgtcgcaggcc

SEQ ID NO: 6 - DNA, 24 bp, Artificial Sequence
  SpRY.ABEmax
ctctaggccgaagtgtcgcaggcc

SEQ ID NO: 7 - DNA, 24 bp, Artificial Sequence
  SauriCas9.ABEmax
agctctaggccgaactctcgcagg

SEQ ID NO: 8 - DNA, 24 bp, Artificial Sequence
  NRCH.ABEmax
gcagctctaggccgaagtgtcgca

SEQ ID NO: 9 - DNA, 22 bp, Artificial Sequence
  E15.5 AAV.ABE.Idua-W392X
ctctaggcagaggtctcaaagg

SEQ ID NO: 10 - DNA, 23 bp, Artificial Sequence
  Exon:idua
actctaggcagaggtctcaaagg

SEQ ID NO: 11 - DNA, 23 bp, Artificial Sequence
  Intron: 1700010l14Rik
gttctagactgaggtctcaaggg

SEQ ID NO: 12 - DNA, 23 bp, Artificial Sequence
  Intron:Wnt11
actccaagctggggtctcaacgg

SEQ ID NO: 13 - DNA, 23 bp, Artificial Sequence
  Intergenic:Papl-Fbxo27
actctaggctagagtctcaaagg

SEQ ID NO: 14 - DNA, 23 bp, Artificial Sequence
  Intergenic:Fgf9-Gm25614
acttttgacagaggtatcaaggg

SEQ ID NO: 15 - DNA, 23 bp, Artificial Sequence
  Intergenic:Gm12106-Stc2
attccagccagaggtatcaaagg

SEQ ID NO: 16 - DNA, 23 bp, Artificial Sequence
  Intergenic:Gm26190-B3gat1
agttcagacagaggtctcaaagg

SEQ ID NO: 17 - DNA, 23 bp, Artificial Sequence
  Intergenic:Ccdc85c-Hhipl1
gctccaggcagaggtcccagggg

SEQ ID NO: 18 - DNA, 23 bp, Artificial Sequence
  Intron:Osbpl1a
gaactaagcagaggtctcaaagg

SEQ ID NO: 19 - DNA, 23 bp, Artificial Sequence
  Intron:Blnk
gctctgagcagaggtcccaacgg

SEQ ID NO: 20 - DNA, 23 bp, Artificial Sequence
  IntronLRhoj
actctacacagaggtaccaatgg

SEQ ID NO: 21 - DNA, 10 bp, Artificial Sequence
  ABE7.10 mRNA
ggcctagagc

SEQ ID NO: 22 - DNA, 23 bp, Artificial Sequence
  loxP targeting protospacer and PAM
attatacgaagttatattaaggg

SEQ ID NO: 23 - DNA, 20 bp, Artificial Sequence
  loxP targeting protospacer
attatacgaagttatattaa

SEQ ID NO: 24 - DNA, 20 bp, Artificial Sequence
  Mouse IDUA protospacer
actctaggcagaggtctcaa

SEQ ID NO: 25 - DNA, 20 bp, Artificial Sequence
  Primer Idua-F
tgctaggtatgagagagcca

SEQ ID NO: 26 - DNA, 21 bp, Artificial Sequence
  Primer Idua-R
agtgtagatgaggactgtggt

SEQ ID NO: 27 - DNA, 20 bp, Artificial Sequence
  Primer Intron:1700010I14Rik F
gggattgctctgctctgtct

SEQ ID NO: 28 - DNA, 20 bp, Artificial Sequence
  Primer Intron:1700010I14Rik R
tgtgtaagagtgggccatgt

SEQ ID NO: 29 - DNA, 20 bp, Artificial Sequence
  Primer Intron:Wnt11 F
caggcttgaacacacacaca

SEQ ID NO: 30 - DNA, 20 bp, Artificial Sequence
  Primer Intron:Wnt11 R
aaaatcccgttgagacccca

SEQ ID NO: 31 - DNA, 21 bp, Artificial Sequence
  Primer Intergenic:Papl-Fbxo27 F
caacatttggaagtctgaggc

SEQ ID NO: 32 - DNA, 19 bp, Artificial Sequence
  Primer Intergenic:Papl-Fbxo27 R
tgctggggttacaagggtg

SEQ ID NO: 33 - DNA, 21 bp, Artificial Sequence
  Primer Intergenic:Fgf9-Gm25614 F
actgcaggaatggaaaactcc

SEQ ID NO: 34 - DNA, 20 bp, Artificial Sequence
  Primer Intergenic:Fgf9-Gm25614 R
ctctagagaccctgtgctgg

SEQ ID NO: 35 - DNA, 20 bp, Artificial Sequence
  Primer Intergenic:Gm12106-Stc2 F
aggccttcgatcagacatca

SEQ ID NO: 36 - DNA, 20 bp, Artificial Sequence
  Primer Intergenic:Gm12106-Stc2 R
caacaacatggctgctcagg

SEQ ID NO: 37 - DNA, 20 bp, Artificial Sequence
  Primer Intergenic:Gm26190-B3gat1 F
ccttcactctcttgggcctt

SEQ ID NO: 38 - DNA, 20 bp, Artificial Sequence
  Primer Intergenic:Gm26190-B3gat1 R
cagtgtcagcaaagggaagc

SEQ ID NO: 39 - DNA, 20 bp, Artificial Sequence
  Primer Intergenic:Ccdc85c-Hhipl1 F
acaaggaggggtgtgtgtac

SEQ ID NO: 40 - DNA, 19 bp, Artificial Sequence
  Primer Intergenic:Ccdc85c-Hhipl1 R
ctgctgagaggtcctggag

SEQ ID NO: 41 - DNA, 21 bp, Artificial Sequence
  Primer Intron:Osbpl1a F
gcccacttaataaccctgtgt

SEQ ID NO: 42 - DNA, 20 bp, Artificial Sequence
  Primer Intron:Osbpl1a R
gcaggaggggtcattgatct

SEQ ID NO: 43 - DNA, 20 bp, Artificial Sequence
  Primer Intron:Blnk F
acagcactgagaagggacaa

SEQ ID NO: 44 - DNA, 20 bp, Artificial Sequence
  Primer Intron:Blnk R
cgggagggatcgtaaagtga

SEQ ID NO: 45 - DNA, 20 bp, Artificial Sequence
  Primer Intron:Rhoj F
ttggctagtctccgtgtgaa

SEQ ID NO: 46 - DNA, 20 bp, Artificial Sequence
  Primer Intron:Rhoj R
ggggtctagaggtctttggg

SEQ ID NO: 47 - DNA, 17 bp, Artificial Sequence
  Primer CAIDUA_F
ctggcgctgctggccgc

SEQ ID NO: 48 - DNA, 20 bp, Artificial Sequence
  Primer CAIDUA_R
ctggacggtcccgggccctg

SEQ ID NO: 49 - DNA, 20 bp, Artificial Sequence
  dMPS1.1
ttctgatgagggctccgcgg

SEQ ID NO: 50 - DNA, 20 bp, Artificial Sequence
  dMPS1.2
gcttctgatgagggctccgc

SEQ ID NO: 51 - DNA, 20 bp, Artificial Sequence
  dMPS1.3
ggcttctgatgagggctccg

SEQ ID NO: 52 - DNA, 20 bp, Artificial Sequence
  dMPS1.4
cttctgatgagggctccgcg

SEQ ID NO: 53 - DNA, 20 bp, Artificial Sequence
  dMPS4
tctgatgagggctccgcggg

SEQ ID NO: 54 - DNA, 20 bp, Artificial Sequence
  human IDUA protospacer
gctctaggccgaagtgtcgc

SEQ ID NO: 55 - DNA, 22 bp, Artificial Sequence
  Hu.IDU-F
caatgccttcctgagctaccac

SEQ ID NO: 56 - DNA, 20 bp, Artificial Sequence
  Hu.IDUA-R
aggtagcgcgtgacgtagac

SEQ ID NO: 57 - DNA, 23 bp, Artificial Sequence
  Chr10 Exon: MIR3663HG
gctctgggctggggtgtcgctgg

SEQ ID NO: 58 - DNA, 23 bp, Artificial Sequence
  Chr8 Intergenic: MIR3686-RP11-419K12.1
gctctaggctgaagtgcttctgg

SEQ ID NO: 59 - DNA, 23 bp, Artificial Sequence
  Chr19 Intergenic: RAB11B-AS1/RAB11B-RAB11B
ccactaggccaaagtgtagctgg

SEQ ID NO: 60 - DNA, 23 bp, Artificial Sequence
  Chr3 Intergenic: RBM15B/VPRBP-VPRBP
gctccaggaggaagtgtcacagg

SEQ ID NO: 61 - DNA, 23 bp, Artificial Sequence
  Chr8 Intron: DLC1
gttctaggtggaagtgttgctgg

SEQ ID NO: 62 - DNA, 23 bp, Artificial Sequence
  Chr17 Intergenic: ASIC2-AA06
ggtctaggccaagctgtcgctgg

SEQ ID NO: 63 - DNA, 23 bp, Artificial Sequence
  ChrX Intron: LINC01456
gctctaggcagaagagttgacgg

SEQ ID NO: 64 - DNA, 23 bp, Artificial Sequence
  Chr22 Intergenic: IGLV10-54-VPREB1
gatcaaggctgaagtgtccctgg

SEQ ID NO: 65 - DNA, 23 bp, Artificial Sequence
  Chr4 Intergenic: RP11-1398P2.1-FAM53A
gctccaggcccaaatgtccctgg

SEQ ID NO: 66 - DNA, 23 bp, Artificial Sequence
  Chr13 Intergenic: ENSG279924
gctctaggccgaagtgtcgcagg

SEQ ID NO: 67 - DNA, 23 bp, Artificial Sequence
  Chr1 Intergenic: CCDC17-GPBP1L1
gctctaggccgaagaggcgggag

SEQ ID NO: 68 - DNA, 23 bp, Artificial Sequence
  Chr14 Intron: FUT8
actataggctgaagtgtcctggg

SEQ ID NO: 69 - DNA, 23 bp, Artificial Sequence
  Chr22 Exon: GRAMD4
gctctagaacgaagttcacaggt

SEQ ID NO: 70 - DNA, 23 bp, Artificial Sequence
  Chr3 Intron: KALRN
tcactaggccaaagtgtatctgg

SEQ ID NO: 71 - DNA, 23 bp, Artificial Sequence
  Chr18 Intron: L3MBTL4
tctctaggtcagaagtgtcactg

SEQ ID NO: 72 - DNA, 23 bp, Artificial Sequence
  Chr12 Intergenic: LINC02368-LOC107987176
gctttaggcagaagtgtcaagga

SEQ ID NO: 73 - DNA, 23 bp, Artificial Sequence
  Chr5 Intergenic: PPIGP1-SGCD
gctctaaggccaaagtgtgcttg

SEQ ID NO: 74 - DNA, 23 bp, Artificial Sequence
  Chr14 Intergenic: BEGAIN-LINC00523
tcactaggcaaagtgtcacaggc

SEQ ID NO: 75 - DNA, 23 bp, Artificial Sequence
  Chr7 Intron: KLHDC10
gctccaggcaaagtgtctaaggg

SEQ ID NO: 76 - DNA, 23 bp, Artificial Sequence
  Chr4 Intron: MAN2B2
gctctatgccgaagtgttcggaa

SEQ ID NO: 77 - DNA, 23 bp, Artificial Sequence
  Chr13 Intron: FOXO1
gctcaggccggagtgtcgtggca

SEQ ID NO: 78 - DNA, 23 bp, Artificial Sequence
  Chr19 Intergenic: LINC01801
agactaggctgaagtgtcccagg

SEQ ID NO: 79 - DNA, 23 bp, Artificial Sequence
  Chr19 Intergenic: DAND5-NFIX
catctaggggaagtgtcactggg

SEQ ID NO: 80 - DNA, 23 bp, Artificial Sequence
  Chr15 Intron: ARNT2
actctaggtcaaagtgtcagctg

SEQ ID NO: 81 - DNA, 23 bp, Artificial Sequence
  Chr16 Intron: GSE1
actctaggcccaatgtcccaggg

SEQ ID NO: 82 - DNA, 23 bp, Artificial Sequence
  Chr1 Unchar: LOC105378839
tctctaggacaaagttgtctcag

SEQ ID NO: 83 - DNA, 23 bp, Artificial Sequence
  Chr5 Intergenic: LOC105377702-LINC01938
gctctattagaagtgtagcagga

SEQ ID NO: 84 - DNA, 23 bp, Artificial Sequence
  Chr3 Intron: SOX2-OT
tctctaggccgaagtcttaggga

SEQ ID NO: 85 - DNA, 23 bp, Artificial Sequence
  Chr20 Unchar: LOC105372578
tctctaggctgaagtgccctggg

SEQ ID NO: 86 - DNA, 23 bp, Artificial Sequence
  Chr13 Intergenic: MIR5007-HNF4GP1
tctctaaggaagtgtcacaggag

SEQ ID NO: 87 - DNA, 23 bp, Artificial Sequence
  Chr1 Intron: WDR47
gctctagaccaaagtggtctagg

SEQ ID NO: 88 - DNA, 23 bp, Artificial Sequence
  Chr10 Intron: PARD3
gctctagggccaaaggtcacagg

SEQ ID NO: 89 - DNA, 23 bp, Artificial Sequence
  Chr20 Intergenic: SLC2A10-RN7SKP33
gctctaagcagtagtgtgctggt

SEQ ID NO: 90 - DNA, 23 bp, Artificial Sequence
  Chr4 Intergenic: LOC105377476-MIR584G
tctctaggcagaagtgatgctgg

SEQ ID NO: 91 - DNA, 23 bp, Artificial Sequence
  Chr18 Intron: ZBTB7C
gctctaggcaggagggtccctcc

SEQ ID NO: 92 - DNA, 23 bp, Artificial Sequence
  Chr1 Intron: TRABD2B
gctctaaggaagtgtggcaggtg

SEQ ID NO: 93 - DNA, 23 bp, Artificial Sequence
  Chr10 Exon: CHT25H
actccatgtcgaagagtagcagg

SEQ ID NO: 94 - DNA, 23 bp, Artificial Sequence
  Chr17 Intron: Septin9
gctctaagatggagtgtcccagg

SEQ ID NO: 95 - DNA, 23 bp, Artificial Sequence
  Chr2 Intergenic: HSPE1P9-ARL4C
actctaggaagaagtgactcagg

SEQ ID NO: 96 - DNA, 23 bp, Artificial Sequence
  Chr3 Intron: NEC11
tctctaggcctaagtgtggaaag

SEQ ID NO: 97 - DNA, 23 bp, Artificial Sequence
  ABE7.10mRNA
gcgacacttcggcctagagctcc